(19) United States
(12) Patent Application Publication
(10) Pub. No.: US 2025/0122491 A1
(43) Pub. Date: Apr. 17, 2025

(54) OPTIMIZED PROTEIN FUSIONS AND LINKERS

(71) Applicant: Integrated DNA Technologies, Inc., Coralville, IA (US)

(72) Inventors: Michael Allen Collingwood, North Liberty, IA (US); Steve Ehren Glenn, Iowa City, IA (US); Christopher Anthony Vakulskas, North Liberty, IA (US)

(73) Assignee: Integrated DNA Technologies, Inc., Coralville, IA (US)

(21) Appl. No.: 18/908,672

(22) Filed: Oct. 7, 2024

Related U.S. Application Data
(63) Continuation of application No. 17/235,921, filed on Apr. 20, 2021, now Pat. No. 12,152,258.
(60) Provisional application No. 63/012,658, filed on Apr. 20, 2020.

(51) Int. Cl. C12N 9/22; C07K 14/47

(52) U.S. Cl. CPC C12N 9/22; C07K 14/47; C07K 2317/24

(57) ABSTRACT

This invention pertains to optimized protein fusion linkers for creating multi-functional chimeric proteins and methods of using the same. Additionally, the invention pertains to chimeric proteins for use in guided endonuclease systems and methods of using the same.

### CROSS-REFERENCE TO RELATED APPLICATIONS

This application is a continuation of U.S. Non-provisional patent application Ser. No. 17/235,921, filed Apr. 20, 2021 and titled “OPTIMIZED PROTEIN FUSIONS AND LINKERS,” which claims benefit of priority under 35 U.S.C. 119 to U.S. Provisional Patent Application Ser. No. 63/012,658, filed Apr. 20, 2020 and titled “OPTIMIZED PROTEIN FUSIONS AND LINKERS,” the contents of both applications which are herein incorporated by reference in their entirety.

### FIELD OF THE INVENTION

This invention pertains to the chimeric proteins and methods for their use in living cells.

### SEQUENCE LISTING

The instant application contains a Sequence Listing that has been submitted in XML format via Patent Center and is hereby incorporated by reference in its entirety. The XML copy, created on Oct. 7, 2024, is named IDT01-019-US-CON.XXML, and is 1,770,620 bytes in size.

### FIELD OF THE INVENTION

This invention pertains to optimized protein fusion linkers for creating multi-functional chimeric proteins and methods of using the same. Additionally, the invention pertains to chimeric proteins for use in guided endonuclease systems.

### BACKGROUND OF THE INVENTION

The fusion of guided endonucleases to one or more unrelated enzymes is desirable for many reasons. In some cases, it is useful to visualize and monitor successful delivery of guided endonuclease reagents into the target cell nucleus or nucleolus. In other cases, it may be useful to fuse DNA modifying enzymes to affect a specific repair outcome after guided endonuclease cleavage.

The construction of recombinant fusion protein requires the selection of a suitable linker to join the protein domains. Direct fusion of functional domains without a linker may lead to many problems, including misfolding of the fusion protein, low yield in protein production, or impaired bioactivity. For example, a barrier to using engineered nucleases fused to a fluorescent protein is that such fusions tend to have lower editing activity presumably due to steric interference caused by the unnatural fusion of the two proteins.

Achieving high functional activity for the fusion proteins can be dependent on the nature of the linker between the subunits as direct fusion of proteins can inhibit folding, stability and biological activity. Additionally, linked domains can interfere with each other's functions and physically separating these domains can lead to improved function. The nature of the linker between domains can influence average separation distances depending on linker rigidity and length.

Linkers are typically placed in three classifications: flexible, rigid, and cleavable. Flexible linkers usually consist of glycine repeats, with the periodic addition of a polar serine residue to disrupt linker-protein interaction. Rigid linkers include A(EAAAK)ₙA (SEQ ID NO: 439) repeats that form an alpha helical structure or a Pro-rich sequence, (XP)ₙ (SEQ ID NO: 440) that form relatively rigid extended rod like structures.

As such, what are needed are methods and compositions to overcome the existing challenges of current technologies. New methods and compositions for a set of universal peptide linkers that have been specifically optimized in guided endonucleases enzyme fusions is desirable to improve function of the guided endonuclease as well as the covalently fused protein partner.

### BRIEF SUMMARY OF THE INVENTION

In general, this invention pertains to methods and compositions for improved multi-functional chimeric proteins and improved linkers. In some embodiments the chimeric proteins comprise guided endonuclease proteins such as RNA guided endonucleases (“RGEN(s)”), including a Cas/CRISPR protein that are covalently fused to a partner protein with a set of universal peptide linkers. In additional embodiments, the fusion protein comprises rigid linker systems to fuse two proteins. In a further embodiment, the rigid linkers may be used for fusion proteins comprising a Cas protein fused to a fluorescent protein.

The fusion of Cas proteins of guided endonucleases to one or more unrelated protein partners is desirable for many reasons. For example, one embodiment includes the ability to generate CRISPR/Cas9 protein fusions to cleave double stranded DNA at precise locations in living cells. Cas9 is an RNA guided endonuclease from Clustered Regularly Interspaced Short Palindromic Repeat (CRISPR)-Cas (CRISPR-associated)) bacterial adaptive immune system of Streptococcus pyogenes. Cas9 is guided to a 23-nt DNA target sequence by a target site specific 20-nt complementary RNA (part of the 44-nt crRNA) and a universal 89-nt tracrRNA, collectively referred to as the guide RNA (gRNA) complex. The Cas9-gRNA ribonucleoprotein (RNP) complex mediates double-stranded DNA breaks (DSBs) which are then repaired by either the non-homologous end joining (NHEJ), which typically introduces mutations or indels at the cut site that frequently lead to gene disruption through frameshift mutation or the homology directed repair (HDR) system if a suitable template nucleic acid is present.

In one embodiment, a set of universal peptide linkers that have been specifically optimized for CRISPR enzyme fusions is desirable to improve the function of both Cas9 and the covalently-fused partner protein or protein domain. The universal linkers would not be specific to a Cas9 protein or any mutant variant thereof. In an additional embodiment, the CRISPR enzyme could be optimally linked with the universal linkers to a fluorescent protein.

In one embodiment, rigid linkers are used to covalently fuse two different proteins or protein domains. In one respect, the rigid linkers include A(EAAAK)nA (SEQ ID NO: 439) repeats that form an alpha helical structure to provide rigidity is used. In another respect, a (XP)n (SEQ ID NO: 440) repeat that forms relatively rigid extended rod like structures is used.

In a first aspect, a chimeric protein is provided. The chimeric protein includes a guided endonuclease protein, a rigid linker, and a second protein.

In a second aspect, a method of enriching cells having a chimeric protein is provided. The method includes several steps. The first step includes incubating a chimeric protein according to the first aspect with a guide RNA to form a RNP complex. The second step includes contacting the RNP complex to a plurality of target cells to produce recipient cells having the RNP complex. The third step includes sorting the recipient cells based on a fluorescence signal.

In a third aspect, an isolated nucleic acid encoding a chimeric protein is provided. The chimeric protein includes the chimeric protein of the first aspect.

In a fourth aspect, a chimeric protein is provided. The chimeric protein includes a guided endonuclease protein, a universal linker, and a second protein.

In a fifth aspect, an isolated nucleic acid is provided. The isolated nucleic acid encodes the chimeric protein according to any of respects of the fourth aspect.

In a sixth aspect, a method of enriching cells having a chimeric protein is provided. The method includes several steps. The first step includes incubating a chimeric protein according to any of the respects of the fourth aspect with a guide RNA to form a RNP complex. The second step includes contacting the RNP complex to a plurality of target cells to produce recipient cells having the RNP complex. The third step includes sorting the recipient cells based on a fluorescence signal.

### BRIEF DESCRIPTION OF THE DRAWINGS

FIG. 1A depicts percent cleavage endonuclease activity of Cas9-eGFP linker variants (L1, Cas9-GSAGSAAGSGEF (SEQ ID NO: 6)-eGFP (SEQ ID NO: 38); L2, Cas9-(AP)₇A (SEQ ID NO: 16)-eGFP (SEQ ID NO: 49); L3, Cas9-A(EAAAK)₄A (SEQ ID NO: 8)-eGFP (SEQ ID NO: 40); L4, Cas9-GGGGSEAAAKGGGG (SEQ ID NO: 430)-eGFP (SEQ ID NO: 71) and L5, Cas9-(GGGGS)₄A (SEQ ID NO: 431)-eGFP (SEQ ID NO: 39)) compared to untagged Cas9 protein (denoted as “C” (SEQ ID NO: 1)) delivered as plasmid (400 ng), with sgRNA guide targeting HPRT-38087 (SEQ ID NO: 324) delivered by reverse transfection 24 hours after plasmid delivery. The editing activity after 48 hours was measured by T7E1 assay.

FIG. 1B depicts percent cleavage endonuclease activity of Cas9-eGFP linker variants (L1, Cas9-GSAGSAAGSGEF (SEQ ID NO: 6)-eGFP (SEQ ID NO: 38); L2, Cas9-(AP)₇A-(SEQ ID NO: 16)eGFP (SEQ ID NO: 49; L3, Cas9-A(EAAAK)₄A (SEQ ID NO: 8)-eGFP (SEQ ID NO: 40); L4, Cas9-GGGGSEAAAKGGGG (SEQ ID NO: 430)-eGFP (SEQ ID NO: 71) and L5, Cas9-(GGGGS)₄A (SEQ ID NO: 431)-eGFP (SEQ ID NO: 39)) compared to untagged Cas9 protein (denoted as “C” (SEQ ID NO: 1)) delivered as plasmid (400 ng), with sgRNA guide targeting HPRT-38285 (SEQ ID NO: 326) delivered by reverse transfection 24 hours after plasmid delivery. The editing activity after 48 hours was measured by T7E1 assay.

FIG. 2 depicts percent editing efficiency of plasmid expressed Cas9-eGFP linker variants (Cas9-GSAGSAAGSGEF (SEQ ID NO: 6)-eGFP (SEQ ID NO: 38); Cas9-(AP)₇A (SEQ ID NO: 16)-eGFP (SEQ ID NO: 49); and Cas9-A(EAAAK)₄A (SEQ ID NO: 8)-eGFP (SEQ ID NO: 40)) compared to untagged Cas9 protein (SEQ ID NO: 1), with gRNA delivered into HEK293 cells by reverse transfection 24 hours after plasmid delivery. The data are displayed as box and whiskers plots showing the aggregated cleavage efficiency over 12 gRNAs that target distinct loci with HPRT (SEQ ID NOS: 319-330). Cleavage efficiency was measured with T7EI assay 48 hours after transfection.

FIG. 3A depicts editing efficiency of plasmid-expressed Cas9-eGFP linker variants (L1, Cas9-GSAGSAAGSGEF (SEQ ID NO: 6)-eGFP (SEQ ID NO: 38); L2, Cas9-(AP)₇A (SEQ ID NO: 16)-eGFP (SEQ ID NO: 49); L3, Cas9-A(EAAAK)₄A (SEQ ID NO: 8)-eGFP (SEQ ID NO: 40); L4, Cas9-A(EAAAK)₄ALEA(EAAAK)₄A (SEQ ID NO: 9)-eGFP (SEQ ID NO: 41); and L5, Cas9-LEA(EAAAK)₄ALEA(EAAAK)₄ALE (SEQ ID NO: 10)-eGFP (SEQ ID NO: 42)) compared to untagged Cas9 protein (denoted as “C,” SEQ ID NO: 1) co-delivered with an sgRNA expression plasmid into HEK293 cells by electroporation, wherein the expressed sgRNA targeted HPRT-38087 (SEQ ID NO: 324). T7E1 cleavage assays were performed on each transfected cell population 72 hours after transfection of both plasmids into the HEK293 cells.

FIG. 3B depicts editing efficiency of plasmid-expressed Cas9-eGFP linker variants (L1, Cas9-GSAGSAAGSGEF (SEQ ID NO: 6)-eGFP (SEQ ID NO: 38); L2, Cas9-(AP)₇A (SEQ ID NO: 16)-eGFP (SEQ ID NO: 49); L3, Cas9-A(EAAAK)₄A (SEQ ID NO: 8)-eGFP (SEQ ID NO: 40); L4, Cas9-A(EAAAK)₄ALEA(EAAAK)₄A (SEQ ID NO: 9)-eGFP (SEQ ID NO: 41); and L5, Cas9-LEA(EAAAK)₄ALEA(EAAAK)₄ALE (SEQ ID NO: 10)-eGFP (SEQ ID NO: 42)) compared to untagged Cas9 protein (denoted as “C,” SEQ ID NO: 1) co-delivered with an sgRNA expression plasmid into HEK293 cells by electroporation, wherein the expressed sgRNA targeted HPRT-38285 (SEQ ID NO: 326). T7E1 cleavage assay was performed 72 hours after transfection of both plasmids into the HEK293 cells.

FIG. 4A depicts editing efficiency of plasmid expressed Cas9-eGFP linker variants (1, Cas9-GSAGSAAGSGEF (SEQ ID NO: 6)-eGFP (SEQ ID NO: 38); 2, Cas9-APA-eGFP (SEQ ID NO: 43); 3, Cas9-(AP)₂A (SEQ ID NO: 11)-eGFP (SEQ ID NO: 44); 4, Cas9-(AP)₃A (SEQ ID NO: 12)-eGFP (SEQ ID NO: 45); 5, Cas9-(AP)₄A (SEQ ID NO: 13)-eGFP (SEQ ID NO: 46); 6, Cas9-(AP)₅A (SEQ ID NO: 14)-eGFP (SEQ ID NO: 47); 7, Cas9-(AP)₆A (SEQ ID NO: 15)-eGFP (SEQ ID NO: 48); 8, Cas9-(AP)₇A (SEQ ID NO: 16)-eGFP (SEQ ID NO: 49); 9, Cas9-(AP)₈A (SEQ ID NO: 17)-eGFP (SEQ ID NO: 50); 10, Cas9-(AP)₉A (SEQ ID NO: 18)-eGFP (SEQ ID NO: 51); 11, Cas9-(AP)₁₀A (SEQ ID NO: 6)-eGFP (SEQ ID NO: 52); 12, Cas9-(AP)₁₁A (SEQ ID NO: 20)-eGFP (SEQ ID NO: 53); 13, Cas9-(AP)₁₂A (SEQ ID NO: 21)-eGFP (SEQ ID NO: 54); 14, Cas9-(AP)₁₃A (SEQ ID NO: 22)-eGFP (SEQ ID NO: 55); and 15, Cas9-(AP)₁₄A (SEQ ID NO: 23)-eGFP (SEQ ID NO: 56)) compared to untagged Cas9 protein (denoted as “C,” SEQ ID NO: 1), co-delivered with sgRNA expression plasmid into HEK293 cells by electroporation, wherein the expressed sgRNA targeted HPRT-38087 (SEQ ID NO: 324). T7E1 cleavage assay was performed 72 hours after transfection of both plasmids into the HEK293 cells.

FIG. 4B depicts editing efficiency of plasmid expressed Cas9-eGFP linker variants (1, Cas9-GSAGSAAGSGEF (SEQ ID NO: 6)-eGFP (SEQ ID NO: 38); 2, Cas9-APA-eGFP (SEQ ID NO: 43); 3, Cas9-(AP)₂A (SEQ ID NO: 11)-eGFP (SEQ ID NO: 44); 4, Cas9-(AP)₃A (SEQ ID NO: 12)-eGFP (SEQ ID NO: 45); 5, Cas9-(AP)₄A (SEQ ID NO: 13)-eGFP (SEQ ID NO: 46); 6, Cas9-(AP)₅A (SEQ ID NO: 14)-eGFP (SEQ ID NO: 47); 7, Cas9-(AP)₆A (SEQ ID NO: 15)-eGFP (SEQ ID NO: 48); 8, Cas9-(AP)₇A (SEQ ID NO: 16)-eGFP (SEQ ID NO: 49); 9, Cas9-(AP)₈A (SEQ ID NO: 17)-eGFP (SEQ ID NO: 50); 10, Cas9-(AP)₉A (SEQ ID NO: 18)-eGFP (SEQ ID NO: 51); 11, Cas9-(AP)₁₀A (SEQ ID NO: 19)-eGFP (SEQ ID NO: 52); 12, Cas9-(AP)₁₁A (SEQ ID NO: 20)-eGFP (SEQ ID NO: 53); 13, Cas9-(AP)₁₂A (SEQ ID NO: 21)-eGFP (SEQ ID NO: 54); 14, Cas9-(AP)₁₃A (SEQ ID NO: 22)-eGFP (SEQ ID NO: 55); and 15, Cas9-(AP)₁₄A (SEQ ID NO: 23)-eGFP (SEQ ID NO: 56)) compared to untagged Cas9 protein (denoted as “C,” SEQ ID NO: 1), co-delivered with sgRNA expression plasmid into HEK293 cells by electroporation, wherein the expressed sgRNA targeted HPRT-38285 (SEQ ID NO: 326). T7E1 cleavage assay was performed 72 hours after transfection of both plasmids into the HEK293 cells.

FIG. 5A depicts the editing efficiency of Cas9-GFP linker variants (1, Cas9-GSAGSAAGSGEF (SEQ ID NO: 6)-eGFP (SEQ ID NO: 38); 2, Cas9-(AP)₇A (SEQ ID NO: 16)-eGFP (SEQ ID NO: 49); 3, Cas9-(AP)₉A (SEQ ID NO: 18)-eGFP (SEQ ID NO: 51); 4, Cas9-GSAGSAAGSGEF (SEQ ID NO: 6)-mCherry (SEQ ID NO: 72); 5, Cas9-(AP)₇A (SEQ ID NO: 16)-mCherry (SEQ ID NO: 83); and 6, Cas9-(AP)₉A (SEQ ID NO: 18)-mCherry (SEQ ID NO: 85)) compared to untagged Cas9 protein (denoted as “C,” SEQ ID NO: 1), delivered at a concentration of 0.0625 micromolar into HEK293 cells. The data are displayed as box and whiskers plots showing the aggregated cleavage efficiency over 12 gRNAs that target distinct loci with HPRT (SEQ ID NOS: 319-330). Cleavage efficiency was measured with T7EI assay 48 hours after transfection.

FIG. 5B depicts the editing efficiency of Cas9-GFP linker variants (1, Cas9-GSAGSAAGSGEF (SEQ ID NO: 6)-eGFP (SEQ ID NO: 38); 2, Cas9-(AP)₇A (SEQ ID NO: 16)-eGFP (SEQ ID NO: 49); 3, Cas9-(AP)₉A (SEQ ID NO: 18)-eGFP (SEQ ID NO: 51); 4, Cas9-GSAGSAAGSGEF (SEQ ID NO: 6)-mCherry (SEQ ID NO: 72); 5, Cas9-(AP)₇A (SEQ ID NO: 16)-mCherry (SEQ ID NO: 83); and 6, Cas9-(AP)₉A (SEQ ID NO: 18)-mCherry (SEQ ID NO: 85)) compared to untagged Cas9 protein (denoted as “C,” SEQ ID NO: 1), delivered at a concentration of 0.25 micromolar into HEK293 cells. The data are displayed as box and whiskers plots showing the aggregated cleavage efficiency over 12 gRNAs that target distinct loci with HPRT (SEQ ID NOS: 319-330). Cleavage efficiency was measured with T7EI assay 48 hours after transfection.

FIG. 5C depicts the editing efficiency of Cas9-GFP linker variants (1, Cas9-GSAGSAAGSGEF (SEQ ID NO: 6)-eGFP (SEQ ID NO: 38); 2, Cas9-(AP)₇A (SEQ ID NO: 16)-eGFP (SEQ ID NO: 49); 3, Cas9-(AP)₉A (SEQ ID NO: 18)-eGFP (SEQ ID NO: 51); 4, Cas9-GSAGSAAGSGEF (SEQ ID NO: 6)-mCherry (SEQ ID NO: 72); 5, Cas9-(AP)₇A (SEQ ID NO: 16)-mCherry (SEQ ID NO: 83); and 6, Cas9-(AP)₉A (SEQ ID NO: 18)-mCherry (SEQ ID NO: 85)) compared to untagged Cas9 protein (denoted as “C,” SEQ ID NO: 1), delivered at a concentration of 2.0 micromolar into HEK293 cells. The data are displayed as box and whiskers plots showing the aggregated cleavage efficiency over 12 gRNAs that target distinct loci with HPRT (SEQ ID NOS: 319-330). Cleavage efficiency was measured with T7EI assay 48 hours after transfection.

FIG. 6 depicts the effect of FACS selection of GFP signal cells on editing by Cas9-eGFP fusion proteins in HEK293 cells compared to unsorted cells. An RNP comprising chimeric Cas9-(AP)₇A (SEQ ID NO: 16)-eGFP protein (SEQ ID NO: 49) duplexed with guide RNAs was introduced into HEK293 cells by lipofection at a final concentration of 10 nM. Cells were sorted into three populations based on GFP signal corresponding to high medium and low GFP signal (high GFP signal, the top ˜20% of signal; medium GFP signal, the mid ˜80-60% of signal, and low GFP signal, the bottom ˜-60% of signal). Next Generation Sequencing (NGS) of target sites was performed for each population to measure total editing activity percentage 48-72 hours after transfection.

FIG. 7 depicts exemplary results for the editing efficiency of LbCas12a (E795L)-GFP linker variants (1, LbCas12a (E795L)-eGFP (SEQ ID NO: 314); 2, LbCas12a (E795L)-A(EAAAK)₄A (SEQ ID NO: 8)-eGFP (SEQ ID NO: 244); 3, LbCas12a (E795L)-(GGGS)₄ (SEQ ID NO: 7)-eGFP (SEQ ID NO: 243); 4, LbCas12a (E795L)-(AP)₇A (SEQ ID NO: 16)-eGFP (SEQ ID NO: 253); 5, LbCas12a (E795L)-(AP)₉A (SEQ ID NO: 18)-eGFP (SEQ ID NO: 255); and 6, LbCas12a (E795L)-A(EAAAK)₄ALEA(EAAAK)₄A (SEQ ID NO: 9)-eGFP (SEQ ID NO: 245); compared to untagged LbCas12a (E795L) protein (denoted as “C,” SEQ ID NO: 310), delivered at a concentration of 50 nM into HEK293 cells, with select ALT-R® LbCas12a crRNAs that target distinct loci with HPRT. Results shown for 38115: Cpf1 HPRT 38115-AS (SEQ ID NO: 333) and 38186: Cpf1 HPRT 38186-S(SEQ ID NO: 334); comparable results were obtained for two additional guide RNAs (Cpf1 HPRT 38330-AS (SEQ ID NO: 335) and Cpf1 HPRT 38486-S (SEQ ID NO: 336)) (data not shown).

### DETAILED DESCRIPTION OF THE INVENTION

The methods and compositions of the invention described herein provide for improved universal linkers for covalently fusing two or more proteins or protein domains. Additionally, the present invention describes methods of using the chimeric fusions. In particular, the disclosed chimeric proteins provide a robust manner whereby cells that contain the chimeric protein may be identified and sorted based upon the ability of the chimeric protein to generate a fluorescence signal. Moreover, not only do the chimeric proteins retain their enzymatic activity as guided endonucleases, but also several chimeric proteins possess surprisingly enhanced activity when compared to unmodified endonucleases or fusion proteins lacking a linker. These and other advantages of the invention, as well as additional inventive features, will be apparent from the description of the invention provided herein.

### Definitions

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but no limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term, “codon-optimized” as that term modifies “nucleic acid,” “gene,” “DNA,” or “RNA,” that encodes a protein or polypeptide, refers to a nucleic acid, gene, DNA or RNA that includes preferred codons for efficient expression of the protein or polypeptide in a given host cell or organism based upon the naturally occurring abundance of charged tRNA's specific for codons in that host cell or organism. By way of example, a codon-optimized Cas9 nucleic acid for E. coli will be suitable for optimal expression of the Cas9 protein when that nucleic acid is expressed in E coli. Similarly, a codon-optimized Cas9 nucleic acid for human cells will be suitable for optimal expression of the Cas9 protein when that nucleic acid is expressed in a human cell. Exemplary codon-optimized nucleic acids are disclosed herein. A codon-optimized nucleic acid, gene, DNA, RNA can be readily generated from a naturally occurring endogenous DNA or RNA derived from the original host cell or organism or from reverse translation of the protein amino acid sequence for the relevant protein or polypeptide sequence. One of ordinary skill in the art would understand from the literature the codon bias or preference rules for a variety of host cells or organisms. Furthermore, codon-optimized nucleic acid conversion software programs are readily available online or generally known in the art.

The terms “RNA-guided endonuclease” and “RGEN” refer to a ribonucleoprotein endonuclease that includes an RNA component for targeted enzyme activity on a given substrate. Exemplary RGEN's include CRISPR-associated endonucleases.

The term “CRISPR” refers to Clustered Regularly Interspaced Short Palindromic Repeat bacterial adaptive immune system.

The terms “Cas” and “Cas endonuclease” generally refers to a CRISPR-associated endonuclease.

The term “Cas protein” generally refers to a wild-type protein, including a variant thereof, of a CRISPR-associated endonuclease (including the interchangeable terms Cas and Cas endonuclease).

The term “Cas nucleic acid” generally refers to a nucleic acid of a CRISPR-associated endonuclease, including a guide RNA, sgRNA, crRNA, or tracrRNA.

The terms “Cas9” and “CRISPR/Cas9” refer to the CRISPR-associated bacterial adaptive immune system of Streptococcus pyogenes.

The terms “AsCas12a” and “CRISPR/AsCas12a” refer to the CRISPR-associated bacterial adaptive immune system of Acidaminococcus sp.

The terms “LbCas12a” and “CRISPR/LbCas12a” refer to the CRISPR-associated bacterial adaptive immune system of Lachnospiraceae bacterium.

The term “Cas9 protein” refers to the protein of the Cas9 or CRISPR/Cas9 endonuclease system. For the purposes of this disclosure, the wild-type Cas9 protein amino acid sequence is SEQ ID NO: 1.

The term “AsCas12a protein” refers to the protein of the AsCas12a or CRISPR/AsCas12a endonuclease system. For the purposes of this disclosure, the wild-type AsCas12a protein amino acid sequence is SEQ ID NO: 2.

The term “LbCas12a protein” refers to the protein of the LbCas12a or CRISPR/LbCas12a endonuclease system. For the purposes of this disclosure, the wild-type LbCas12a protein amino acid sequence is SEQ ID NO: 3.

The term “Cas9 nucleic acid” refers to a nucleic acid (e.g., DNA or RNA) that encodes a Cas9 protein or polypeptide. A Cas9 nucleic acid can be selected from the naturally occurring nucleic acid from Streptococcus pyogenes or a codon-optimized nucleic acid for efficient expression in a given host cell or organism. For the purposes of this disclosure, exemplary codon-optimized versions of a Cas9 nucleic acid are SEQ ID NOS: 337 and 338.

The term “AsCas12a nucleic acid” refers to a nucleic acid (e.g., DNA or RNA) that encodes a AsCas12a protein or polypeptide. An AsCas12a nucleic acid can be selected from the naturally occurring nucleic acid from Acidaminococcus sp. or a codon-optimized nucleic acid for efficient expression in a given host cell or organism. For the purposes of this disclosure, exemplary codon-optimized versions of a AsCas12a nucleic acid are SEQ ID NOS: 339 and 340.

The term “LbCas12a nucleic acid” refers to a nucleic acid (e.g., DNA or RNA) that encodes a LbCas12a protein or polypeptide. A LbCas12a nucleic acid can be selected from the naturally occurring nucleic acid from Lachnospiraceae bacterium or a codon-optimized nucleic acid for efficient expression in a given host cell or organism. For the purposes of this disclosure, exemplary codon-optimized versions of a LbCas12a nucleic acid are SEQ ID NOS: 341 and 342.

The terms “guide RNA,” “guide RNA complex” and “gRNA complex” refer to a target site-specific crRNA, a universal tracrRNA or a combination of both.

The term “sgRNA” refers to a guide RNA complex in which the crRNA is covalently linked to the tracrRNA in a single molecule.

The term “variant,” as that term modifies a protein (for example, a Cas9 protein, AsCas12a protein or LbCas12a protein), refers to a protein that includes at least one amino substitution of the reference wild-type protein amino acid sequence, additional amino acids (for example, such as an affinity tag or nuclear localization signal), or a combination thereof. An exemplary LbCas12a protein variant amino acid sequence is LbCas12a (E795L) (SEQ ID NO: 310), and chimeric proteins comprising this amino acid sequence is presented in Tables V.7, V.8 and V.10.

The term ALT-R®, as that term modifies an RNA (for example, such as a crRNA, a tracrRNA, a guide RNA, or a sgRNA), refers to an isolated, chemically-synthesized, synthetic RNA.

The term ALT-R®, as that term modifies a protein (for example, such as a Cas9 protein, an AsCas12a protein, or a LbCas12a protein), refers to an isolated, recombinant protein.

The terms “fusion protein,” “protein fusion,” “chimeric protein,” “protein chimera,” and “chimeric fusion protein” refer to a first protein or polypeptide having a covalent bond to at least one or more proteins or polypeptides, wherein the at least one or more proteins or polypeptides differ in primary sequence composition from the first protein or polypeptide. The terms fusion protein, protein fusion, chimeric protein, protein chimera and chimeric fusion protein have the same meaning and are used interchangeably. Exemplary fusion proteins are disclosed herein.

The term “universal,” as applied to modify a “guide RNA,” a “linker,” or a “linker peptide, refers to a guide RNA, linker or linker peptide for use as a guide RNA, linker or linker peptide in more than one RGEN (as related to a guide RNA) or more than one fusion protein, (as related to a linker or linker peptide). Exemplary universal linkers and universal linker peptides include flexible linkers, rigid linkers and mixed linkers, including those linkers identified in Table I and references cited therein, which are incorporated by reference in their entirety.

The term “editing activity assay” refers to an assay to determine the extent of editing of a locus by an RGEN, such as a Cas endonuclease, on at least one locus targeted by the guide RNA. Exemplary editing activity assays include those selected from a T7EI assay and a Next Generation Sequencing (NGS) assay. Exemplary T7EI assay procedures are disclosed in U.S. patent application Ser. No. 14/975,709, filed Dec. 18, 2015 (Attorney Docket IDT01-008-US), the contents of which are incorporated by reference herein. Exemplary NGS assays are disclosed in U.S. patent application Ser. No. 13/935,451, filed Jul. 3, 2013 (Attorney Docket IDT01-001-US), the contents of which are incorporated by reference herein.

The terms “linker” and “linker polypeptide” refers to a polypeptide amino acid sequence of 2 or more amino acids that covalently join a first protein or polypeptide to a second protein polypeptide. Exemplary linkers and linker polypeptides are disclosed herein.

The term “rigid linker” refers to a linker that restricts at least one degree of freedom in the motion or conformation for an affected fusion protein, protein fusion, chimeric protein, or protein chimera that includes the linker. Exemplary rigid linkers are disclosed herein.

In a first embodiment a composition for an improved multi-functional chimeric protein and improved linkers is provided. In another respect the universal peptide linker is a rigid linker system.

In another embodiment, the rigid link is used to fuse two proteins, recombinant proteins, or protein domains into a single covalently fused protein construct. In one respect the Alpha helix forming linker with the sequence of (EAAAK)n (SEQ ID NO: 433) is used. In another respect the EAAAK (SEQ ID NO: 432) amino acid sequence is repeated n times where n represents 1, 2, 3, 4, 5, 6, 7, 8, 9, 10, 11, 12, 13, or 14 repeats.

In another embodiment, the rigid linker is a Pro-rich sequence. In a further respect, the Pro-rich sequence is (XP)n (SEQ ID NO: 434) with X designating any amino acid, including preferably Ala, Lys, or Glu and more preferably Alanine. In another respect, the XP sequence is repeated n times where n represent 1, 2, 3, 4, 5, 6, 7, 8, 9, 10, 11, 12, 13, or 14 repeats. In another respect, the number of repeats is preferably 5, 6, 7, 8, or 9 repeats. In another respect the number of repeats is more preferably 7 or 9 repeats.

In another embodiment, the rigid linker is a Pro-rich sequence. In a further respect, the Pro-rich sequence is (XP)nA (SEQ ID NO: 435) with X designating any amino acid, including preferably Ala, Lys, or Glu and more preferably, Alanine and where the XP sequence is repeated n times in the range from 1-14 repeats, including n being 1, 2, 3, 4, 5, 6, 7, 8, 9, 10, 11, 12, 13, or 14 repeats. In another respect the number of repeats is preferably 5, 6, 7, 8, or 9 repeats. In another respect the number of repeats is more preferably 7 or 9 repeats.

In another embodiment, the rigid linker is used to covalently fuse a guided endonuclease and a second protein. In one respect, the rigid linker is used to covalently fuse the C-terminal end of a guided endonuclease to the N-terminal end of the second protein. Preferably, the rigid linker is used to covalently fuse the C-terminal of a RGEN to the N-terminal of a second protein. More preferably, the rigid linker is used to covalently fuse the C-terminal of a CRISPR-Cas to the N-terminal of a second protein.

In a further embodiment, the Pro-rich (XP)n (SEQ ID NO: 434) rigid linker, where n is the repeat unit in the range from 1-14 repeats including n being 1, 2, 3, 4, 5, 6, 7, 8, 9, 10, 11, 12, 13, or 14 repeats, is used to covalently fuse the C-terminal end of a guided endonuclease to the N-terminal end of a second protein. Preferably, the (XP)n (SEQ ID NO: 434) rigid linker is used to covalently fuse the C-terminal of a RGEN to the N-terminal of a second protein. More preferably, the (XP)n (SEQ ID NO: 434) rigid linker is used to covalently fuse the C-terminal of a Cas protein to the N-terminal of a second protein.

In another embodiment, the Pro-rich sequence includes a (PX)nP (SEQ ID NO: 436) rigid linker system, where n is the repeat unit in the range from 1-14 repeats including n being 1, 2, 3, 4, 5, 6, 7, 8, 9, 10, 11, 12, 13, or 14 repeats. In one respect, the Pro-rich sequence includes (PA)nP (SEQ ID NO: 437) rigid linker system where n is the repeat unit in the range from 1-14 repeats including n being 1, 2, 3, 4, 5, 6, 7, 8, 9, 10, 11, 12, 13, or 14 repeats. In another respect, the Pro-rich sequence includes (PA)₆P (SEQ ID NO: 438) rigid linker system.

In another embodiment, the rigid linker is used to covalently fuse the C-terminal end of a first protein to the N-terminal of guided endonuclease. In another respect, the rigid linker is used to covalently fuse the C-terminal of a first protein to the N-terminal of a RGEN. In another respect, the rigid linker is used to covalently fuse the C-terminal of a first protein to the N-terminal of a Cas protein.

In another embodiment, the Pro-rich (XP)n (SEQ ID NO: 434) rigid linker is used to covalently fuse the C-terminal end of a first protein to the N-terminal of guided endonuclease. In another respect, the (XP)n (SEQ ID NO: 434) linker is used to covalently fuse the C-terminal of a first protein to the N-terminal of a RGEN. In another respect the (XP)n (SEQ ID NO: 434) rigid linker is used to covalently fuse the C-terminal of a first protein to the N-terminal of a Cas protein.

In another embodiment, the CRISPR/Cas9 protein is one of the proteins covalently fused to the rigid linker. In one respect, the Cas9 protein may be a wild type Cas9 protein. In another respect the Cas protein may be a mutant or variant protein.

In another embodiment, the first protein is a guided endonuclease joined through a rigid linker to a fluorescent protein. Preferably, the guided endonuclease is an RGEN and more preferably is a CRISPR/Cas enzyme. In one respect, the fluorescent protein is eGFP or mCherry. In another aspect, the CRISPR/Cas enzyme is covalently linked to the fluorescent protein with a rigid linker to generate a CRISPR/Cas fluorescent chimeric fusion protein. The CRISPR/Cas fluorescent fusion protein allows for the ability to visualize and monitor successful delivery of CRISPR/Cas reagents into the target cell nucleus or nucleolus. In another respect, the cells may be sorted and enriched for based on the detection of the fluorescent fusion protein.

In a first aspect, a chimeric protein is provided. The chimeric protein includes a guided endonuclease protein, a rigid linker, and a second protein. In a first respect, the guided endonuclease protein is a Cas protein. In a second respect, the Cas protein is selected from the group consisting of Cas9 protein, AsCas12a protein, LbCas12a protein and a variant of Cas9 protein, AsCas12a protein, or LbCas12a protein. In a third respect, the Cas protein is selected from the group comprising SEQ ID NOS: 1, 2, and 3. In a fourth respect, the Cas protein is preferably SEQ ID NO: 1. In a fifth respect, the Cas protein is preferably SEQ ID NO: 2. In a sixth respect, the Cas protein is preferably SEQ ID NO: 3. In a seventh respect, the second protein is a fluorescent protein. In an eighth respect, the fluorescent protein is selected from the group comprising SEQ ID NOS: 4 and 5. In a ninth respect, the fluorescent protein is SEQ ID NO: 4. In a tenth respect, the fluorescent protein is SEQ ID NO: 5. In an eleventh respect, the rigid linker is selected from the group comprising XP, APA, and SEQ ID NOS: 8, 11-23 and 24-36. In a twelve respect, the rigid linker is SEQ ID NO: 8. In a thirteenth respect, the rigid linker is selected from the group comprising XP and SEQ ID NOS: 24-36. In a fourteenth respect, the rigid linker is selected from SEQ ID NOS: 29-31. In a fifteenth respect, the rigid linker is selected from the group comprising APA and SEQ ID NOS: 11-23. In a sixteenth respect, the rigid linker is selected from the group comprising APA and SEQ ID NOS: 17-18. In an eighteenth respect, the chimeric protein is selected from the group that includes SEQ ID NOS: 40-70, 74-104, 108-138, 142-172, 176-206, 210-240, 244-274, and 278-308.

In a second aspect, a method of enriching cells having a chimeric protein is provided. The method includes several steps. The first step includes incubating a chimeric protein according to the first aspect with a guide RNA to form a RNP complex. The second step includes contacting the RNP complex to a plurality of target cells to produce recipient cells having the RNP complex. The third step includes sorting the recipient cells based on a fluorescence signal. In a first respect, the method includes an additional step of performing an editing activity assay on at least one locus targeted by the guide RNA. In a second respect, the step of performing an editing activity assay on at least one locus targeted by the guide RNA is selected from an T7EI assay and a Next Generation Sequencing assay. In a third respect, the method includes a chimeric protein selected from the group consisting of SEQ ID NOS: 40-70, 74-104, 108-138, 142-172, 176-206, 210-240, 244-274, and 278-308.

In a third aspect, an isolated nucleic acid encoding a chimeric protein is provided. The chimeric protein includes the chimeric protein of the first aspect. In a first respect, the chimeric protein comprises a member selected from the group consisting of SEQ ID NOS: 40-70, 74-104, 108-138, 142-172, 176-206, 210-240, 244-274, and 278-308. In a second respect, wherein the isolated nucleic acid is codon optimized for expression in an organism or host cell. In a third respect, the organism or host cell is selected from E coli or H. sapiens.

In a fourth aspect, a chimeric protein is provided. The chimeric protein includes a guided endonuclease protein, a universal linker, and a second protein. In a first respect, the guided endonuclease protein is a Cas protein. In a second respect, the Cas protein is selected from the group consisting of Cas9 protein, AsCas12a protein, LbCas12a protein, and a variant of Cas9 protein, AsCas12a protein, and LbCas12a protein. In a third respect, the Cas protein is selected from the group comprising SEQ ID NOS: 1, 2, and 3. In a fourth respect, the Cas protein is SEQ ID NO: 1. In a fifth respect, the Cas protein is SEQ ID NO: 2. In a sixth respect, the Cas protein is SEQ ID NO: 3. In a seventh respect, the second protein is a fluorescent protein. In a eighth respect, the fluorescent protein is selected from the group comprising SEQ ID NOS: 4 and 5. In a ninth respect, the fluorescent protein is SEQ ID NO: 4. In a tenth respect, the fluorescent protein is SEQ ID NO: 5. In an eleventh respect according to any of the previous respects of the fourth aspect, the universal linker is selected from a flexible linker, a rigid linker, and a mixed linker. In a twelfth respect, the universal linker comprises a flexible linker selected from the group consisting of SEQ ID NOS: 6 and 7. In a thirteenth respect, the universal linker comprises a rigid linker selected from the group consisting of XP, APA, and SEQ ID NOS: 8-36. In a fourteenth respect, the universal linker comprises a mixed linker of SEQ ID NOS: 37. In a fifteenth respect, the chimeric protein is selected from the group consisting of SEQ ID NOS: 38-309.

In a fifth aspect, an isolated nucleic acid is provided. The isolated nucleic acid encodes the chimeric protein according to any of respects of the fourth aspect. In a first respect, the isolated nucleic acid is codon optimized for expression in an organism or host cell. In a second respect, the organism or host cell is selected from E. coli or H. sapiens. In a third respect, the isolated nucleic acid is selected from the group consisting of SEQ ID NOS: 348-387 and 389-429.

In a sixth aspect, a method of enriching cells having a chimeric protein is provided. The method includes several steps. The first step includes incubating a chimeric protein according to any of respects of the fourth aspect with a guide RNA to form a RNP complex. The second step includes contacting the RNP complex to a plurality of target cells to produce recipient cells having the RNP complex. The third step includes sorting the recipient cells based on a fluorescence signal. In a first respect, the method includes an additional step of performing an editing activity assay on at least one locus targeted by the guide RNA. In a second respect, the step of performing an editing activity assay on at least one locus targeted by the guide RNA is selected from an T7EI assay and a Next Generation Sequencing assay.

### Example 1

### Linker Effect on Editing Efficiency of Recombinant Cas9

This example identifies linkers that improve Cas9-eGFP activity. Nineteen separate plasmids that express recombinant versions of Cas9 in mammalian cells were constructed wherein the encoded protein has eGFP positioned downstream of the Cas9 carboxy-terminal domain (CTD) and the peptide sequence between Cas9 and eGFP were varied (Table I).

TABLE I
List of linker sequences.

| SEQ ID | | |
|---|---|---|
| NO: | Linker Sequence | Type |
| 6 | GSAGSAAGSGEF | Flexible¹ |
| 7 | GGGGSGGGGSGGGGSGGGGS | Flexible² |
| 8 | AEAAAKEAAAKEAAAKEAAAKA | Rigid³ |
| 9 | AEAAAKEAAAKEAAAKEAAAKALEAEAAA | Rigid³ |
| | KEAAAKEAAAKEAAAKA | |
| 10 | LEAEAAAKEAAAKEAAAKEAAAKALEAEA | Rigid³ |
| | AAKEAAAKEAAAKEAAAKALE | |
| — | APA | Rigid⁴ |
| 11 | APAPA | Rigid⁴ |
| 12 | APAPAPA | Rigid⁴ |
| 13 | APAPAPAPA | Rigid⁴ |
| 14 | APAPAPAPAPA | Rigid⁴ |
| 15 | APAPAPAPAPAPA | Rigid⁴ |
| 16 | APAPAPAPAPAPAPA | Rigid⁴ |
| 17 | APAPAPAPAPAPAPAPA | Rigid⁴ |
| 18 | APAPAPAPAPAPAPAPAPA | Rigid⁴ |
| 19 | APAPAPAPAPAPAPAPAPAPA | Rigid⁴ |
| 20 | APAPAPAPAPAPAPAPAPAPAPA | Rigid⁴ |
| 21 | APAPAPAPAPAPAPAPAPAPAPAPA | Rigid⁴ |
| 22 | APAPAPAPAPAPAPAPAPAPAPAPAPA | Rigid⁴ |
| 23 | APAPAPAPAPAPAPAPAPAPAPAPAPAPA | Rigid⁴ |
| — | XP | Rigid⁴ |
| 24 | XPXP | Rigid⁴ |
| 25 | XPXPXP | Rigid⁴ |
| 26 | XPXPXPXP | Rigid⁴ |
| 27 | XPXPXPXPXP | Rigid⁴ |
| 28 | XPXPXPXPXPXP | Rigid⁴ |
| 29 | XPXPXPXPXPXPXP | Rigid⁴ |
| 30 | XPXPXPXPXPXPXPXP | Rigid⁴ |
| 31 | XPXPXPXPXPXPXPXPXP | Rigid⁴ |
| 32 | XPXPXPXPXPXPXPXPXPXP | Rigid⁴ |
| 33 | XPXPXPXPXPXPXPXPXPXPXP | Rigid⁴ |
| 34 | XPXPXPXPXPXPXPXPXPXPXPXP | Rigid⁴ |
| 35 | XPXPXPXPXPXPXPXPXPXPXPXPXP | Rigid⁴ |
| 36 | XPXPXPXPXPXPXPXPXPXPXPXPXPXP | Rigid⁴ |
| 37 | GGGGSEAAAKGGGGS | Mixed⁵ |

¹Waldo 1999;
²Gergeron 2009;
³Bae and Shen 2006;
⁴McCormick 2001;
⁵Chen 2017

The constructs were first assayed through expression from plasmids delivered into HEK293 cells with editing assayed after 48-72 hours by T7EI digestion. Constructs showing improved activity were then subcloned into vectors for protein expression in E. coli and recombinant Cas9-fluorescent protein fusion proteins were purified by immobilized metal affinity chromatography followed by ion exchange chromatography. Purified proteins were tested for Cas9 endonuclease activity when delivered as RNP into HEK293 cells with editing assayed after 48 hours by T7 Endonuclease (T7EI) digestion.

The editing efficiency of the 19 different recombinant versions of Cas9 protein when expressed form transiently transfected plasmid in HEK293 was determined. Additionally, for a subset of the constructs were delivered as RNP. The methods and compositions disclosed herein identify linkers that result in improved editing efficiency over a baseline flexible linker design. Rigid linkers of both Pro-rich sequence (XP)n (SEQ ID NO: 434) and EAAAK (SEQ ID NO: 432) repeat varieties were found to result in an increase in Cas9 activity when delivered as plasmid. Additionally, it was found that certain (XP)n (SEQ ID NO: 434) repeat structures showed improvement in editing activity when delivered as RNP.

### Example 2

Cas9-eGFP Proteins with Rigid Linkers Expressed from Plasmids Demonstrate Improved Editing Efficiency.

This example demonstrates that introduction of rigid linkers between Cas9 and eGFP results in an improvement in Cas9 editing activity of a Cas9-eGFP fusion protein when expressed inhuman cells from transfected plasmid. The set of rigid and flexible linkers that were generate in Example 1 were further tested in the context of Cas9-linker-eGFP to assess their impact on Cas9 activity.

The editing activity of Cas9-eGFP fusion proteins containing either rigid helical [A(EAAAK)₄A](linker SEQ ID NO: 8), rigid alanine-proline [(AP)₇A](linker SEQ ID NO: 16), flexible (GGGGS)₄ (linker SEQ ID NO: 7), or a mixed flexibility linker GGGGSEAAAKGGGGS (linker SEQ ID NO: 37) were compared to wild-type Cas9 protein and the Cas9-eGFP protein with a flexible GSAGSAAGSGEF (Abbreviated GSA; linker SEQ ID NO: 6) linker serving as a baseline for comparison of editing activity. Fusion proteins were expressed from plasmids using a CMV promoter in HEK293 cells. Plasmids were delivered using the Lonza Nucleofector 96-well shuttle, using SF cell line solution, on setting DS-150 with 400 ng protein expression plasmid and 350,000 cells per well, with cells split into three wells following transfection. Two sgRNAs (HPRT-38087 (SEQ ID NO: 324) and HPRT-38285 (SEQ ID NO: 326)) targeting positions within the HPRT1 gene region were delivered at a final concentration of 30 nM by reverse transfection 24 hours after plasmid delivery using Lipofectamine RNAi Max. To analyze editing activity, genomic DNA was extracted 48 hours after reverse transfection using QuickExtract™ DNA extraction solution. Editing was assayed by PCR amplifying an ˜1 kb region of the HPRT1 gene containing target cleavage sites, melting and re-hybridizing the PCR products, digesting the products with T7EI, and quantifying the proportion of cleaved to full length sequence using the Fragment Analyzer (Agilent).

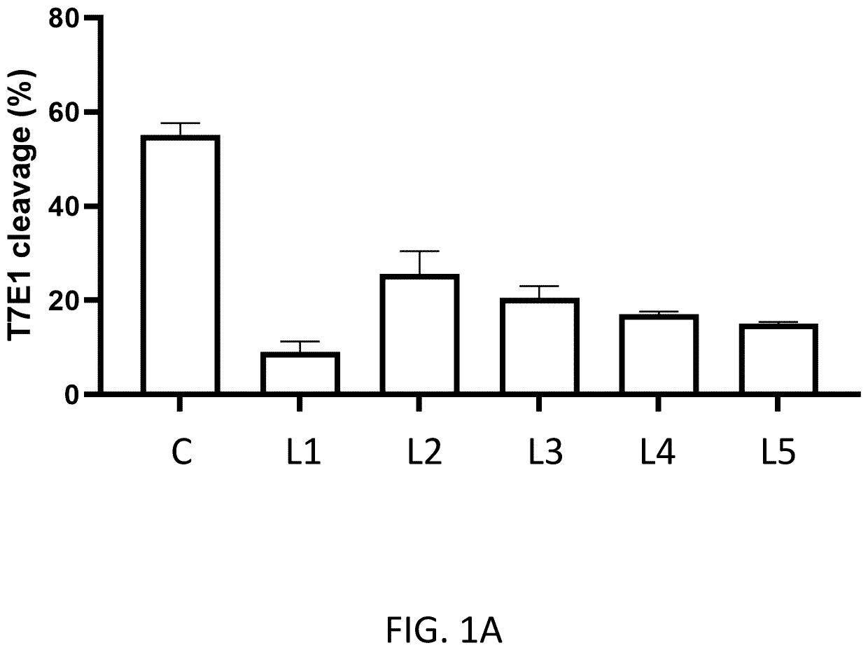

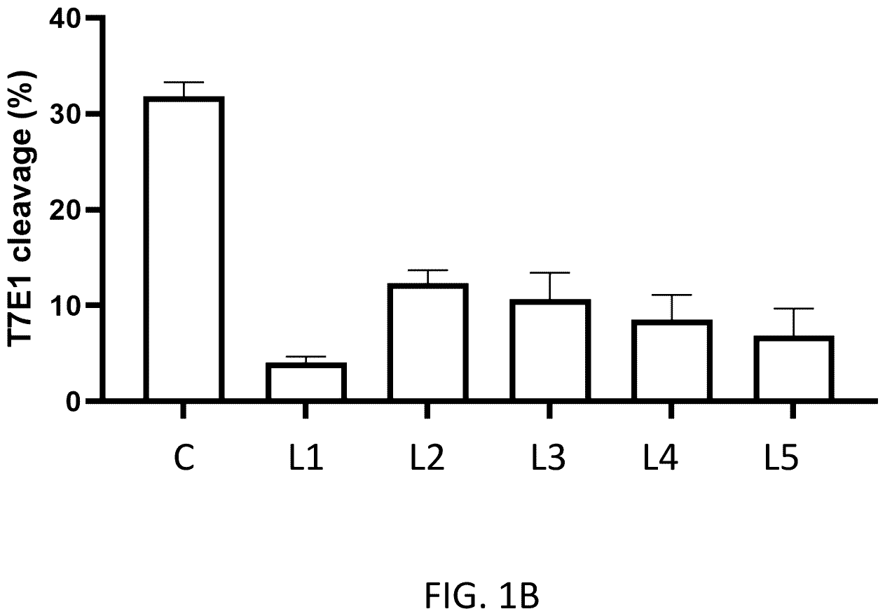

The results are shown in FIG. 1 with error bars representing the standard deviation between the three biological samples. While all four linkers resulted in improved activity over the baseline GSA linker, both types of rigid linkers resulted in an improvement in editing activity.

### Example 3

To further test rigid linkers, another experiment was performed using the same protocol as in Example 2. A set of 12 different gRNAs (Table II) targeting positions within the HPRT1 gene were tested. The PCR Primers are provided in Table III.

TABLE II
List of protospacer sequences of sgRNAs

| SEQ ID | | |
|---|---|---|
| NO: | ALT-R ® Sequence (5′→3′) | Name |
| 319 | rUrCrCrArUrUrUrCrArUrArGrUrCrU | HRPT 38094 |
| | rUrUrCrCrUrGrUrUrUrUrArGrArGrC | S-m36 |
| | rUrArUrGrCrU | |
| 320 | rUrUrUrUrGrUrArArUrUrArArCrArG | HRPT 38231 |
| | rCrUrUrGrCrGrUrUrUrUrArGrArGrC | S-m36 |
| | rUrArUrGrCrU | |
| 321 | rCrUrUrArGrArGrArArUrArUrUrUrG | HRPT 38371 |
| | rUrArGrArGrGrUrUrUrUrArGrArGrC | S-m36 |
| | rUrArUrGrCrU | |
| 322 | rUrUrGrArCrUrArUrArArUrGrArArU | HRPT 38509 |
| | rArCrUrUrCrGrUrUrUrUrArGrArGrC | S-m36 |
| | rUrArUrGrCrU | |
| 323 | rCrArArArArCrArCrArGrCrArUrArA | HRPT 38574 |
| | rArArUrUrUrGrUrUrUrUrArGrArGrC | S-m36 |
| | rUrArUrGrCrU | |
| 324 | rArArUrUrArUrGrGrGrGrArUrUrArC | HRPT 38087 |
| | rUrArGrGrArGrUrUrUrUrArGrArGrC | AS-m36 |
| | rUrArUrGrCrU | |
| 325 | rGrGrUrCrArCrUrUrUrUrArArCrArC | HRPT 38133 |
| | rArCrCrCrArGrUrUrUrUrArGrArGrC | AS-m36 |
| | rUrArUrGrCrU | |
| 326 | rCrUrUrArUrArUrCrCrArArCrArCrU | HRPT 38285 |
| | rUrCrGrUrGrGrUrUrUrUrArGrArGrC | AS-m36 |
| | rUrArUrGrCrU | |
| 327 | rGrGrCrUrUrArUrArUrCrCrArArCrA | HRPT 38287 |
| | rCrUrUrCrGrGrUrUrUrUrArGrArGrC | AS-m36 |
| | rUrArUrGrCrU | |
| 328 | rArUrUrUrCrArCrArUrArArArArCrU | HRPT 38358 |
| | rCrUrUrUrUrGrUrUrUrUrArGrArGrC | AS-m36 |
| | rUrArUrGrCrU | |
| 329 | rUrCrArArArUrUrArUrGrArGrGrUrG | HRPT 38636 |
| | rCrUrGrGrArGrUrUrUrUrArGrArGrC | AS-m36 |
| | rUrArUrGrCrU | |
| 330 | rUrArCrArGrCrUrUrUrArUrGrUrGrA | HRPT 38673 |
| | rCrUrArArUrGrUrUrUrUrArGrArGrC | AS-m36 |
| | rUrArUrGrCrU | |

TABLE III
List of Primers

| SEQ ID NO: | Sequence (5′→3′) | Name |
|---|---|---|
| 331 | AAGAATGTTGTGATAAAAGGTGAT | HRPT Low |
| | GCT | GC 7 FWD |
| 332 | ACACATCCATGGGACTTCTGCCTC | HRPT Low |
| | | GC 7 REV |

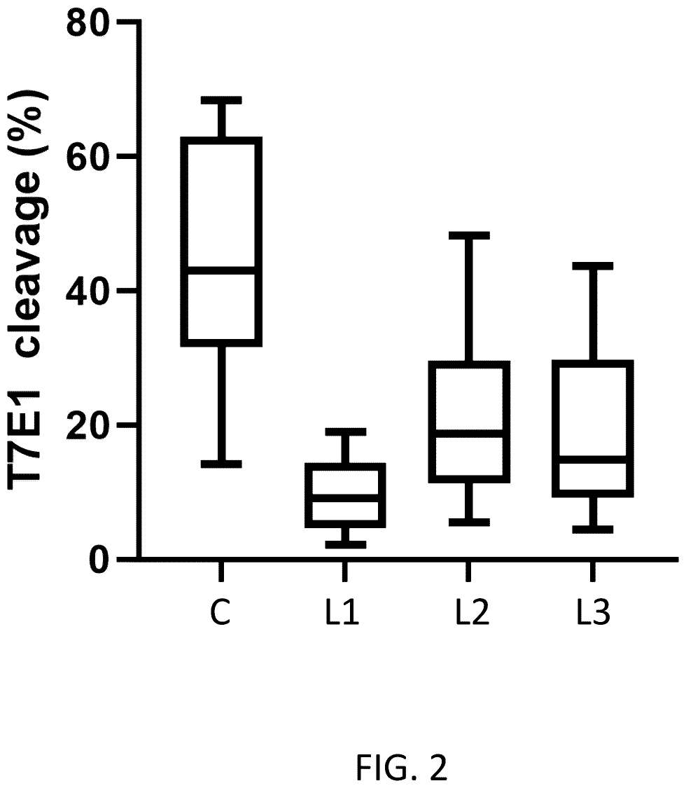

The results are shown in FIG. 2. The rigid linkers provided varying levels of improvement depending on the targeted site over the original GSA linker design, with both linkers providing improvement at each site.

### Example 4

This example demonstrates the effect of linker length by varying the number of alanine-proline repeats between 1 and 14 (APA and linker SEQ ID NOS: 11-23) and testing two different extend helical linker designs. This experiments follows the same protocol as established in Example 2; however, the guide RNA was expressed from plasmid co-delivered at 100 ng per well and the cells were allowed to grow for three days before collecting genomic DNA.

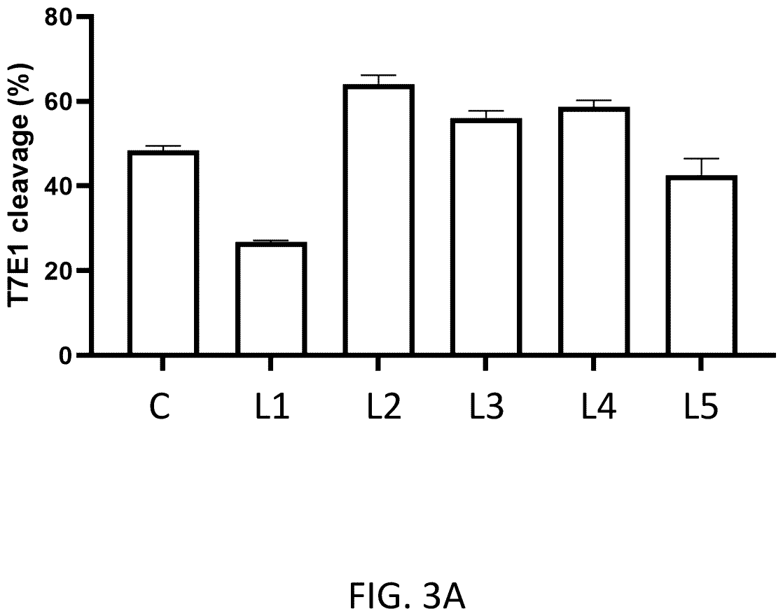

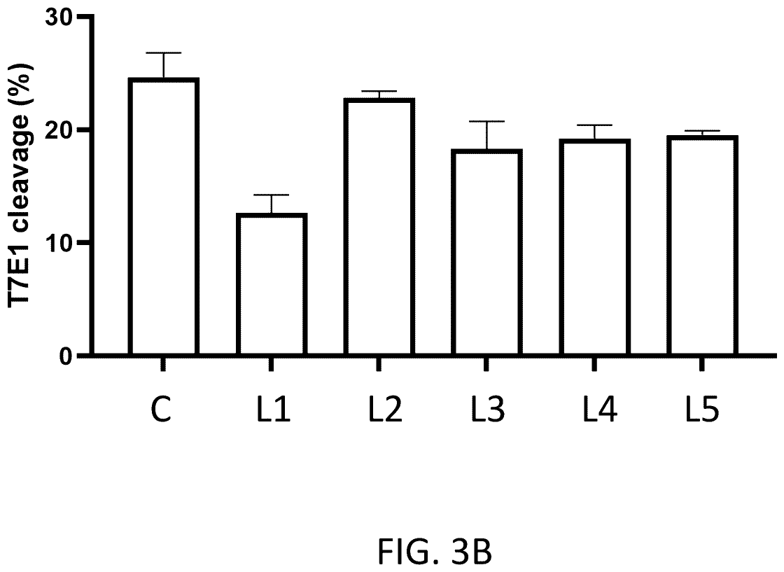

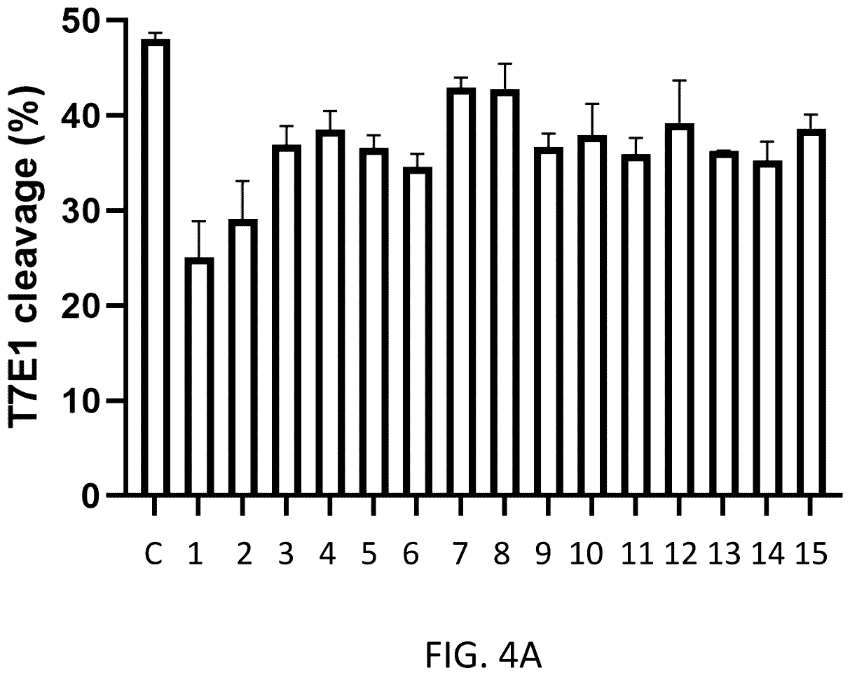

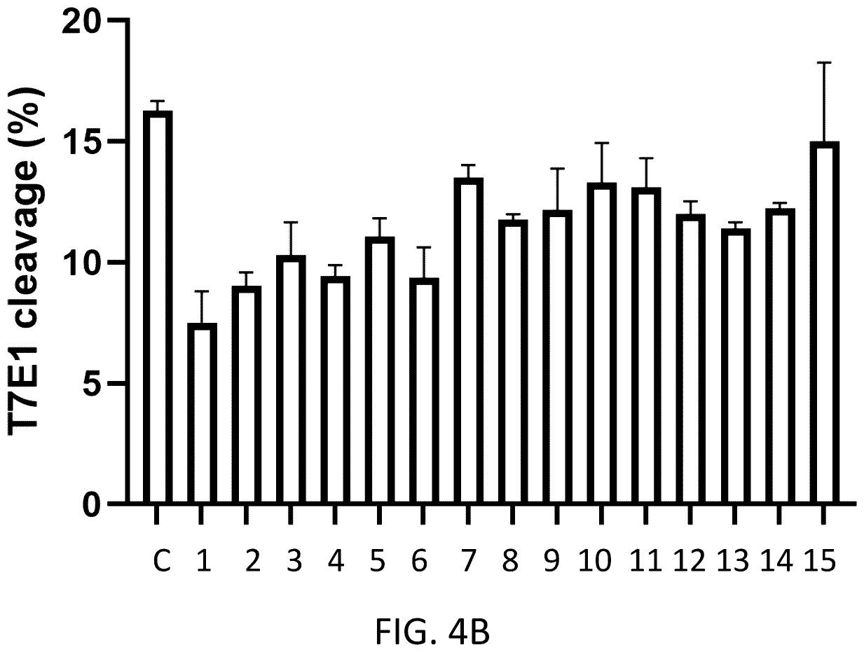

The results are shown in FIG. 3 and FIG. 4. Example 4 demonstrates that rigid helical linker length or rigid AP linker length are functional across a wide range of repeats.

### Example 5

(AP)₇A (SEQ ID NO:16) and (AP)₉A (SEQ ID NO:18) Linked Cas9-GFP and Cas9-mCherry Editing Activity when Delivered as RNP

This example demonstrates that introduction of a (AP)₇A linker (SEQ ID NO: 16) or (AP)₉A linker (SEQ ID NO: 18) between Cas9 and eGFP results in an improvement in the editing activity of a Cas9-eGFP fusion protein when delivered into human cells as RNP

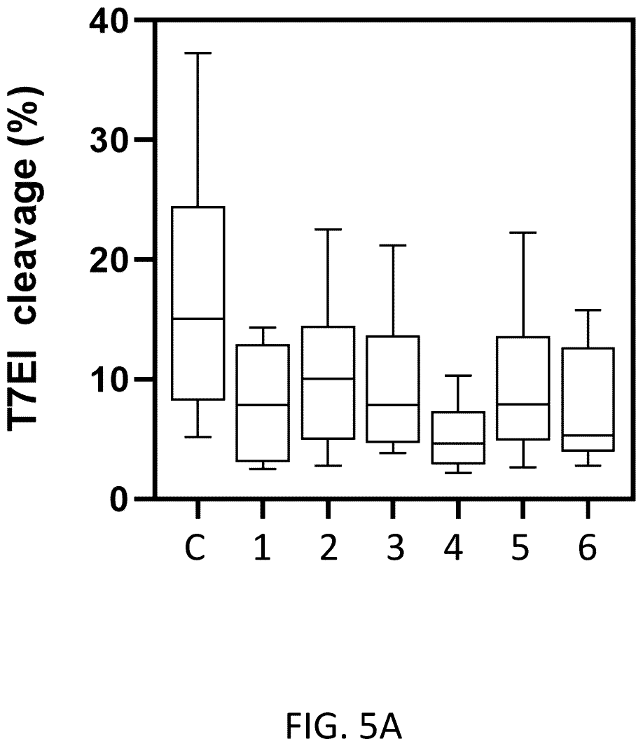

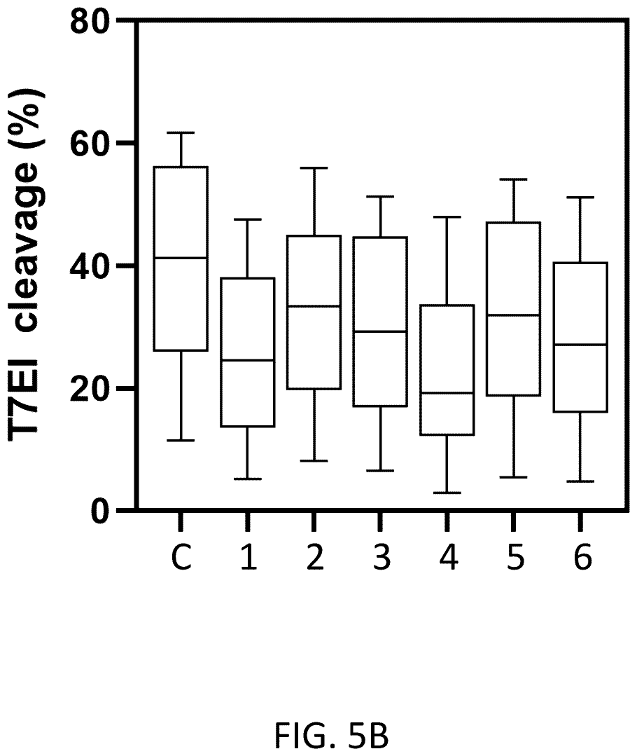

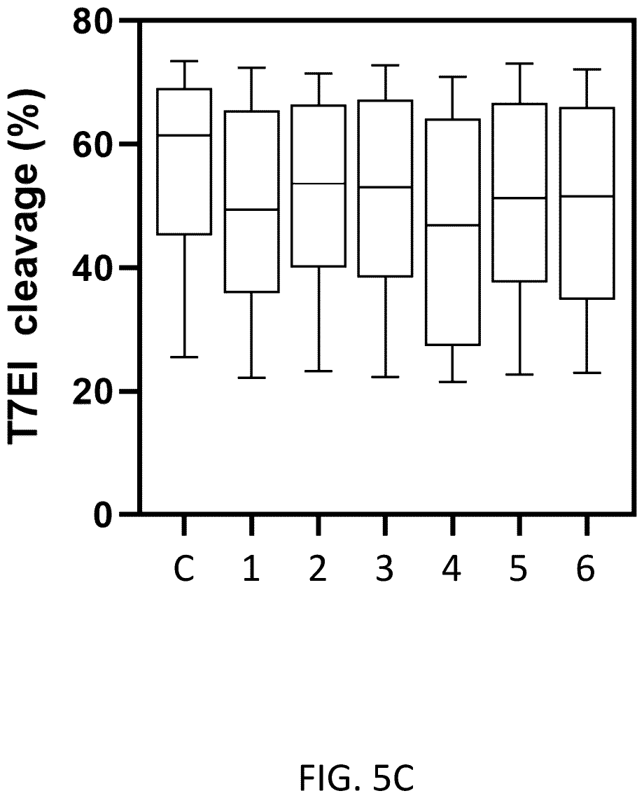

To determine whether rigid linkers increase editing activity for Cas9-GFP when delivered as RNP, Cas9-GFP linker variants containing either the GSA linker, helical rigid linker, or alanine-proline rigid linker were expressed in E coli and purified. RNP complex was formed by incubating Cas9 with ALT-R® sgRNA in PBS in a 1:1.2 ratio for 10 min and then delivered into HEK293 cells at a final RNP concentration of either 0.0625 micromolar, 0.25 micromolar, or 2.0 micromolar, along with 4 micromolar ALT-R® Cas9 Electroporation Enhancer. Editing activity was assayed by T7EI assay as previously described and the results are shown in FIG. 5.

At the 2 micromolar dose of RNP, an increase in editing activity for linker variants containing the (AP)₇A linker (linker SEQ ID NO: 16; chimeric Cas9 SEQ ID NO: 49) and (AP)₉A linker (linker SEQ ID NO: 18; chimeric Cas9 SEQ ID NO: 51) was observed compared to the flexible GSA linker (linker SEQ ID NO: 6; chimeric Cas9 SEQ ID NO: 38) over the tested sites. The benefit of these linkers was also observed for a second different fluorophore. The second fluorophore tested was mCherry, and the same (AP)ₙA (SEQ ID NO: 441) linkers were used to covalently attach the mCherry to Cas9. As seen in FIG. 5, the same rigid linkers improve Cas9 editing activity.

### Example 6

Enrichment of Cas9-eGFP Fusions with Fluorescence Activated Cell Sorting (FACS)

This example demonstrates that the use of an (AP)ₙA (SEQ ID NO: 441) linker between Cas9 and eGFP is compatible with use of Cas9-(AP)ₙA-(SEQ ID NO: 441)eGFP use with FACS to enrich for an edited cell population.

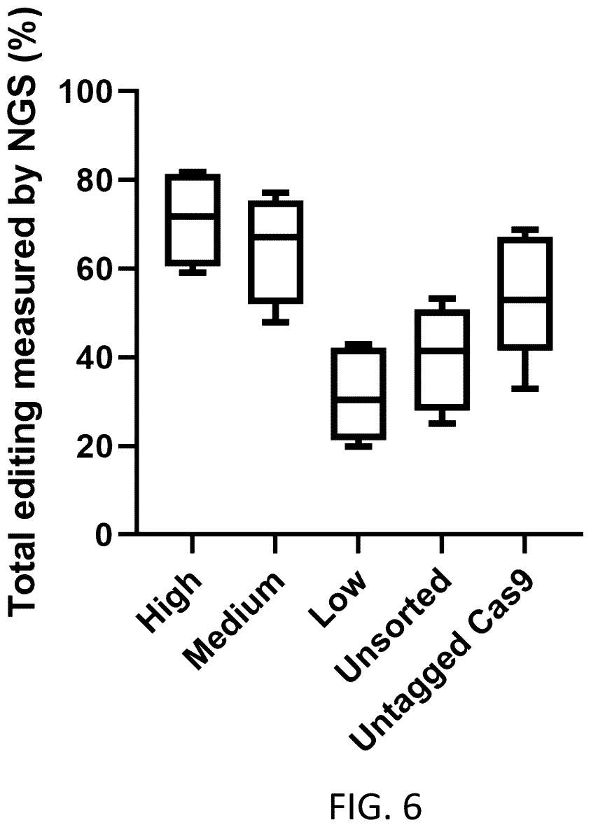

To determine whether Cas9-(AP)nA (SEQ ID NO: 441)-eGFP can be used with FACS to enrich for cells with edits, RNP complex was formed as in Example 5 using a chimeric Cas9-(AP)₇A (SEQ ID NO: 16)-eGFP protein (SEQ ID NO: 49) or wild-type Cas9 (SEQ ID NO: 1) and delivered into cells using Lipofectamine RNAiMAX at 10 nM final concentration in 3 wells of a 6 well dish with 1.2 million cells per well. After ˜16 hours, cells were trypsinized, washed with PBS, and resuspended in PBS containing 1% FBS. Cells were filtered through a 70 uM Flowmi Tip Strainer (Bel-Art). Approximately 10% of cells were transferred into a collection tube containing 500 uls of PBS with 1% FBS as the unsorted control. Cells into which Cas9-eGFP was delivered were sorted based on GFP signal using a Becton Dickinson Aria II cell sorter. Cells were sorted into three populations consisting of the cells with the top ˜20% of signal, the mid ˜80-60% of signal, and the bottom ˜60% of signal. Total editing in cells 48-72 hours after delivery was measured by NGS and results are shown in FIG. 6. The highest signal cell populations had significantly higher editing than the unsorted cell population, indicating that Cas9-eGFP fusions fused with rigid linkers is suitable for this application.

### Example 7

Evaluation of Chimeric LbCas12a Protein Variants with eGFP Protein

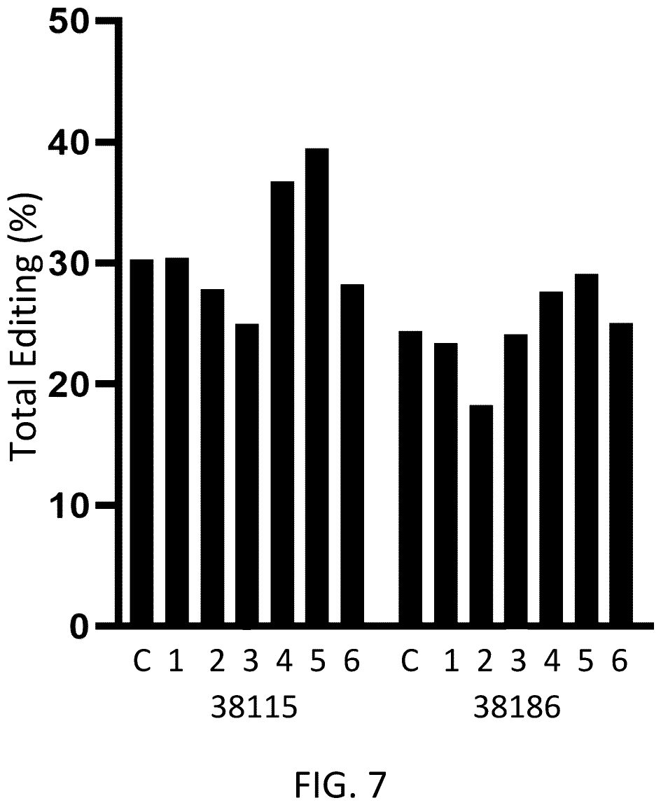

To determine whether rigid linkers increase editing efficiency in the context of another RNA guided endonuclease, the LbCas12a protein variant (E795L) (SEQ ID NO: 310) was fused to eGFP (SEQ ID NO: 4) using either no linker (chimeric LbCas12a (E795L)-eGFP protein; SEQ ID NO: 314) or a subset of the linkers listed in Table I was expressed in E coli and purified. RNP complex was formed by incubating untagged or chimeric LbCas12a (E795L) proteins with ALT-R® LbCas12a crRNA (see Table IV) in PBS in a 1:1.2 ratio for 10 min and then delivered into HEK293 cells at a final concentration of 50 nM along with 3 micromolar ALT-R® Cas12a Electroporation Enhancer. Editing 48 hours after delivery was measured by NGS and the results are shown in FIG. 7. An increase in editing activity was observed for linker variants containing the (AP)₇A (SEQ ID NO: 16) and (AP)₉A (SEQ ID NO: 18) linkers compared to no linker or the other linkers tested. These are the same linkers that were observed to provide a benefit to editing in the context of chimeric Cas9-GFP proteins.

TABLE IV
Guide RNAs used in combination with LbCas12a
protein variant (E795L) chimeras.

| SEQ ID NO: | Protospacer sequence | Guide Name |
|---|---|---|
| 333 | ACACACCCAAGGAAAGACTAT | Cpf1 HPRT 38115-AS |
| 334 | TAATGCCCTGTAGTCTCTCTG | Cpf1 HPRT 38186-S |
| 335 | GGTTAAAGATGGTTAAATGAT | Cpf1 HPRT 38330-AS |
| 336 | TTGTAGGATATGCCCTTGACT | Cpf1 HPRT 38486-S |

### Example 8

### Exemplary Sequences of Cas Proteins and Nucleic Acids

The wild-type Cas9 protein amino acid sequence is presented as SEQ ID NO: 1. Polynucleotides codon-optimized for expression in E. coli and human cells are presented in SEQ ID NOs.: 337 and 338, respectively. Exemplary variants of Cas9 protein, the polynucleotides encoding them, as well as exemplary guide RNAs are disclosed in U.S. patent application Ser. Nos. 15/729,491 and 15/964,041, filed Oct. 10, 2017 and Apr. 26, 2018, respectively (Attorney Docket Nos. IDT01-009-US and IDT01-009-US-CIP, respectively), the contents of which are incorporated by reference herein.

(Cas9 protein amino acid sequence)
SEQ ID NO: 1
MDKKYSIGLDIGTNSVGWAVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLEGNLIALSLGLTPNEKSNEDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNEDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDERKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGEDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(Cas9 DNA sequence, codon-optimized for E. coli)
SEQ ID NO: 337
ATGGGCAGCAGCGCCCCAAAGAAGAAGCGGAAGGTCGGTATCCACGGAGTCCCAGCAGCCATGGACAAAAAGTACTC
TATTGGCCTGGATATCGGGACCAACAGCGTCGGGTGGGCTGTTATCACCGACGAGTATAAAGTACCTTCGAAAAAGT
TCAAAGTGCTGGGCAACACCGATCGCCATTCAATCAAAAAGAACTTGATTGGTGCGCTGTTGTTTGACTCCGGGGAA
ACCGCCGAGGCGACTCGCCTTAAACGTACAGCACGTCGCCGGTACACTCGGCGTAAGAATCGCATTTGCTATTTGCA
GGAAATCTTTAGCAACGAGATGGCAAAAGTCGATGACTCGTTTTTCCACCGCCTCGAGGAAAGCTTTCTGGTGGAGG
AAGACAAAAAGCATGAGCGTCACCCGATCTTCGGCAACATTGTCGATGAAGTAGCGTATCATGAAAAATACCCAACC
ATTTACCACTTACGCAAAAAGCTGGTGGACAGCACTGACAAAGCTGATTTGCGCCTTATCTATTTAGCCCTGGCACA
TATGATTAAGTTTCGTGGTCACTTCCTGATCGAAGGAGACTTAAATCCCGACAACAGTGATGTTGATAAATTGTTTA
TTCAGCTTGTCCAAACTTACAATCAACTGTTCGAGGAAAACCCGATCAATGCCTCCGGTGTGGATGCAAAAGCCATT
TTAAGTGCACGCCTTAGCAAGTCCCGTCGCTTAGAAAACCTTATCGCGCAGCTGCCCGGCGAGAAAAAGAATGGTTT
GTTTGGGAACCTTATTGCCTTGAGCTTAGGCCTCACCCCGAATTTCAAAAGTAATTTCGATCTTGCAGAAGACGCCA
AATTACAACTGTCGAAGGATACTTATGATGACGATCTCGATAATCTGTTAGCGCAGATTGGTGACCAATACGCCGAT
CTTTTTCTGGCGGCTAAAAATCTGAGCGACGCCATCTTGCTTTCGGATATTCTCCGCGTTAACACCGAAATCACGAA
AGCGCCTCTTAGTGCCAGCATGATTAAACGTTATGATGAACACCACCAGGACCTGACCTTACTCAAAGCGTTGGTTC
GCCAGCAACTGCCAGAGAAGTACAAAGAAATCTTCTTTGATCAGTCAAAGAATGGTTATGCCGGCTATATTGACGGG
GGTGCAAGCCAAGAGGAATTCTACAAATTTATCAAGCCTATTCTGGAGAAAATGGATGGCACCGAAGAGTTATTGGT
GAAGCTTAACCGTGAAGACCTCCTGCGGAAACAGCGCACATTCGATAATGGTTCGATCCCACACCAAATCCATTTGG
GGGAGTTACACGCTATTTTGCGTCGCCAGGAAGACTTTTACCCTTTCCTGAAGGATAACCGGGAGAAAATTGAGAAG
ATCCTTACCTTTCGTATTCCGTATTACGTAGGCCCCTTAGCACGGGGTAATAGCCGTTTCGCGTGGATGACACGGAA
GTCGGAAGAGACGATCACCCCGTGGAACTTCGAAGAGGTAGTCGACAAGGGCGCATCAGCGCAGTCTTTTATTGAAC
GTATGACGAATTTCGATAAAAACTTGCCCAATGAGAAGGTGCTTCCGAAACATTCCTTGTTATATGAATATTTTACA
GTTTACAACGAGCTGACCAAGGTTAAATACGTGACGGAAGGAATGCGCAAGCCCGCTTTTCTTAGCGGTGAGCAAAA
AAAGGCGATCGTCGACCTGTTATTCAAAACGAATCGTAAGGTGACTGTAAAGCAACTCAAAGAAGATTACTTCAAAA
AGATTGAGTGCTTCGACAGCGTCGAAATCTCTGGGGTAGAGGATCGGTTTAACGCAAGTTTAGGTACCTACCATGAC
CTGCTTAAAATCATTAAGGATAAAGACTTCTTAGATAATGAAGAGAACGAAGATATTCTCGAGGACATCGTCTTGAC
GTTAACCTTATTTGAGGATCGTGAAATGATTGAGGAACGCCTCAAAACTTATGCCCACCTGTTCGACGATAAGGTGA
TGAAGCAGCTGAAACGTCGGCGCTACACAGGATGGGGCCGCTTGAGTCGCAAACTTATTAACGGAATCCGTGACAAG
CAATCCGGCAAAACGATTCTGGATTTCTTGAAGTCGGACGGATTTGCTAATCGCAACTTCATGCAGTTGATCCATGA
TGACTCCCTGACTTTTAAAGAGGATATTCAAAAGGCGCAGGTTAGTGGTCAAGGCGACAGCTTACACGAACACATCG
CAAATTTGGCTGGTTCGCCGGCCATTAAAAAGGGGATCCTCCAGACCGTGAAAGTTGTAGATGAGCTTGTTAAGGTC
ATGGGTCGTCATAAGCCCGAAAACATCGTGATTGAAATGGCGCGGGAGAATCAAACGACCCAGAAAGGACAAAAGAA
TAGCCGTGAACGGATGAAGCGGATCGAGGAAGGCATTAAAGAGCTGGGGTCTCAAATCTTGAAGGAACACCCTGTGG
AGAACACTCAGCTCCAAAATGAAAAACTTTACCTGTACTATTTGCAGAACGGACGCGATATGTACGTGGACCAAGAG
TTGGATATTAATCGGCTGAGTGACTACGACGTTGATCATATCGTCCCGCAGAGCTTCCTCAAAGACGATTCTATTGA
CAATAAGGTACTGACGCGCTCTGATAAAAACCGTGGTAAGTCGGACAACGTGCCCTCCGAAGAGGTTGTGAAAAAGA
TGAAAAATTATTGGCGCCAGCTTTTAAACGCGAAGCTGATCACACAACGTAAATTCGATAATTTGACCAAGGCTGAA
CGGGGTGGCCTGAGCGAGTTAGATAAGGCAGGATTTATTAAACGCCAGTTAGTGGAGACTCGTCAAATCACCAAACA
TGTCGCGCAGATTTTGGACAGCCGGATGAACACCAAGTACGATGAAAATGACAAACTGATCCGTGAGGTGAAAGTCA
TTACTCTGAAGTCCAAATTAGTTAGTGATTTCCGGAAGGACTTTCAATTCTACAAAGTCCGTGAAATTAATAACTAT
CATCACGCACATGACGCGTACCTGAATGCAGTGGTTGGGACCGCCCTTATCAAGAAATATCCTAAGCTGGAGTCGGA
GTTTGTCTATGGCGACTATAAGGTATACGATGTTCGCAAAATGATTGCGAAATCTGAGCAGGAGATCGGTAAGGCAA
CCGCAAAATATTTCTTTTACTCAAACATTATGAATTTCTTTAAGACAGAAATCACTCTGGCCAACGGGGAGATTCGC
AAACGTCCGTTGATCGAAACAAACGGCGAGACTGGCGAAATTGTTTGGGACAAAGGGCGTGATTTCGCGACGGTGCG
CAAGGTACTGAGCATGCCTCAAGTCAATATTGTTAAGAAAACCGAAGTGCAGACGGGCGGGTTTTCCAAGGAAAGCA
TCTTACCCAAACGTAATTCAGATAAACTTATTGCACGCAAAAAGGACTGGGATCCGAAAAAGTATGGAGGCTTCGAC
AGTCCAACCGTAGCCTACTCTGTTCTCGTTGTAGCGAAAGTAGAAAAGGGTAAATCCAAGAAACTGAAATCTGTCAA
GGAGTTGCTTGGAATCACCATTATGGAGCGTAGCTCCTTCGAGAAGAACCCGATTGACTTTCTGGAAGCCAAAGGAT
ATAAAGAGGTCAAGAAAGATCTTATCATTAAGCTGCCTAAGTATTCACTCTTCGAGCTGGAAAATGGTCGTAAACGC
ATGCTCGCTTCTGCCGGCGAGTTGCAGAAGGGCAATGAATTAGCACTTCCATCAAAGTACGTTAACTTCCTGTATTT
GGCCAGCCATTACGAGAAACTGAAGGGGTCTCCAGAGGACAACGAACAGAAACAATTATTTGTAGAGCAGCACAAGC
ATTATCTTGATGAAATCATTGAGCAAATTTCCGAATTCAGTAAACGCGTAATCCTGGCCGATGCAAACCTCGACAAG
GTGCTGAGCGCTTACAATAAGCATCGCGACAAACCTATCCGTGAGCAGGCTGAAAATATCATTCACCTGTTCACATT
AACGAACCTGGGCGCTCCGGCCGCTTTTAAATATTTCGACACGACAATCGACCGTAAGCGCTATACCAGTACGAAAG
AAGTGTTGGATGCGACCCTTATTCACCAGTCAATTACAGGATTATATGAGACCCGTATCGACCTTAGCCAATTAGGT
GGGGATGCGGCCCCGAAGAAAAAACGCAAAGTGGATCCGAAGAAAAAACGCAAAGTGGCGGCCGCACTCGAGCACCA
CCACCACCACCACTGA
(Cas9 DNA sequence, codon-optimized for H. sapiens)
SEQ ID NO: 338
ATGGGCAAGCCCATCCCTAACCCCCTGTTGGGGCTGGACAGCACCGCTCCCAAAAAGAAAAGGAAGGTGGGCATTCA
CGGCGTGCCTGCGGCCGACAAAAAGTACAGCATCGGCCTTGATATCGGCACCAATAGCGTGGGCTGGGCCGTTATCA
CAGACGAATACAAGGTACCCAGCAAGAAGTTCAAGGTGCTGGGGAATACAGACAGGCACTCTATCAAGAAAAACCTT
ATCGGGGCTCTGCTGTTTGACTCAGGCGAGACCGCCGAGGCCACCAGGTTGAAGAGGACCGCAAGGCGAAGGTACAC
CCGGAGGAAGAACAGGATCTGCTATCTGCAGGAGATCTTCAGCAACGAGATGGCCAAGGTGGACGACAGCTTCTTCC
ACAGGCTGGAGGAGAGCTTCCTTGTCGAGGAGGATAAGAAGCACGAACGACACCCCATCTTCGGCAACATAGTCGAC
GAGGTCGCTTATCACGAGAAGTACCCCACCATCTACCACCTGCGAAAGAAATTGGTGGATAGCACCGATAAAGCCGA
CTTGCGACTTATCTACTTGGCTCTGGCGCACATGATTAAGTTCAGGGGCCACTTCCTGATCGAGGGCGACCTTAACC
CCGACAACAGTGACGTAGACAAATTGTTCATCCAGCTTGTACAGACCTATAACCAGCTGTTCGAGGAAAACCCTATT
AACGCCAGCGGGGTGGATGCGAAGGCCATACTTAGCGCCAGGCTGAGCAAAAGCAGGCGCTTGGAGAACCTGATAGC
CCAGCTGCCCGGTGAAAAGAAGAACGGCCTCTTCGGTAATCTGATTGCCCTGAGCCTGGGCCTGACCCCCAACTTCA
AGAGCAACTTCGACCTGGCAGAAGATGCCAAGCTGCAGTTGAGTAAGGACACCTATGACGACGACTTGGACAATCTG
CTCGCCCAAATCGGCGACCAGTACGCTGACCTGTTCCTCGCCGCCAAGAACCTTTCTGACGCAATCCTGCTTAGCGA
TATCCTTAGGGTGAACACAGAGATCACCAAGGCCCCCCTGAGCGCCAGCATGATCAAGAGGTACGACGAGCACCATC
AGGACCTGACCCTTCTGAAGGCCCTGGTGAGGCAGCAACTGCCCGAGAAGTACAAGGAGATCTTTTTCGACCAGAGC
AAGAACGGCTACGCCGGCTACATCGACGGCGGAGCCAGCCAAGAGGAGTTCTACAAGTTCATCAAGCCCATCCTGGA
GAAGATGGATGGCACCGAGGAGCTGCTGGTGAAGCTGAACAGGGAAGATTTGCTCCGGAAGCAGAGGACCTTTGACA
ACGGTAGCATCCCCCACCAGATCCACCTGGGCGAGCTGCACGCAATACTGAGGCGACAGGAGGATTTCTACCCCTTC
CTCAAGGACAATAGGGAGAAAATCGAAAAGATTCTGACCTTCAGGATCCCCTACTACGTGGGCCCTCTTGCCAGGGG
CAACAGCCGATTCGCTTGGATGACAAGAAAGAGCGAGGAGACCATCACCCCCTGGAACTTCGAGGAAGTGGTGGACA
AAGGAGCAAGCGCGCAGTCTTTCATCGAACGGATGACCAATTTCGACAAAAACCTGCCTAACGAGAAGGTGCTGCCC
AAGCACAGCCTGCTTTACGAGTACTTCACCGTGTACAACGAGCTCACCAAGGTGAAATATGTGACCGAGGGCATGCG
AAAACCCGCTTTCCTGAGCGGCGAGCAGAAGAAGGCCATCGTGGACCTGCTGTTCAAGACCAACAGGAAGGTGACCG
TGAAGCAGCTGAAGGAGGACTACTTCAAGAAGATCGAGTGCTTTGATAGCGTGGAAATAAGCGGCGTGGAGGACAGG
TTCAACGCCAGCCTGGGCACCTACCACGACTTGTTGAAGATAATCAAAGACAAGGATTTCCTGGATAATGAGGAGAA
CGAGGATATACTCGAGGACATCGTGCTGACTTTGACCCTGTTTGAGGACCGAGAGATGATTGAAGAAAGGCTCAAAA
CCTACGCCCACCTGTTCGACGACAAAGTGATGAAACAACTGAAGAGACGAAGATACACCGGCTGGGGCAGACTGTCC
AGGAAGCTCATCAACGGCATTAGGGACAAGCAGAGCGGCAAGACCATCCTGGATTTCCTGAAGTCCGACGGCTTCGC
CAACCGAAACTTCATGCAGCTGATTCACGATGACAGCTTGACCTTCAAGGAGGACATCCAGAAGGCCCAGGTTAGCG
GCCAGGGCGACTCCCTGCACGAACATATTGCAAACCTGGCAGGCTCCCCTGCGATCAAGAAGGGCATACTGCAGACC
GTTAAGGTTGTGGACGAATTGGTCAAGGTCATGGGCAGGCACAAGCCCGAAAACATAGTTATAGAGATGGCCAGAGA
GAACCAGACCACCCAAAAGGGCCAGAAGAACAGCCGGGAGCGCATGAAAAGGATCGAGGAGGGTATCAAGGAACTCG
GAAGCCAGATCCTCAAAGAGCACCCCGTGGAGAATACCCAGCTCCAGAACGAGAAGCTGTACCTGTACTACCTGCAG
AACGGCAGGGACATGTACGTTGACCAGGAGTTGGACATCAACAGGCTTTCAGACTATGACGTGGATCACATAGTGCC
CCAGAGCTTTCTTAAAGACGATAGCATCGACAACAAGGTCCTGACCCGCTCCGACAAAAACAGGGGCAAAAGCGACA
ACGTGCCAAGCGAAGAGGTGGTTAAAAAGATGAAGAACTACTGGAGGCAACTGCTCAACGCGAAATTGATCACCCAG
AGAAAGTTCGATAACCTGACCAAGGCCGAGAGGGGCGGACTCTCCGAACTTGACAAAGCGGGCTTCATAAAGAGGCA
GCTGGTCGAGACCCGACAGATCACGAAGCACGTGGCCCAAATCCTCGACAGCAGAATGAATACCAAGTACGATGAGA
ATGACAAACTCATCAGGGAAGTGAAAGTGATTACCCTGAAGAGCAAGTTGGTGTCCGACTTTCGCAAAGATTTCCAG
TTCTACAAGGTGAGGGAGATCAACAACTACCACCATGCCCACGACGCATACCTGAACGCCGTGGTCGGCACCGCCCT
GATTAAGAAGTATCCAAAGCTGGAGTCCGAATTTGTCTACGGCGACTACAAAGTTTACGATGTGAGGAAGATGATCG
CTAAGAGCGAACAGGAGATCGGCAAGGCCACCGCTAAGTATTTCTTCTACAGCAACATCATGAACTTTTTCAAGACC
GAGATCACACTTGCCAACGGCGAAATCAGGAAGAGGCCGCTTATCGAGACCAACGGTGAGACCGGCGAGATCGTGTG
GGACAAGGGCAGGGACTTCGCCACCGTGAGGAAAGTCCTGAGCATGCCCCAGGTGAATATTGTGAAAAAAACTGAGG
TGCAGACAGGCGGCTTTAGCAAGGAATCCATCCTGCCCAAGAGGAACAGCGACAAGCTGATCGCCCGGAAGAAGGAC
TGGGACCCTAAGAAGTATGGAGGCTTCGACAGCCCCACCGTAGCCTACAGCGTGCTGGTGGTCGCGAAGGTAGAGAA
GGGGAAGAGCAAGAAACTGAAGAGCGTGAAGGAGCTGCTCGGCATAACCATCATGGAGAGGTCCAGCTTTGAGAAGA
ACCCCATTGACTTTTTGGAAGCCAAGGGCTACAAAGAGGTCAAAAAGGACCTGATCATCAAACTCCCCAAGTACTCC
CTGTTTGAATTGGAGAACGGCAGAAAGAGGATGCTGGCGAGCGCTGGGGAACTGCAAAAGGGCAACGAACTGGCGCT
GCCCAGCAAGTACGTGAATTTTCTGTACCTGGCGTCCCACTACGAAAAGCTGAAAGGCAGCCCCGAGGACAACGAGC
AGAAGCAGCTGTTCGTGGAGCAGCACAAGCATTACCTGGACGAGATAATCGAGCAAATCAGCGAGTTCAGCAAGAGG
GTGATTCTGGCCGACGCGAACCTGGATAAGGTCCTCAGCGCCTACAACAAGCACCGAGACAAACCCATCAGGGAGCA
GGCCGAGAATATCATACACCTGTTCACCCTGACAAATCTGGGCGCACCTGCGGCATTCAAATACTTCGATACCACCA
TCGACAGGAAAAGGTACACTAGCACTAAGGAGGTGCTGGATGCCACCTTGATCCACCAGTCCATTACCGGCCTGTAT
GAGACCAGGATCGACCTGAGCCAGCTTGGAGGCGACTCTAGGGCGGACCCAAAAAAGAAAAGGAAGGTGGAATTCCA
CCACACTGGACTAGTGGATCCGAGCTCGGTACCAAGCTTAAGTTTAAACCGCTGA

The wild-type AsCas12a protein amino acid sequence is presented as SEQ ID NO: 2. Polynucleotides codon-optimized for expression in E. coli and human cells are presented as SEQ ID NOs.: 339 and 340, respectively. Exemplary variants of AsCas12a protein, the polynucleotides encoding them, as well as exemplary guide RNAs are disclosed in U.S. patent application Ser. No. 16/536,256, filed Aug. 8, 2019, (Attorney Docket No. IDT01-013-US), the contents of which are incorporated by reference herein.

(AsCas12a protein amino acid sequence)
SEQ ID NO: 2
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGEYENRKNVESAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
(AsCas12a DNA sequence, codon-optimized for E. coli)
SEQ ID NO: 339
ATGACCCAGTTTGAAGGTTTCACCAATCTGTATCAGGTTAGCAAAACCCTGCGTTTTGAACTGATTCCGCAGGGTAA
AACCCTGAAACATATTCAAGAACAGGGCTTCATCGAAGAGGATAAAGCACGTAACGATCACTACAAAGAACTGAAAC
CGATTATCGACCGCATCTATAAAACCTATGCAGATCAGTGTCTGCAGCTGGTTCAGCTGGATTGGGAAAATCTGAGC
GCAGCAATTGATAGTTATCGCAAAGAAAAAACCGAAGAAACCCGTAATGCACTGATTGAAGAACAGGCAACCTATCG
TAATGCCATCCATGATTATTTCATTGGTCGTACCGATAATCTGACCGATGCAATTAACAAACGTCACGCCGAAATCT
ATAAAGGCCTGTTTAAAGCCGAACTGTTTAATGGCAAAGTTCTGAAACAGCTGGGCACCGTTACCACCACCGAACAT
GAAAATGCACTGCTGCGTAGCTTTGATAAATTCACCACCTATTTCAGCGGCTTTTATGAGAATCGCAAAAACGTGTT
TAGCGCAGAAGATATTAGCACCGCAATTCCGCATCGTATTGTGCAGGATAATTTCCCGAAATTCAAAGAGAACTGCC
ACATTTTTACCCGTCTGATTACCGCAGTTCCGAGCCTGCGTGAACATTTTGAAAACGTTAAAAAAGCCATCGGCATC
TTTGTTAGCACCAGCATTGAAGAAGTTTTTAGCTTCCCGTTTTACAATCAGCTGCTGACCCAGACCCAGATTGATCT
GTATAACCAACTGCTGGGTGGTATTAGCCGTGAAGCAGGCACCGAAAAAATCAAAGGTCTGAATGAAGTGCTGAATC
TGGCCATTCAGAAAAATGATGAAACCGCACATATTATTGCAAGCCTGCCGCATCGTTTTATTCCGCTGTTCAAACAA
ATTCTGAGCGATCGTAATACCCTGAGCTTTATTCTGGAAGAATTCAAATCCGATGAAGAGGTGATTCAGAGCTTTTG
CAAATACAAAACGCTGCTGCGCAATGAAAATGTTCTGGAAACTGCCGAAGCACTGTTTAACGAACTGAATAGCATTG
ATCTGACCCACATCTTTATCAGCCACAAAAAACTGGAAACCATTTCAAGCGCACTGTGTGATCATTGGGATACCCTG
CGTAATGCCCTGTATGAACGTCGTATTAGCGAACTGACCGGTAAAATTACCAAAAGCGCGAAAGAAAAAGTTCAGCG
CAGTCTGAAACATGAGGATATTAATCTGCAAGAGATTATTAGCGCAGCCGGTAAAGAACTGTCAGAAGCATTTAAAC
AGAAAACCAGCGAAATTCTGTCACATGCACATGCAGCACTGGATCAGCCGCTGCCGACCACCCTGAAAAAACAAGAA
GAAAAAGAAATCCTGAAAAGCCAGCTGGATAGCCTGCTGGGTCTGTATCATCTGCTGGACTGGTTTGCAGTTGATGA
AAGCAATGAAGTTGATCCGGAATTTAGCGCACGTCTGACCGGCATTAAACTGGAAATGGAACCGAGCCTGAGCTTTT
ATAACAAAGCCCGTAATTATGCCACCAAAAAACCGTATAGCGTCGAAAAATTCAAACTGAACTTTCAGATGCCGACC
CTGGCAAGCGGTTGGGATGTTAATAAAGAAAAAAACAACGGTGCCATCCTGTTCGTGAAAAATGGCCTGTATTATCT
GGGTATTATGCCGAAACAGAAAGGTCGTTATAAAGCGCTGAGCTTTGAACCGACGGAAAAAACCAGTGAAGGTTTTG
ATAAAATGTACTACGACTATTTTCCGGATGCAGCCAAAATGATTCCGAAATGTAGCACCCAGCTGAAAGCAGTTACC
GCACATTTTCAGACCCATACCACCCCGATTCTGCTGAGCAATAACTTTATTGAACCGCTGGAAATCACCAAAGAGAT
CTACGATCTGAATAACCCGGAAAAAGAGCCGAAAAAATTCCAGACCGCATATGCAAAAAAAACCGGTGATCAGAAAG
GTTATCGTGAAGCGCTGTGTAAATGGATTGATTTCACCCGTGATTTTCTGAGCAAATACACCAAAACCACCAGTATC
GATCTGAGCAGCCTGCGTCCGAGCAGCCAGTATAAAGATCTGGGCGAATATTATGCAGAACTGAATCCGCTGCTGTA
TCATATTAGCTTTCAGCGTATTGCCGAGAAAGAAATCATGGACGCAGTTGAAACCGGTAAACTGTACCTGTTCCAGA
TCTACAATAAAGATTTTGCCAAAGGCCATCATGGCAAACCGAATCTGCATACCCTGTATTGGACCGGTCTGTTTAGC
CCTGAAAATCTGGCAAAAACCTCGATTAAACTGAATGGTCAGGCGGAACTGTTTTATCGTCCGAAAAGCCGTATGAA
ACGTATGGCACATCGTCTGGGTGAAAAAATGCTGAACAAAAAACTGAAAGACCAGAAAACCCCGATCCCGGATACAC
TGTATCAAGAACTGTATGATTATGTGAACCATCGTCTGAGCCATGATCTGAGTGATGAAGCACGTGCCCTGCTGCCG
AATGTTATTACCAAAGAAGTTAGCCACGAGATCATTAAAGATCGTCGTTTTACCAGCGACAAATTCTTTTTTCATGT
GCCGATTACCCTGAATTATCAGGCAGCAAATAGCCCGAGCAAATTTAACCAGCGTGTTAATGCATATCTGAAAGAAC
ATCCAGAAACGCCGATTATTGGTATTGATCGTGGTGAACGTAACCTGATTTATATCACCGTTATTGATAGCACCGGC
AAAATCCTGGAACAGCGTAGCCTGAATACCATTCAGCAGTTTGATTACCAGAAAAAACTGGATAATCGCGAGAAAGA
ACGTGTTGCAGCACGTCAGGCATGGTCAGTTGTTGGTACAATTAAAGACCTGAAACAGGGTTATCTGAGCCAGGTTA
TTCATGAAATTGTGGATCTGATGATTCACTATCAGGCCGTTGTTGTGCTGGAAAACCTGAATTTTGGCTTTAAAAGC
AAACGTACCGGCATTGCAGAAAAAGCAGTTTATCAGCAGTTCGAGAAAATGCTGATTGACAAACTGAATTGCCTGGT
GCTGAAAGATTATCCGGCTGAAAAAGTTGGTGGTGTTCTGAATCCGTATCAGCTGACCGATCAGTTTACCAGCTTTG
CAAAAATGGGCACCCAGAGCGGATTTCTGTTTTATGTTCCGGCACCGTATACGAGCAAAATTGATCCGCTGACCGGT
TTTGTTGATCCGTTTGTTTGGAAAACCATCAAAAACCATGAAAGCCGCAAACATTTTCTGGAAGGTTTCGATTTTCT
GCATTACGACGTTAAAACGGGTGATTTCATCCTGCACTTTAAAATGAATCGCAATCTGAGTTTTCAGCGTGGCCTGC
CTGGTTTTATGCCTGCATGGGATATTGTGTTTGAGAAAAACGAAACACAGTTCGATGCAAAAGGCACCCCGTTTATT
GCAGGTAAACGTATTGTTCCGGTGATTGAAAATCATCGTTTCACCGGTCGTTATCGCGATCTGTATCCGGCAAATGA
ACTGATCGCACTGCTGGAAGAGAAAGGTATTGTTTTTCGTGATGGCTCAAACATTCTGCCGAAACTGCTGGAAAATG
ATGATAGCCATGCAATTGATACCATGGTTGCACTGATTCGTAGCGTTCTGCAGATGCGTAATAGCAATGCAGCAACC
GGTGAAGATTACATTAATAGTCCGGTTCGTGATCTGAATGGTGTTTGTTTTGATAGCCGTTTTCAGAATCCGGAATG
GCCGATGGATGCAGATGCAAATGGTGCATATCATATTGCACTGAAAGGACAGCTGCTGCTGAACCACCTGAAAGAAA
GCAAAGATCTGAAACTGCAAAACGGCATTAGCAATCAGGATTGGCTGGCATATATCCAAGAACTGCGTAAC
(AsCas12a DNA sequence, codon-optimized for H. sapiens)
SEQ ID NO: 340
ATGACCCAGTTTGAAGGTTTCACCAATCTGTATCAGGTTAGCAAAACCCTGCGTTTTGAACTGATTCCGCAGGGTAA
AACCCTGAAACATATTCAAGAACAGGGCTTCATCGAAGAGGATAAAGCACGTAACGATCACTACAAAGAACTGAAAC
CGATTATCGACCGCATCTATAAAACCTATGCAGATCAGTGTCTGCAGCTGGTTCAGCTGGATTGGGAAAATCTGAGC
GCAGCAATTGATAGTTATCGCAAAGAAAAAACCGAAGAAACCCGTAATGCACTGATTGAAGAACAGGCAACCTATCG
TAATGCCATCCATGATTATTTCATTGGTCGTACCGATAATCTGACCGATGCAATTAACAAACGTCACGCCGAAATCT
ATAAAGGCCTGTTTAAAGCCGAACTGTTTAATGGCAAAGTTCTGAAACAGCTGGGCACCGTTACCACCACCGAACAT
GAAAATGCACTGCTGCGTAGCTTTGATAAATTCACCACCTATTTCAGCGGCTTTTATGAGAATCGCAAAAACGTGTT
TAGCGCAGAAGATATTAGCACCGCAATTCCGCATCGTATTGTGCAGGATAATTTCCCGAAATTCAAAGAGAACTGCC
ACATTTTTACCCGTCTGATTACCGCAGTTCCGAGCCTGCGTGAACATTTTGAAAACGTTAAAAAAGCCATCGGCATC
TTTGTTAGCACCAGCATTGAAGAAGTTTTTAGCTTCCCGTTTTACAATCAGCTGCTGACCCAGACCCAGATTGATCT
GTATAACCAACTGCTGGGTGGTATTAGCCGTGAAGCAGGCACCGAAAAAATCAAAGGTCTGAATGAAGTGCTGAATC
TGGCCATTCAGAAAAATGATGAAACCGCACATATTATTGCAAGCCTGCCGCATCGTTTTATTCCGCTGTTCAAACAA
ATTCTGAGCGATCGTAATACCCTGAGCTTTATTCTGGAAGAATTCAAATCCGATGAAGAGGTGATTCAGAGCTTTTG
CAAATACAAAACGCTGCTGCGCAATGAAAATGTTCTGGAAACTGCCGAAGCACTGTTTAACGAACTGAATAGCATTG
ATCTGACCCACATCTTTATCAGCCACAAAAAACTGGAAACCATTTCAAGCGCACTGTGTGATCATTGGGATACCCTG
CGTAATGCCCTGTATGAACGTCGTATTAGCGAACTGACCGGTAAAATTACCAAAAGCGCGAAAGAAAAAGTTCAGCG
CAGTCTGAAACATGAGGATATTAATCTGCAAGAGATTATTAGCGCAGCCGGTAAAGAACTGTCAGAAGCATTTAAAC
AGAAAACCAGCGAAATTCTGTCACATGCACATGCAGCACTGGATCAGCCGCTGCCGACCACCCTGAAAAAACAAGAA
GAAAAAGAAATCCTGAAAAGCCAGCTGGATAGCCTGCTGGGTCTGTATCATCTGCTGGACTGGTTTGCAGTTGATGA
AAGCAATGAAGTTGATCCGGAATTTAGCGCACGTCTGACCGGCATTAAACTGGAAATGGAACCGAGCCTGAGCTTTT
ATAACAAAGCCCGTAATTATGCCACCAAAAAACCGTATAGCGTCGAAAAATTCAAACTGAACTTTCAGATGCCGACC
CTGGCAAGCGGTTGGGATGTTAATAAAGAAAAAAACAACGGTGCCATCCTGTTCGTGAAAAATGGCCTGTATTATCT
GGGTATTATGCCGAAACAGAAAGGTCGTTATAAAGCGCTGAGCTTTGAACCGACGGAAAAAACCAGTGAAGGTTTTG
ATAAAATGTACTACGACTATTTTCCGGATGCAGCCAAAATGATTCCGAAATGTAGCACCCAGCTGAAAGCAGTTACC
GCACATTTTCAGACCCATACCACCCCGATTCTGCTGAGCAATAACTTTATTGAACCGCTGGAAATCACCAAAGAGAT
CTACGATCTGAATAACCCGGAAAAAGAGCCGAAAAAATTCCAGACCGCATATGCAAAAAAAACCGGTGATCAGAAAG
GTTATCGTGAAGCGCTGTGTAAATGGATTGATTTCACCCGTGATTTTCTGAGCAAATACACCAAAACCACCAGTATC
GATCTGAGCAGCCTGCGTCCGAGCAGCCAGTATAAAGATCTGGGCGAATATTATGCAGAACTGAATCCGCTGCTGTA
TCATATTAGCTTTCAGCGTATTGCCGAGAAAGAAATCATGGACGCAGTTGAAACCGGTAAACTGTACCTGTTCCAGA
TCTACAATAAAGATTTTGCCAAAGGCCATCATGGCAAACCGAATCTGCATACCCTGTATTGGACCGGTCTGTTTAGC
CCTGAAAATCTGGCAAAAACCTCGATTAAACTGAATGGTCAGGCGGAACTGTTTTATCGTCCGAAAAGCCGTATGAA
ACGTATGGCACATCGTCTGGGTGAAAAAATGCTGAACAAAAAACTGAAAGACCAGAAAACCCCGATCCCGGATACAC
TGTATCAAGAACTGTATGATTATGTGAACCATCGTCTGAGCCATGATCTGAGTGATGAAGCACGTGCCCTGCTGCCG
AATGTTATTACCAAAGAAGTTAGCCACGAGATCATTAAAGATCGTCGTTTTACCAGCGACAAATTCTTTTTTCATGT
GCCGATTACCCTGAATTATCAGGCAGCAAATAGCCCGAGCAAATTTAACCAGCGTGTTAATGCATATCTGAAAGAAC
ATCCAGAAACGCCGATTATTGGTATTGATCGTGGTGAACGTAACCTGATTTATATCACCGTTATTGATAGCACCGGC
AAAATCCTGGAACAGCGTAGCCTGAATACCATTCAGCAGTTTGATTACCAGAAAAAACTGGATAATCGCGAGAAAGA
ACGTGTTGCAGCACGTCAGGCATGGTCAGTTGTTGGTACAATTAAAGACCTGAAACAGGGTTATCTGAGCCAGGTTA
TTCATGAAATTGTGGATCTGATGATTCACTATCAGGCCGTTGTTGTGCTGGAAAACCTGAATTTTGGCTTTAAAAGC
AAACGTACCGGCATTGCAGAAAAAGCAGTTTATCAGCAGTTCGAGAAAATGCTGATTGACAAACTGAATTGCCTGGT
GCTGAAAGATTATCCGGCTGAAAAAGTTGGTGGTGTTCTGAATCCGTATCAGCTGACCGATCAGTTTACCAGCTTTG
CAAAAATGGGCACCCAGAGCGGATTTCTGTTTTATGTTCCGGCACCGTATACGAGCAAAATTGATCCGCTGACCGGT
TTTGTTGATCCGTTTGTTTGGAAAACCATCAAAAACCATGAAAGCCGCAAACATTTTCTGGAAGGTTTCGATTTTCT
GCATTACGACGTTAAAACGGGTGATTTCATCCTGCACTTTAAAATGAATCGCAATCTGAGTTTTCAGCGTGGCCTGC
CTGGTTTTATGCCTGCATGGGATATTGTGTTTGAGAAAAACGAAACACAGTTCGATGCAAAAGGCACCCCGTTTATT
GCAGGTAAACGTATTGTTCCGGTGATTGAAAATCATCGTTTCACCGGTCGTTATCGCGATCTGTATCCGGCAAATGA
ACTGATCGCACTGCTGGAAGAGAAAGGTATTGTTTTTCGTGATGGCTCAAACATTCTGCCGAAACTGCTGGAAAATG
ATGATAGCCATGCAATTGATACCATGGTTGCACTGATTCGTAGCGTTCTGCAGATGCGTAATAGCAATGCAGCAACC
GGTGAAGATTACATTAATAGTCCGGTTCGTGATCTGAATGGTGTTTGTTTTGATAGCCGTTTTCAGAATCCGGAATG
GCCGATGGATGCAGATGCAAATGGTGCATATCATATTGCACTGAAAGGACAGCTGCTGCTGAACCACCTGAAAGAAA
GCAAAGATCTGAAACTGCAAAACGGCATTAGCAATCAGGATTGGCTGGCATATATCCAAGAACTGCGTAAC

The wild-type LbCas12a protein amino acid sequence is presented as SEQ ID NO: 3. Polynucleotides codon-optimized for expression in E. coli and human cells are presented in SEQ ID NOS: 341 and 342, respectively. Exemplary variants of LbCas12a protein, the polynucleotides encoding them, as well as exemplary guide RNAs are disclosed in U.S. Patent Application Ser. No. 63/018,592, filed May 1, 2020, (Attorney Docket No. IDT01-017-PRO), (now U.S. patent application Ser. No. 17/245,401, filed Apr. 30, 2021), the contents of which are incorporated by reference herein.

(LbCas12a protein amino acid sequence)
SEQ ID NO: 3
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYELHIPIAINKCPKNIFKINTEVRVLLKHDDNPYVIGIDRGERNLLYIVVVDGK
GNIVEQYSLNEIINNENGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
(LbCas12a DNA sequence, codon-optimized for E. coli)
SEQ ID NO: 341
ATGAGTAAGCTGGAGAAATTTACTAATTGTTACTCACTGTCAAAAACGCTGCGTTTCAAAGCGATCCCAGTGGGCAA
AACTCAAGAAAATATTGACAATAAACGTTTGCTTGTCGAAGACGAAAAGCGCGCCGAAGACTATAAGGGGGTGAAGA
AGCTTCTTGACCGTTACTACCTTTCATTCATCAACGATGTTCTGCACAGCATCAAGTTAAAAAACCTTAACAATTAC
ATTAGTTTGTTTCGCAAGAAGACACGTACAGAGAAAGAGAATAAAGAGCTTGAAAACTTAGAGATTAACCTTCGCAA
GGAGATCGCAAAGGCGTTCAAAGGCAATGAAGGCTATAAGTCGTTATTTAAAAAAGACATCATTGAAACGATTCTGC
CCGAGTTTTTGGATGATAAGGATGAAATCGCCTTAGTTAATTCGTTCAATGGGTTCACTACAGCCTTTACAGGATTC
TTTGATAACCGTGAAAACATGTTTAGCGAGGAAGCAAAAAGCACGAGCATTGCATTTCGCTGCATTAACGAAAATTT
GACTCGTTATATCTCTAACATGGACATCTTTGAGAAGGTTGACGCGATCTTCGATAAGCACGAAGTCCAGGAGATCA
AGGAGAAGATTCTGAACAGCGATTACGACGTTGAAGACTTTTTCGAGGGGGAATTTTTTAACTTTGTGCTTACGCAG
GAAGGAATCGATGTATATAATGCAATTATTGGTGGCTTCGTGACAGAGTCAGGTGAGAAGATTAAAGGATTAAATGA
ATATATCAATTTATACAATCAGAAGACCAAGCAGAAGCTTCCCAAGTTCAAGCCACTGTACAAGCAAGTGTTGTCCG
ACCGCGAGAGCCTTTCCTTTTACGGTGAAGGCTATACCAGTGACGAAGAGGTGTTAGAAGTCTTCCGTAACACGTTA
AACAAAAATTCCGAAATTTTCTCATCCATCAAAAAGCTGGAGAAATTATTTAAGAACTTCGACGAATACAGTAGTGC
AGGGATTTTTGTGAAAAATGGACCAGCTATCAGTACTATTTCTAAAGACATCTTCGGGGAATGGAATGTGATTCGCG
ATAAGTGGAATGCTGAGTATGATGATATTCATCTGAAGAAGAAGGCTGTCGTAACGGAGAAGTATGAGGACGATCGC
CGTAAGTCATTCAAAAAAATTGGCTCCTTCAGCCTTGAGCAGCTGCAGGAGTATGCTGATGCTGATCTGTCGGTTGT
CGAGAAGTTAAAAGAGATTATTATCCAAAAGGTAGACGAAATTTATAAGGTCTACGGGAGTAGCGAAAAGTTGTTTG
ATGCTGACTTCGTTTTAGAAAAAAGTTTGAAGAAGAATGATGCGGTCGTTGCCATTATGAAAGATTTGCTGGATAGT
GTTAAATCTTTCGAGAATTACATCAAAGCCTTTTTTGGTGAAGGGAAAGAGACTAATCGCGACGAGTCTTTCTACGG
AGATTTTGTGTTAGCGTACGATATCCTGTTGAAGGTAGACCACATCTATGACGCTATTCGCAATTATGTCACACAAA
AACCATATTCTAAGGACAAGTTCAAATTGTACTTTCAGAACCCCCAATTCATGGGCGGATGGGACAAGGATAAGGAA
ACAGACTACCGTGCGACGATTTTGCGCTACGGTAGCAAATACTACCTGGCTATCATGGACAAAAAGTACGCAAAATG
TTTACAGAAAATTGACAAGGATGACGTCAACGGTAACTATGAAAAAATCAATTACAAATTACTGCCTGGGCCAAATA
AGATGCTGCCAAAAGTGTTCTTTAGTAAGAAATGGATGGCCTACTATAATCCGTCTGAGGACATCCAAAAAATCTAC
AAAAACGGCACGTTCAAGAAGGGAGATATGTTCAATTTAAACGATTGTCACAAATTAATCGATTTTTTTAAAGATTC
CATTTCCCGTTATCCAAAGTGGTCCAATGCATATGACTTCAACTTTAGCGAGACCGAGAAATACAAGGATATCGCCG
GATTCTATCGTGAGGTGGAGGAACAAGGTTACAAGGTCTCCTTTGAGTCAGCCTCTAAAAAGGAGGTTGATAAATTG
GTGGAGGAGGGCAAATTGTATATGTTCCAAATTTACAACAAAGACTTCTCTGACAAGAGTCATGGCACTCCTAACTT
GCACACTATGTATTTCAAATTGTTATTTGATGAAAATAATCATGGTCAAATCCGTTTGTCAGGCGGTGCAGAATTGT
TCATGCGTCGCGCTAGCTTAAAGAAGGAGGAGTTAGTAGTACACCCGGCTAACAGTCCGATCGCTAACAAAAACCCG
GACAATCCCAAAAAAACAACTACGCTGAGTTACGACGTGTATAAAGATAAACGCTTCTCAGAGGACCAATATGAACT
GCATATCCCCATCGCCATCAATAAGTGCCCCAAGAACATTTTCAAGATCAATACGGAAGTACGCGTCCTGTTGAAGC
ACGATGATAACCCGTACGTCATTGGTATCGACCGCGGTGAACGCAATTTACTTTATATCGTCGTAGTGGACGGTAAA
GGCAACATTGTAGAGCAATATTCCTTAAACGAGATCATCAATAATTTTAACGGCATCCGCATTAAAACAGATTACCA
CTCTCTGCTGGATAAAAAAGAGAAAGAGCGTTTCGAAGCTCGTCAGAATTGGACCTCCATCGAGAATATCAAAGAGC
TTAAAGCGGGGTACATTTCACAGGTGGTGCATAAGATTTGCGAGTTGGTAGAAAAATACGACGCCGTAATCGCATTG
GAGGATCTGAATTCGGGTTTCAAAAACAGCCGTGTTAAGGTTGAAAAACAAGTCTATCAAAAATTCGAAAAGATGTT
AATTGACAAATTGAACTATATGGTTGATAAAAAGTCGAATCCATGTGCGACAGGGGGGGCATTAAAGGGATATCAGA
TTACTAATAAATTCGAATCGTTCAAGAGCATGAGCACTCAAAATGGCTTCATCTTTTACATCCCAGCATGGCTGACC
TCGAAAATTGATCCTTCCACAGGTTTCGTCAACCTTTTGAAGACTAAGTATACTAGTATTGCGGACTCGAAGAAGTT
CATCAGTTCTTTCGACCGTATTATGTATGTGCCGGAGGAGGATCTGTTTGAGTTCGCATTGGATTATAAGAATTTCA
GCCGCACCGACGCGGACTACATTAAAAAATGGAAATTATACTCATACGGAAATCGCATTCGTATCTTTCGTAATCCC
AAAAAGAACAATGTTTTCGACTGGGAGGAAGTGTGCCTTACTTCGGCGTATAAGGAGCTTTTCAACAAATACGGAAT
TAACTACCAGCAGGGCGATATTCGCGCCCTGTTATGTGAACAATCTGATAAAGCGTTTTACAGCTCCTTTATGGCAT
TAATGTCCTTAATGTTGCAGATGCGCAACTCGATCACGGGTCGTACAGACGTTGATTTCTTAATCTCACCGGTTAAG
AACAGTGACGGCATCTTCTACGACTCTCGCAACTACGAGGCACAGGAGAATGCCATCTTACCCAAGAACGCAGATGC
AAATGGTGCGTACAATATCGCTCGTAAAGTCCTTTGGGCAATCGGCCAATTCAAGAAGGCTGAAGACGAGAAGCTGG
ACAAGGTGAAAATCGCGATTTCTAATAAGGAATGGCTGGAGTATGCGCAGACGAGTGTAAAGCAC
(LbCas12a DNA sequence, codon-optimized for H. sapiens)
SEQ ID NO: 342
ATGTCTAAACTGGAGAAATTCACTAATTGCTACTCCTTGTCAAAAACATTGCGATTTAAAGCTATACCCGTTGGTAA
GACTCAGGAAAACATCGACAACAAACGGCTTCTGGTAGAGGACGAAAAACGCGCCGAGGATTATAAAGGAGTAAAAA
AACTGTTGGACAGGTACTACCTGAGTTTCATCAATGACGTGCTGCATAGTATTAAACTGAAAAACCTCAACAATTAC
ATCTCTCTGTTTAGAAAAAAGACACGGACGGAAAAGGAGAATAAAGAATTGGAGAATCTTGAGATAAACCTGCGCAA
GGAAATAGCCAAGGCATTCAAAGGAAACGAAGGCTACAAGTCCTTGTTTAAAAAGGATATTATCGAGACCATACTTC
CCGAGTTTTTGGACGACAAAGACGAAATCGCTCTGGTGAACTCTTTTAATGGCTTTACTACAGCCTTTACAGGATTT
TTTGACAACAGAGAGAATATGTTCTCTGAGGAGGCAAAGTCTACCAGCATTGCCTTCCGATGTATAAACGAAAACCT
GACAAGATATATATCAAACATGGACATTTTTGAAAAAGTTGATGCGATATTTGACAAACACGAGGTCCAAGAGATAA
AGGAAAAAATCCTGAACTCAGATTACGACGTGGAAGATTTTTTTGAGGGTGAGTTTTTCAATTTCGTTCTTACGCAG
GAGGGGATCGACGTATATAACGCTATCATTGGGGGCTTCGTTACCGAATCTGGAGAAAAGATCAAAGGCCTTAACGA
GTATATAAATCTGTACAATCAGAAAACGAAGCAAAAGCTCCCAAAGTTCAAGCCCCTTTATAAACAGGTCCTTAGTG
ACCGGGAAAGCCTCTCTTTCTATGGAGAAGGGTACACCTCCGATGAAGAGGTTCTGGAGGTGTTCCGAAATACTCTC
AATAAAAACTCTGAAATTTTCAGCAGCATCAAAAAATTGGAGAAGCTCTTTAAAAATTTCGACGAATATTCCTCTGC
TGGAATATTTGTGAAGAACGGACCCGCAATCTCTACCATAAGCAAGGATATCTTTGGTGAATGGAATGTAATTAGAG
ATAAATGGAATGCGGAGTACGATGACATCCACCTCAAAAAAAAGGCTGTCGTAACGGAGAAATACGAAGATGACCGC
AGAAAGAGTTTCAAAAAAATCGGTTCTTTTTCACTGGAACAGCTCCAGGAATACGCGGATGCCGACTTGAGCGTTGT
AGAAAAACTGAAAGAAATAATTATCCAGAAAGTAGATGAGATTTACAAAGTGTATGGGAGCAGCGAAAAACTTTTTG
ATGCAGATTTTGTACTTGAAAAAAGTCTTAAAAAAAATGATGCAGTTGTCGCCATTATGAAAGATCTCCTCGACAGT
GTAAAATCTTTTGAGAATTACATCAAGGCTTTTTTTGGCGAGGGCAAGGAGACGAATAGGGATGAAAGTTTTTATGG
TGACTTTGTACTTGCTTACGACATCCTGCTCAAGGTCGATCATATTTACGATGCTATACGCAATTACGTGACACAGA
AACCGTACTCTAAAGATAAGTTTAAGTTGTATTTCCAGAATCCTCAGTTCATGGGTGGCTGGGACAAGGATAAGGAG
ACAGATTATCGAGCGACTATTTTGCGCTATGGCTCTAAGTATTACCTCGCTATAATGGACAAAAAGTACGCTAAATG
CCTGCAGAAAATTGATAAGGACGACGTGAACGGTAACTATGAGAAAATTAACTATAAGTTGTTGCCCGGCCCTAATA
AGATGCTGCCGAAAGTCTTTTTCTCCAAAAAGTGGATGGCGTACTACAATCCATCAGAAGATATCCAGAAGATATAC
AAGAATGGCACCTTTAAGAAAGGAGATATGTTTAACCTTAACGACTGTCACAAGTTGATTGACTTCTTTAAGGATAG
CATTTCTCGATACCCAAAGTGGTCTAATGCATATGATTTCAATTTCTCTGAGACGGAGAAGTATAAAGACATTGCGG
GGTTCTACCGAGAAGTCGAAGAACAAGGGTACAAGGTAAGTTTTGAATCTGCTTCAAAAAAAGAGGTAGACAAACTG
GTAGAAGAGGGAAAACTCTACATGTTCCAGATCTATAATAAGGACTTCTCTGACAAAAGTCACGGTACTCCGAACCT
CCATACGATGTACTTCAAGCTGCTTTTTGATGAAAACAATCATGGCCAGATACGCCTCAGTGGAGGAGCGGAATTGT
TTATGCGCCGCGCCTCCCTGAAGAAAGAAGAGCTGGTAGTACATCCTGCCAATTCCCCAATCGCAAACAAAAATCCG
GATAATCCTAAAAAGACCACTACGTTGAGCTACGACGTTTACAAGGATAAGAGATTTTCCGAGGATCAGTACGAGCT
TCATATCCCTATCGCCATAAATAAGTGCCCAAAGAATATTTTCAAAATAAACACGGAAGTAAGAGTACTGCTCAAGC
ATGATGATAATCCCTACGTAATTGGAATTGATAGGGGGGAACGGAACTTGCTTTATATAGTCGTCGTCGATGGCAAA
GGGAACATTGTTGAACAATACTCTCTGAATGAGATCATCAACAACTTCAACGGTATCCGCATTAAAACTGATTATCA
TTCTCTCCTGGACAAAAAAGAAAAGGAAAGGTTCGAAGCGCGCCAGAACTGGACTTCAATTGAAAACATTAAGGAGC
TCAAAGCTGGCTATATATCTCAGGTTGTCCATAAGATATGCGAACTGGTAGAAAAATATGATGCCGTCATTGCTCTC
GAGGACCTGAACAGCGGTTTTAAGAACAGCCGGGTCAAAGTCGAAAAGCAGGTCTATCAAAAGTTCGAGAAGATGCT
CATAGACAAACTCAACTATATGGTCGATAAGAAGTCTAACCCGTGTGCTACGGGAGGGGCACTGAAGGGATACCAGA
TCACGAATAAGTTTGAATCATTCAAAAGCATGAGTACCCAAAATGGATTTATTTTCTACATTCCCGCGTGGTTGACC
TCAAAAATCGACCCTAGTACAGGATTCGTGAACCTGCTGAAGACTAAGTACACTAGTATTGCGGATAGCAAGAAATT
TATAAGCTCTTTTGACCGCATTATGTACGTACCCGAAGAAGATTTGTTTGAGTTCGCTCTCGACTACAAAAATTTTA
GCAGAACCGACGCCGATTACATAAAGAAGTGGAAACTCTACTCTTATGGGAACCGCATACGCATATTCCGAAACCCT
AAAAAAAACAATGTATTCGATTGGGAAGAGGTTTGCCTGACCAGCGCGTACAAAGAGCTTTTTAACAAGTATGGTAT
AAATTATCAGCAGGGTGACATCCGCGCCTTGTTGTGTGAGCAAAGTGACAAGGCGTTCTACAGTTCATTTATGGCGT
TGATGTCCCTCATGTTGCAGATGCGAAATTCCATCACGGGGAGGACAGATGTAGATTTCCTCATCTCTCCGGTCAAG
AACAGTGATGGCATCTTTTACGACAGCCGGAATTACGAGGCGCAGGAGAATGCCATACTCCCTAAGAATGCGGACGC
GAACGGAGCATATAACATCGCGCGAAAAGTCCTGTGGGCTATTGGTCAATTCAAAAAGGCTGAAGATGAGAAGTTGG
ACAAGGTGAAGATAGCCATTAGTAACAAGGAATGGTTGGAGTACGCTCAGACATCAGTGAAACAT

### Example 9

### Sequences of Fluorescent Proteins

(eGFP protein amino acid sequence) (Cormack et
al. (1996)).
SEQ ID NO: 4
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK
eGFP DNA sequence
(codon-optimized for E. coli)
SEQ ID NO: 343
ATGGTAAGTAAAGGTGAAGAGCTTTTTACAGGTGTGGTGCCCATCCTTG
TGGAGTTGGACGGTGATGTCAATGGCCATAAATTTTCTGTGTCTGGTGA
AGGGGAAGGGGACGCGACGTATGGAAAACTTACCCTGAAGTTTATCTGC
ACGACAGGTAAGTTGCCAGTACCGTGGCCTACCCTGGTCACCACATTAA
CATATGGTGTTCAATGCTTTTCACGCTACCCTGACCACATGAAACAACA
TGACTTTTTTAAAAGTGCCATGCCAGAGGGCTACGTGCAGGAACGCACA
ATCTTTTTCAAAGACGACGGCAATTATAAAACACGCGCGGAGGTAAAGT
TTGAGGGAGACACACTGGTTAATCGCATCGAACTGAAAGGCATTGACTT
TAAGGAGGATGGGAATATCTTAGGCCATAAACTGGAGTATAACTATAAC
TCTCACAACGTCTATATTATGGCGGACAAGCAAAAGAATGGTATCAAGG
TAAACTTTAAGATTCGTCATAACATTGAGGATGGGAGCGTGCAGTTGGC
TGACCACTATCAGCAGAATACTCCCATTGGCGACGGCCCCGTGCTTTTA
CCTGACAATCACTATCTTTCTACGCAGTCAGCTTTGTCCAAAGACCCCA
ACGAGAAGCGTGATCACATGGTTCTTCTGGAATTCGTCACAGCCGCCGG
AATCACTTTAGGCATGGACGAACTTTATAAA
eGFP DNA sequence
(codon-optimized for H. sapiens)
SEQ ID NO: 344
ATGGTCTCAAAAGGCGAAGAATTGTTCACCGGTGTAGTCCCTATCTTGG
TGGAGCTTGACGGCGATGTTAATGGCCATAAATTTAGTGTGTCCGGCGA
AGGTGAGGGCGACGCCACATATGGTAAACTTACGCTTAAATTTATTTGC
ACGACGGGAAAGCTCCCCGTGCCGTGGCCAACCCTTGTAACTACCCTTA
CGTACGGGGTGCAGTGCTTCAGTAGGTATCCCGACCATATGAAGCAGCA
CGATTTTTTCAAAAGTGCTATGCCCGAGGGGTATGTGCAAGAGAGGACT
ATATTCTTTAAGGATGATGGGAATTACAAGACGCGAGCCGAGGTTAAGT
TCGAAGGTGATACGCTTGTAAATCGAATCGAATTGAAAGGGATTGACTT
TAAGGAAGACGGAAATATACTTGGACACAAATTGGAATATAACTACAAC
AGCCACAACGTCTATATAATGGCCGACAAGCAAAAGAACGGAATAAAAG
TCAACTTTAAGATTCGACACAATATAGAGGATGGATCCGTGCAGCTTGC
TGACCACTATCAGCAGAACACTCCGATAGGCGATGGACCAGTGCTGCTT
CCCGACAATCACTACCTCTCCACGCAGTCTGCTCTGTCTAAAGACCCTA
ATGAAAAACGAGACCACATGGTGCTGCTCGAATTTGTCACTGCCGCCGG
GATTACCTTGGGAATGGACGAACTGTATAAG
(mCherry amino acid sequence) (Shaner et
al. (12004).
SEQ ID NO: 5
MVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMG
WEASSERMYPEDGALKGEIKQRLKLKDGGHYDAEVKTTYKAKKPVQLPG
AYNVNIKLDITSHNEDYTIVEQYERAEGRHSTGGMDELYK
(mCherry DNA sequence codon-optimized
for E. coli)
SEQ ID NO: 345
ATGGTGTCCAAAGGAGAAGAGGATAACATGGCGATCATCAAGGAATTTA
TGCGTTTCAAAGTTCACATGGAGGGAAGCGTAAACGGCCATGAATTTGA
GATTGAGGGGGAAGGCGAAGGCCGTCCATACGAAGGCACTCAAACCGCC
AAGCTTAAGGTTACCAAGGGAGGTCCGCTTCCTTTCGCCTGGGATATCC
TTAGCCCACAGTTCATGTATGGCTCTAAAGCATACGTCAAGCACCCTGC
AGACATTCCTGATTATTTGAAACTTAGTTTTCCTGAGGGTTTTAAGTGG
GAACGCGTTATGAATTTCGAGGATGGAGGAGTAGTGACAGTAACTCAAG
ATTCCAGTCTTCAAGACGGAGAGTTTATCTACAAGGTGAAATTACGTGG
CACGAACTTTCCATCCGACGGGCCAGTGATGCAGAAGAAAACCATGGGT
TGGGAGGCATCTTCTGAGCGTATGTACCCGGAAGACGGGGCGTTGAAAG
GCGAAATCAAGCAGCGCCTGAAGTTGAAAGACGGGGGTCACTACGATGC
GGAAGTAAAAACTACATATAAGGCCAAGAAGCCCGTCCAGCTTCCGGGC
GCGTACAACGTCAACATCAAATTGGACATTACTTCCCACAACGAGGATT
ACACTATTGTTGAACAATATGAGCGTGCGGAGGGACGCCATTCAACCGG
GGGGATGGATGAGCTTTACAAG
(mCherry DNA sequence codon-optimized for
H. sapiens)
SEQ ID NO: 346
ATGGTCAGTAAGGGGGAAGAGGACAACATGGCGATAATCAAGGAATTTA
TGAGATTTAAGGTCCATATGGAAGGCTCTGTCAACGGTCATGAGTTCGA
AATTGAGGGGGAGGGGGAAGGCCGCCCATATGAGGGGACTCAAACAGCC
AAACTTAAGGTCACAAAAGGAGGTCCTCTGCCCTTCGCGTGGGACATAC
TCAGCCCACAATTCATGTATGGAAGTAAGGCATATGTTAAACACCCGGC
GGACATACCCGACTACCTCAAACTGTCATTTCCTGAGGGTTTTAAGTGG
GAGAGGGTTATGAATTTCGAGGACGGTGGAGTAGTAACAGTGACACAAG
ACTCTTCTCTCCAGGATGGAGAATTTATATACAAAGTGAAACTGCGGGG
CACTAACTTTCCGTCAGACGGCCCAGTAATGCAAAAGAAAACTATGGGG
TGGGAGGCTTCCAGCGAGCGCATGTATCCCGAGGATGGGGCCCTTAAGG
GAGAAATAAAACAACGCTTGAAGCTCAAAGACGGAGGGCACTATGATGC
GGAGGTCAAAACGACCTACAAAGCAAAAAAGCCAGTACAGCTTCCGGGA
GCTTATAACGTGAATATAAAGCTCGATATAACCTCACACAACGAGGACT
ACACGATTGTAGAACAATACGAAAGAGCCGAGGGGAGACATAGTACGGG
GGGCATGGACGAACTTTACAAG

### Example 10

### Sequences of Chimeric Proteins

Table V.1 provides chimera fusion proteins between Cas9 amino acid sequences, linker amino acid sequences (underlined), and eGFP amino acid sequences (bolded, italicized).

TABLE V.1
Cas9-Linker-eGFP Chimeras

SEQ ID NO: 38
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
GSAGSAAGSGEF
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 39
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
GGGGSGGGGSGGGGSGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 40
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
AEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 41
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
AEAAAKEAAAKEAAAKEAAAKALEAEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 42
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
LEAEAAAKEAAAKEAAAKEAAAKALEAEAAAKEAAAKEAAAKEAAAKALE
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 43
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 44
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 45
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 46
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 47
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 48
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 49
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 50
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 51
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 52
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 53
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 54
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 55
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 56
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 57
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 58
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPFMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 59
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 60
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 61
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 62
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 63
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 64
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 65
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 66
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 67
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 68
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 69
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 70
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 71
MDKKYSIGLDIGINSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLEGNLIALSLGLIPNEKSNEDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNEDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKEDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGESKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
GGGGSEAAAKGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK

Table V.2 provides chimera fusion proteins between Cas9 amino acid sequences, linker amino acid sequences (underlined), and mCherry amino acid sequences (bolded, italicized).

TABLE V.2
Cas9-Linker-mCherryChimeras

SEQ ID NO: 72
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
GSAGSAAGSGEF
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 73
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
GGGGSGGGGSGGGGSGGGGS
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 74
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
AEAAAKEAAAKEAAAKEAAAKA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 75
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
AEAAAKEAAAKEAAAKEAAAKALEAEAAAKEAAAKEAAAKEAAAKA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 76
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
LEAEAAAKEAAAKEAAAKEAAAKALEAEAAAKEAAAKEAAAKEAAAKALE
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 77
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 78
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 79
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 80
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 81
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 82
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 83
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 84
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 85
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
APAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 86
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 87
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 88
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 89
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 90
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
APAPAPAPAPAPAPAPAPAPAPAPAPAPA
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 91
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 92
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 93
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 94
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 95
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 96
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 97
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 98
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 99
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 100
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
XPXPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 101
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 102
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 103
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 104
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
XPXPXPXPXPXPXPXPXPXPXPXPXPXP
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 105
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
GGGGSEAAAKGGGGS
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK

Table V.3 provides chimera fusion proteins between AsCas12a amino acid sequences, linker amino acid sequences (underlined), and eGFP amino acid sequences (bolded, italicized).

TABLE V.3
AsCas12a-Linker-eGFP Chimeras

SEQ ID NO: 106
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
GSAGSAAGSGEFMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 107
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
GGGGSGGGGSGGGGSGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 108
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
AEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 109
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
AEAAAKEAAAKEAAAKEAAAKALEAEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 110
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
LEAEAAAKEAAAKEAAAKEAAAKALEAEAAAKEAAAKEAAAKEAAAKALE
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 111
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 112
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 113
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 114
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 115
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 116
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAMVSKGEELFTGVVPILVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRAEVKFEGDTLVNRIELKGIDFKEDGNILG
HKLEYNYNSHNVYIMADKQKNGIKVNFKIRHNIEDGSVQLADHYQQNTPIGDGPVLLPDNHYLSTQSALSKDPNEK
RDHMVLLEFVTAAGITLGMDELYK
SEQ ID NO: 117
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 118
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 119
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 120
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 121
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 122
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 123
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 124
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
APAPAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 125
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 126
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 127
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 128
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 129
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPMVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 130
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 131
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 132
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 133
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 134
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 135
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 136
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 137
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 138
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
XPXPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 139
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKFTTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKFFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGFMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRN
GGGGSEAAAKGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK

Table V.4 provides chimera fusion proteins between AsCas12a amino acid sequences, linker amino acid sequences (underlined), and mCherry amino acid sequences (bolded, italicized).

TABLE V.4
AsCas12a-Linker-mCherry Chimeras

SEQ ID NO: 140
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
GSAGSAAGSGEF
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 141
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
GGGGSGGGGSGGGGSGGGGS
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 142
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
AEAAAKEAAAKEAAAKEAAAKA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 143
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
AEAAAKEAAAKEAAAKEAAAKALEAEAAAKEAAAKEAAAKEAAAKA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 144
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
LEAEAAAKEAAAKEAAAKEAAAKALEAEAAAKEAAAKEAAAKEAAAKALE
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 145
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 146
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 147
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDR RFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 148
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 149
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 150
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 151
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 152
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 153
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 154
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 155
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 156
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 157
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 158
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
APAPAPAPAPAPAPAPAPAPAPAPAPAPA
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 159
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 160
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 161
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 162
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 163
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 164
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 165
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 166
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 167
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 168
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 169
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSFDKETTYFSGEYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 170
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 171
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 172
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
XPXPXPXPXPXPXPXPXPXPXPXPXPXP
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 173
MTQFEGFTNLYQVSKTLRFELIPQGKILKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELENGKVLKQLGTVITTEH
ENALLRSEDKETTYFSGEYENRKNVESAEDISTAIPHRIVQDNFPKEKENCHIFTRLITAVPSLREHFENVKKAIGI
FVSTSIEEVESFPFYNQLLTQTQIDLYNQLLGGISREAGTEKIKGLNEVLNLAIQKNDETAHIIASLPHRFIPLEKQ
ILSDRNTLSFILEEEKSDEEVIQSECKYKILLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLIGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDETRDELSKYTKITSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKENQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNEGEKS
KRTGIAEKAVYQQFEKMLIDKLNCLVLKDYPAEKVGGVLNPYQLTDQFTSFAKMGTQSGELFYVPAPYTSKIDPLIG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVERDGSNILPKLLENDDSHAIDTMVALIRSVLQMRNSNAAT
GEDYINSPVRDLNGVCEDSREQNPEWPMDADANGAYHIALKGQLLLNHLKESKDLKLQNGISNQDWLAYIQELRN
GGGGSEAAAKGGGGS
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK

Table V.5 provides chimera fusion proteins between LbCas12a amino acid sequences, linker amino acid sequences (underlined), and eGFP amino acid sequences (bolded, italicized).

TABLE V.5
LbCas12a-Linker-eGFP Chimeras

SEQ ID NO: 174
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GSAGSAAGSGEF
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 175
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSGGGGSGGGGSGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 176
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 177
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKALEAEAAAKEAAAKEAAAKEAAAKA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 178
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
LEAEAAAKEAAAKEAAAKEAAAKALEAEAAAKEAAAKEAAAKEAAAKALE
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 179
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 180
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 181
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 182
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 183
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 184
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 185
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 186
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 187
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 188
MTQFEGFTNLYQVSKTLRFELIPQGKTLKHIQEQGFIEEDKARNDHYKELKPIIDRIYKTYADQCLQLVQLDWENLS
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 189
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 190
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 191
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 192
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAPA
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 193
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 194
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 195
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 196
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 197
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 198
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 199
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 200
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 201
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 202
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 203
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 204
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 205
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 206
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPXP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 207
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSEAAAKGGGGS
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK

Table V.6 provides chimera fusion proteins between LbCas12a amino acid sequences, linker amino acid sequences (underlined), and mCherry amino acid sequences (bolded, italicized).

TABLE V.6
LbCas12a-Linker-mCherry Chimeras

SEQ ID NO: 208
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GSAGSAAGSGEFMVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 209
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSGGGGSGGGGSGGGGSMVSKGEEDNMAIIKEFMRFKVHMEGSVNGHEFEIEGEGEGRPYEGTQTAKLKVTKG
GPLPFAWDILSPQFMYGSKAYVKHPADIPDYLKLSFPEGFKWERVMNFEDGGVVTVTQDSSLQDGEFIYKVKLRGT
NFPSDGPVMQKKTMGWEASSERMYPEDGALKGEIKQRLKLKDGGHYDAEVKTTYKAKKPVQLPGAYNVNIKLDITS
HNEDYTIVEQYERAEGRHSTGGMDELYK
SEQ ID NO: 210
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKAMVSKGEEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMNFEDGGVVTVTQDSSLQDGEFIYKVKLR
GTNFPSDGPVMQKKTMGWEASSERMYPEDGALKGEIKQRLKLKDGGHYDAEVKTTYKAKKPVQLPGAYNVNIKLDI
TSHNEDYTIVEQYERAEGRHSTGGMDELYK
SEQ ID NO: 211
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKALEAEAAAKEAAAKEAAAKEAAAKAMVSKGEEDNMAIIKEFMRFKVHMEGSVNGH
EFEIEGEGEGRPYEGTQTAKLKVTKGGPLPFAWDILSPQFMYGSKAYVKHPADIPDYLKLSFPEGFKWERVMNFED
GGVVTVTQDSSLQDGEFIYKVKLRGTNFPSDGPVMQKKTMGWEASSERMYPEDGALKGEIKQRLKLKDGGHYDAEV
KTTYKAKKPVQLPGAYNVNIKLDITSHNEDYTIVEQYERAEGRHSTGGMDELYK
SEQ ID NO: 212
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
LEAEAAAKEAAAKEAAAKEAAAKALEAEAAAKEAAAKEAAAKEAAAKALEMVSKGEEDNMAIIKEFMRFKVHMEGS
VNGHEFEIEGEGEGRPYEGTQTAKLKVTKGGPLPFAWDILSPQFMYGSKAYVKHPADIPDYLKLSFPEGFKWERVM
NFEDGGVVTVTQDSSLQDGEFIYKVKLRGTNFPSDGPVMQKKTMGWEASSERMYPEDGALKGEIKQRLKLKDGGHY
DAEVKTTYKAKKPVQLPGAYNVNIKLDITSHNEDYTIVEQYERAEGRHSTGGMDELYK
SEQ ID NO: 213
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAMVSKGEEDNMAIIKEFMRFKVHMEGSVNGHEFEIEGEGEGRPYEGTQTAKLKVTKGGPLPFAWDILSPQFMYG
SKAYVKHPADIPDYLKLSFPEGFKWERVMNFEDGGVVTVTQDSSLQDGEFIYKVKLRGTNFPSDGPVMQKKTMGWE
ASSERMYPEDGALKGEIKQRLKLKDGGHYDAEVKTTYKAKKPVQLPGAYNVNIKLDITSHNEDYTIVEQYERAEGR
HSTGGMDELYK
SEQ ID NO: 214
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAMVSKGEEDNMAIIKEFMRFKVHMEGSVNGHEFEIEGEGEGRPYEGTQTAKLKVTKGGPLPFAWDILSPQFM
YGSKAYVKHPADIPDYLKLSFPEGFKWERVMNFEDGGVVTVTQDSSLQDGEFIYKVKLRGTNFPSDGPVMQKKTMG
WEASSERMYPEDGALKGEIKQRLKLKDGGHYDAEVKTTYKAKKPVQLPGAYNVNIKLDITSHNEDYTIVEQYERAE
GRHSTGGMDELYK
SEQ ID NO: 215
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAMVSKGEEDNMAIIKEFMRFKVHMEGSVNGHEFEIEGEGEGRPYEGTQTAKLKVTKGGPLPFAWDILSPQ
FMYGSKAYVKHPADIPDYLKLSFPEGFKWERVMNFEDGGVVTVTQDSSLQDGEFIYKVKLRGTNFPSDGPVMQKKT
MGWEASSERMYPEDGALKGEIKQRLKLKDGGHYDAEVKTTYKAKKPVQLPGAYNVNIKLDITSHNEDYTIVEQYER
AEGRHSTGGMDELYK
SEQ ID NO: 216
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAMVSKGEEDNMAIIKEFMRFKVHMEGSVNGHEFEIEGEGEGRPYEGTQTAKLKVTKGGPLPFAWDILS
PQFMYGSKAYVKHPADIPDYLKLSFPEGFKWERVMNFEDGGVVTVTQDSSLQDGEFIYKVKLRGTNFPSDGPVMQK
KTMGWEASSERMYPEDGALKGEIKQRLKLKDGGHYDAEVKTTYKAKKPVQLPGAYNVNIKLDITSHNEDYTIVEQY
ERAEGRHSTGGMDELYK
SEQ ID NO: 217
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAMVSKGEEDNMAIIKEFMRFKVHMEGSVNGHEFEIEGEGEGRPYEGTQTAKLKVTKGGPLPFAWDI
LSPQFMYGSKAYVKHPADIPDYLKLSFPEGFKWERVMNFEDGGVVTVTQDSSLQDGEFIYKVKLRGTNFPSDGPVM
QKKTMGWEASSERMYPEDGALKGEIKQRLKLKDGGHYDAEVKTTYKAKKPVQLPGAYNVNIKLDITSHNEDYTIVE
QYERAEGRHSTGGMDELYK
SEQ ID NO: 218
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAMVSKGEEDNMAIIKEFMRFKVHMEGSVNGHEFEIEGEGEGRPYEGTQTAKLKVTKGGPLPFAW
DILSPQFMYGSKAYVKHPADIPDYLKLSFPEGFKWERVMNFEDGGVVTVTQDSSLQDGEFIYKVKLRGTNFPSDGP
VMQKKTMGWEASSERMYPEDGALKGEIKQRLKLKDGGHYDAEVKTTYKAKKPVQLPGAYNVNIKLDITSHNEDYTI
VEQYERAEGRHSTGGMDELYK
SEQ ID NO: 219
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAMVSKGEEDNMAIIKEFMRFKVHMEGSVNGHEFEIEGEGEGRPYEGTQTAKLKVTKGGPLPF
AWDILSPQFMYGSKAYVKHPADIPDYLKLSFPEGFKWERVMNFEDGGVVTVTQDSSLQDGEFIYKVKLRGTNFPSD
GPVMQKKTMGWEASSERMYPEDGALKGEIKQRLKLKDGGHYDAEVKTTYKAKKPVQLPGAYNVNIKLDITSHNEDY
TIVEQYERAEGRHSTGGMDELYK
SEQ ID NO: 220
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAMVSKGEEDNMAIIKEFMRFKVHMEGSVNGHEFEIEGEGEGRPYEGTQTAKLKVTKGGPL
PFAWDILSPQFMYGSKAYVKHPADIPDYLKLSFPEGFKWERVMNFEDGGVVTVTQDSSLQDGEFIYKVKLRGTNFP
SDGPVMQKKTMGWEASSERMYPEDGALKGEIKQRLKLKDGGHYDAEVKTTYKAKKPVQLPGAYNVNIKLDITSHNE
DYTIVEQYERAEGRHSTGGMDELYK
SEQ ID NO: 221
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAMVSKGEEDNMAIIKEFMRFKVHMEGSVNGHEFEIEGEGEGRPYEGTQTAKLKVTKGG
PLPFAWDILSPQFMYGSKAYVKHPADIPDYLKLSFPEGFKWERVMNFEDGGVVTVTQDSSLQDGEFIYKVKLRGTN
FPSDGPVMQKKTMGWEASSERMYPEDGALKGEIKQRLKLKDGGHYDAEVKTTYKAKKPVQLPGAYNVNIKLDITSH
NEDYTIVEQYERAEGRHSTGGMDELYK
SEQ ID NO: 222
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAMVSKGEEDNMAIIKEFMRFKVHMEGSVNGHEFEIEGEGEGRPYEGTQTAKLKVTK
GGPLPFAWDILSPQFMYGSKAYVKHPADIPDYLKLSFPEGFKWERVMNFEDGGVVTVTQDSSLQDGEFIYKVKLRG
TNFPSDGPVMQKKTMGWEASSERMYPEDGALKGEIKQRLKLKDGGHYDAEVKTTYKAKKPVQLPGAYNVNIKLDIT
SHNEDYTIVEQYERAEGRHSTGGMDELYK
SEQ ID NO: 223
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAMVSKGEEDNMAIIKEFMRFKVHMEGSVNGHEFEIEGEGEGRPYEGTQTAKLKV
TKGGPLPFAWDILSPQFMYGSKAYVKHPADIPDYLKLSFPEGFKWERVMNFEDGGVVTVTQDSSLQDGEFIYKVKL
RGTNFPSDGPVMQKKTMGWEASSERMYPEDGALKGEIKQRLKLKDGGHYDAEVKTTYKAKKPVQLPGAYNVNIKLD
ITSHNEDYTIVEQYERAEGRHSTGGMDELYK
SEQ ID NO: 224
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAMVSKGEEDNMAIIKEFMRFKVHMEGSVNGHEFEIEGEGEGRPYEGTQTAKL
KVTKGGPLPFAWDILSPQFMYGSKAYVKHPADIPDYLKLSFPEGFKWERVMNFEDGGVVTVTQDSSLQDGEFIYKV
KLRGTNFPSDGPVMQKKTMGWEASSERMYPEDGALKGEIKQRLKLKDGGHYDAEVKTTYKAKKPVQLPGAYNVNIK
LDITSHNEDYTIVEQYERAEGRHSTGGMDELYK
SEQ ID NO: 225
MSKLEKFTNCYSLSKTLRFKAIPVGKTQENIDNKRLLVEDEKRAEDYKGVKKLLDRYYLSFINDVLHSIKLKNLNNY
ISLERKKIRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLIQ
EGIDVYNAIIGGFVTESGEKIKGLNEYINLYNQKTKQKLPKFKPLYKQVLSDRESLSFYGEGYISDEEVLEVERNIL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAMVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKWERVMNFEDGGVVTVTQDSSLQDGEFIY
KVKLRGTNFPSDGPVMQKKTMGWEASSERMYPEDGALKGEIKQRLKLKDGGHYDAEVKTTYKAKKPVQLPGAYNVN
IKLDITSHNEDYTIVEQYERAEGRHSTGGMDELYK
SEQ ID NO: 226
MSKLEKFTNCYSLSKTLRFKAIPVGKTQENIDNKRLLVEDEKRAEDYKGVKKLLDRYYLSFINDVLHSIKLKNLNNY
ISLERKKIRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLIQ
EGIDVYNAIIGGFVTESGEKIKGLNEYINLYNQKTKQKLPKFKPLYKQVLSDRESLSFYGEGYISDEEVLEVERNIL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAPAMVSKGEEDNMAIIKEFMRFKVHMEGSVNGHEFEIEGEGEGRPYEGTQ
TAKLKVTKGGPLPFAWDILSPQFMYGSKAYVKHPADIPDYLKLSFPEGFKWERVMNFEDGGVVTVTQDSSLQDGEF
IYKVKLRGTNFPSDGPVMQKKTMGWEASSERMYPEDGALKGEIKQRLKLKDGGHYDAEVKTTYKAKKPVQLPGAYN
VNIKLDITSHNEDYTIVEQYERAEGRHSTGGMDELYK
SEQ ID NO: 227
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPMVSKGEEDNMAIIKEFMRFKVHMEGSVNGHEFEIEGEGEGRPYEGTQTAKLKVTKGGPLPFAWDILSPQFMYGS
KAYVKHPADIPDYLKLSFPEGFKWERVMNFEDGGVVTVTQDSSLQDGEFIYKVKLRGTNFPSDGPVMQKKTMGWEA
SSERMYPEDGALKGEIKQRLKLKDGGHYDAEVKTTYKAKKPVQLPGAYNVNIKLDITSHNEDYTIVEQYERAEGRH
STGGMDELYK
SEQ ID NO: 228
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPMVSKGEEDNMAIIKEFMRFKVHMEGSVNGHEFEIEGEGEGRPYEGTQTAKLKVTKGGPLPFAWDILSPQFMY
GSKAYVKHPADIPDYLKLSFPEGFKWERVMNFEDGGVVTVTQDSSLQDGEFIYKVKLRGTNFPSDGPVMQKKTMGW
EASSERMYPEDGALKGEIKQRLKLKDGGHYDAEVKTTYKAKKPVQLPGAYNVNIKLDITSHNEDYTIVEQYERAEG
RHSTGGMDELYK
SEQ ID NO: 229
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPMVSKGEEDNMAIIKEFMRFKVHMEGSVNGHEFEIEGEGEGRPYEGTQTAKLKVTKGGPLPFAWDILSPQF
MYGSKAYVKHPADIPDYLKLSFPEGFKWERVMNFEDGGVVTVTQDSSLQDGEFIYKVKLRGTNFPSDGPVMQKKTM
GWEASSERMYPEDGALKGEIKQRLKLKDGGHYDAEVKTTYKAKKPVQLPGAYNVNIKLDITSHNEDYTIVEQYERA
EGRHSTGGMDELYK
SEQ ID NO: 230
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPMVSKGEEDNMAIIKEFMRFKVHMEGSVNGHEFEIEGEGEGRPYEGTQTAKLKVTKGGPLPFAWDILSP
QFMYGSKAYVKHPADIPDYLKLSFPEGFKWERVMNFEDGGVVTVTQDSSLQDGEFIYKVKLRGTNFPSDGPVMQKK
TMGWEASSERMYPEDGALKGEIKQRLKLKDGGHYDAEVKTTYKAKKPVQLPGAYNVNIKLDITSHNEDYTIVEQYE
RAEGRHSTGGMDELYK
SEQ ID NO: 231
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPMVSKGEEDNMAIIKEFMRFKVHMEGSVNGHEFEIEGEGEGRPYEGTQTAKLKVTKGGPLPFAWDIL
SPQFMYGSKAYVKHPADIPDYLKLSFPEGFKWERVMNFEDGGVVTVTQDSSLQDGEFIYKVKLRGTNFPSDGPVMQ
KKTMGWEASSERMYPEDGALKGEIKQRLKLKDGGHYDAEVKTTYKAKKPVQLPGAYNVNIKLDITSHNEDYTIVEQ
YERAEGRHSTGGMDELYK
SEQ ID NO: 232
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPMVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAKKPVQLPGAYNVNIKLDITSHNEDYTIV
EQYERAEGRHSTGGMDELYK
SEQ ID NO: 233
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPMVSKGEEDNMAIIKEFMRFKVHMEGSVNGHEFEIEGEGEGRPYEGTQTAKLKVTKGGPLPFA
WDILSPQFMYGSKAYVKHPADIPDYLKLSFPEGFKWERVMNFEDGGVVTVTQDSSLQDGEFIYKVKLRGTNFPSDG
PVMQKKTMGWEASSERMYPEDGALKGEIKQRLKLKDGGHYDAEVKTTYKAKKPVQLPGAYNVNIKLDITSHNEDYT
IVEQYERAEGRHSTGGMDELYK
SEQ ID NO: 234
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPMVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLQDGEFIYKVKLRGTNFPS
DGPVMQKKTMGWEASSERMYPEDGALKGEIKQRLKLKDGGHYDAEVKTTYKAKKPVQLPGAYNVNIKLDITSHNED
YTIVEQYERAEGRHSTGGMDELYK
SEQ ID NO: 235
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPMVSKGEEDNMAIIKEFMRFKVHMEGSVNGHEFEIEGEGEGRPYEGTQTAKLKVTKGGP
LPFAWDILSPQFMYGSKAYVKHPADIPDYLKLSFPEGFKWERVMNFEDGGVVTVTQDSSLQDGEFIYKVKLRGTNF
PSDGPVMQKKTMGWEASSERMYPEDGALKGEIKQRLKLKDGGHYDAEVKTTYKAKKPVQLPGAYNVNIKLDITSHN
EDYTIVEQYERAEGRHSTGGMDELYK
SEQ ID NO: 236
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPMVSKGEEDNMAIIKEFMRFKVHMEGSVNGHEFEIEGEGEGRPYEGTQTAKLKVTKG
GPLPFAWDILSPQFMYGSKAYVKHPADIPDYLKLSFPEGFKWERVMNFEDGGVVTVTQDSSLQDGEFIYKVKLRGT
NFPSDGPVMQKKTMGWEASSERMYPEDGALKGEIKQRLKLKDGGHYDAEVKTTYKAKKPVQLPGAYNVNIKLDITS
HNEDYTIVEQYERAEGRHSTGGMDELYK
SEQ ID NO: 237
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPMVSKGEEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMNFEDGGVVTVTQDSSLQDGEFIYKVKLR
GTNFPSDGPVMQKKTMGWEASSERMYPEDGALKGEIKQRLKLKDGGHYDAEVKTTYKAKKPVQLPGAYNVNIKLDI
TSHNEDYTIVEQYERAEGRHSTGGMDELYK
SEQ ID NO: 238
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPMVSKGEEDNMAIIKEFMRFKVHMEGSVNGHEFEIEGEGEGRPYEGTQTAKLK
VTKGGPLPFAWDILSPQFMYGSKAYVKHPADIPDYLKLSFPEGFKWERVMNFEDGGVVTVTQDSSLQDGEFIYKVK
LRGTNFPSDGPVMQKKTMGWEASSERMYPEDGALKGEIKQRLKLKDGGHYDAEVKTTYKAKKPVQLPGAYNVNIKL
DITSHNEDYTIVEQYERAEGRHSTGGMDELYK
SEQ ID NO: 239
MSKLEKFTNCYSLSKTLRFKAIPVGKTQENIDNKRLLVEDEKRAEDYKGVKKLLDRYYLSFINDVLHSIKLKNLNNY
ISLERKKIRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLIQ
EGIDVYNAIIGGFVTESGEKIKGLNEYINLYNQKTKQKLPKFKPLYKQVLSDRESLSFYGEGYISDEEVLEVERNIL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPMVSKGEEDNMAIIKEFMRFKVHMEGSVNGHEFEIEGEGEGRPYEGTQTAK
LKVTKGGPLPFAWDILSPQFMYGSKAYVKHPADIPDYLKLSFPEGFKWERVMNFEDGGVVTVTQDSSLQDGEFIYK
VKLRGTNFPSDGPVMQKKTMGWEASSERMYPEDGALKGEIKQRLKLKDGGHYDAEVKTTYKAKKPVQLPGAYNVNI
KLDITSHNEDYTIVEQYERAEGRHSTGGMDELYK
SEQ ID NO: 240
MSKLEKFTNCYSLSKTLRFKAIPVGKTQENIDNKRLLVEDEKRAEDYKGVKKLLDRYYLSFINDVLHSIKLKNLNNY
ISLERKKIRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLIQ
EGIDVYNAIIGGFVTESGEKIKGLNEYINLYNQKTKQKLPKFKPLYKQVLSDRESLSFYGEGYISDEEVLEVERNIL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPXPMVSKGEEDNMAIIKEFMRFKVHMEGSVNGHEFEIEGEGEGRPYEGTQT
AKLKVTKGGPLPFAWDILSPQFMYGSKAYVKHPADIPDYLKLSFPEGFKWERVMNFEDGGVVTVTQDSSLQDGEFI
YKVKLRGTNFPSDGPVMQKKTMGWEASSERMYPEDGALKGEIKQRLKLKDGGHYDAEVKTTYKAKKPVQLPGAYNV
NIKLDITSHNEDYTIVEQYERAEGRHSTGGMDELYK
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSEAAAKGGGGSMVSKGEEDNMAIIKEFMRFKVHMEGSVNGHEFEIEGEGEGRPYEGTQTAKLKVTKGGPLPF
AWDILSPQFMYGSKAYVKHPADIPDYLKLSFPEGFKWERVMNFEDGGVVTVTQDSSLQDGEFIYKVKLRGTNFPSD
GPVMQKKTMGWEASSERMYPEDGALKGEIKQRLKLKDGGHYDAEVKTTYKAKKPVQLPGAYNVNIKLDITSHNEDY
TIVEQYERAEGRHSTGGMDELYK

Table V.7 provides chimera fusion proteins between LbCas12a variant amino acid sequences (E795L), linker amino acid sequences (underlined), and eGFP amino acid sequences (bolded, italicized).

TABLE V.7
LbCas12a(E795L)-Linker-eGFP Chimeras

SEQ ID NO: 242
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GSAGSAAGSGEFMVSKGEELFTGVVPILVELDGDVNGHKFSVSGEGEGDATYGKLTLKFICTTGKLPVPWPTLVTTV
TYGVQCFSRYPDHMKQHDFFKSAMPEGYVQERTIFFKDDGNYKTRAEVKFEGDTLVNRIELKGIDFKEDGNILGHKL
EYNYNSHNVYIMADKQKNGIKVNFKIRHNIEDGSVQLADHYQQNTPIGDGPVLLPDNHYLSTQSALSKDPNEKRDHM
VLLEFVTAAGITLGMDELYK
SEQ ID NO: 243
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSGGGGSGGGGSGGGGSMVSKGEELFTGVVPILVELDGDVNGHKFSVSGEGEGDATYGKLTLKFICTTGKLPVP
WPTLVTTVTYGVQCFSRYPDHMKQHDFFKSAMPEGYVQERTIFFKDDGNYKTRAEVKFEGDTLVNRIELKGIDFKED
GNILGHKLEYNYNSHNVYIMADKQKNGIKVNFKIRHNIEDGSVQLADHYQQNTPIGDGPVLLPDNHYLSTQSALSKD
PNEKRDHMVLLEFVTAAGITLGMDELYK
SEQ ID NO: 244
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKAMVSKGEELFTGVVPILVELDGDVNGHKFSVSGEGEGDATYGKLTLKFICTTGKLP
VPWPTLVTTVTYGVQCFSRYPDHMKQHDFFKSAMPEGYVQERTIFFKDDGNYKTRAEVKFEGDTLVNRIELKGIDFK
EDGNILGHKLEYNYNSHNVYIMADKQKNGIKVNFKIRHNIEDGSVQLADHYQQNTPIGDGPVLLPDNHYLSTQSALS
KDPNEKRDHMVLLEFVTAAGITLGMDELYK
SEQ ID NO: 245
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKALEAEAAAKEAAAKEAAAKEAAAKAMVSKGEELFTGVVPILVELDGDVNGHKFSVS
GEGEGDATYGKLTLKFICTTGKLPVPWPTLVTTVTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQKNGIKVNFKIRHNIEDGSVQLADHYQQ
NTPIGDGPVLLPDNHYLSTQSALSKDPNEKRDHMVLLEFVTAAGITLGMDELYK
SEQ ID NO: 246
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
LEAEAAAKEAAAKEAAAKEAAAKALEAEAAAKEAAAKEAAAKEAAAKALEMVSKGEELFTGVVPILVELDGDVNGHK
FSVSGEGEGDATYGKLTLKFICTTGKLPVPWPTLVTTVTYGVQCFSRYPDHMKQHDFFKSAMPEGYVQERTIFFKDD
GNYKTRAEVKFEGDTLVNRIELKGIDFKEDGNILGHKLEYNYNSHNVYIMADKQKNGIKVNFKIRHNIEDGSVQLAD
HYQQNTPIGDGPVLLPDNHYLSTQSALSKDPNEKRDHMVLLEFVTAAGITLGMDELYK
SEQ ID NO: 247
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAMVSKGEELFTGVVPILVELDGDVNGHKFSVSGEGEGDATYGKLTLKFICTTGKLPVPWPTLVTTVTYGVQCFSR
YPDHMKQHDFFKSAMPEGYVQERTIFFKDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHYLSTQSALSKDPNEKRDHMVLLEFVTAA
GITLGMDELYK
SEQ ID NO: 248
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAMVSKGEELFTGVVPILVELDGDVNGHKFSVSGEGEGDATYGKLTLKFICTTGKLPVPWPTLVTTVTYGVQCF
SRYPDHMKQHDFFKSAMPEGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSALSKDPNEKRDHMVLLEFVT
AAGITLGMDELYK
SEQ ID NO: 249
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAMVSKGEELFTGVVPILVELDGDVNGHKFSVSGEGEGDATYGKLTLKFICTTGKLPVPWPTLVTTVTYGVQ
CFSRYPDHMKQHDFFKSAMPEGYVQERTIFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLLPDNHYLSTQSALSKDPNEKRDHMVLLEF
VTAAGITLGMDELYK
SEQ ID NO: 250
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAMVSKGEELFTGVVPILVELDGDVNGHKFSVSGEGEGDATYGKLTLKFICTTGKLPVPWPTLVTTVTYG
VQCFSRYPDHMKQHDFFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSALSKDPNEKRDHMVLL
EFVTAAGITLGMDELYK
SEQ ID NO: 251
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAMVSKGEELFTGVVPILVELDGDVNGHKFSVSGEGEGDATYGKLTLKFICTTGKLPVPWPTLVTTVT
YGVQCFSRYPDHMKQHDFFKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHYLSTQSALSKDPNEKRDHMV
LLEFVTAAGITLGMDELYK
SEQ ID NO: 252
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAMVSKGEELFTGVVPILVELDGDVNGHKFSVSGEGEGDATYGKLTLKFICTTGKLPVPWPTLVTT
VTYGVQCFSRYPDHMKQHDFFKSAMPEGYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGDGPVLLPDNHYLSTQSALSKDPNEKRDH
MVLLEFVTAAGITLGMDELYK
SEQ ID NO: 253
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAMVSKGEELFTGVVPILVELDGDVNGHKFSVSGEGEGDATYGKLTLKFICTTGKLPVPWPTLV
TTVTYGVQCFSRYPDHMKQHDFFKSAMPEGYVQERTIFFKDDGNYKTRAEVKFEGDTLVNRIELKGIDFKEDGNILG
HKLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK
SEQ ID NO: 254
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAMVSKGEELFTGVVPILVELDGDVNGHKFSVSGEGEGDATYGKLTLKFICTTGKLPVPWPT
LVTTVTYGVQCFSRYPDHMKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLLPDNHYLSTQSALSKDPNE
KRDHMVLLEFVTAAGITLGMDELYK
SEQ ID NO: 255
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAMVSKGEELFTGVVPILVELDGDVNGHKFSVSGEGEGDATYGKLTLKFICTTGKLPVPW
PTLVTTVTYGVQCFSRYPDHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPVLLPDNHYLSTQSALSKDP
NEKRDHMVLLEFVTAAGITLGMDELYK
SEQ ID NO: 256
MTQFEGFTNLYQVSKTLRFELIPQGKTLKHIQEQGFIEEDKARNDHYKELKPIIDRIYKTYADQCLQLVQLDWENLS
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAMVSKGEELFTGVVPILVELDGDVNGHKFSVSGEGEGDATYGKLTLKFICTTGKLPV
PWPTLVTTVTYGVQCFSRYPDHMKQHDFFKSAMPEGYVQERTIFFKDDGNYKTRAEVKFEGDTLVNRIELKGIDFKE
DGNILGHKLEYNYNSHNVYIMADKQKNGIKVNFKIRHNIEDGSVQLADHYQQNTPIGDGPVLLPDNHYLSTQSALSK
DPNEKRDHMVLLEFVTAAGITLGMDELYK
SEQ ID NO: 257
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAMVSKGEELFTGVVPILVELDGDVNGHKFSVSGEGEGDATYGKLTLKFICTTGKL
PVPWPTLVTTVTYGVQCFSRYPDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTPIGDGPVLLPDNHYLSTQSAL
SKDPNEKRDHMVLLEFVTAAGITLGMDELYK
SEQ ID NO: 258
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAMVSKGEELFTGVVPILVELDGDVNGHKFSVSGEGEGDATYGKLTLKFICTTG
KLPVPWPTLVTTVTYGVQCFSRYPDHMKQHDFFKSAMPEGYVQERTIFFKDDGNYKTRAEVKFEGDTLVNRIELKGI
DFKEDGNILGHKLEYNYNSHNVYIMADKQKNGIKVNFKIRHNIEDGSVQLADHYQQNTPIGDGPVLLPDNHYLSTQS
ALSKDPNEKRDHMVLLEFVTAAGITLGMDELYK
SEQ ID NO: 259
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAMVSKGEELFTGVVPILVELDGDVNGHKFSVSGEGEGDATYGKLTLKFICT
TGKLPVPWPTLVTTVTYGVQCFSRYPDHMKQHDFFKSAMPEGYVQERTIFFKDDGNYKTRAEVKFEGDTLVNRIELK
GIDFKEDGNILGHKLEYNYNSHNVYIMADKQKNGIKVNFKIRHNIEDGSVQLADHYQQNTPIGDGPVLLPDNHYLST
QSALSKDPNEKRDHMVLLEFVTAAGITLGMDELYK
SEQ ID NO: 260
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAPAMVSKGEELFTGVVPILVELDGDVNGHKFSVSGEGEGDATYGKLTLKFI
CTTGKLPVPWPTLVTTVTYGVQCFSRYPDHMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLADHYQQNTPIGDGPVLLPDNHYL
STQSALSKDPNEKRDHMVLLEFVTAAGITLGMDELYK
SEQ ID NO: 261
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPMVSKGEELFTGVVPILVELDGDVNGHKFSVSGEGEGDATYGKLTLKFICTTGKLPVPWPTLVTTVTYGVQCFSRY
PDHMKQHDFFKSAMPEGYVQERTIFFKDDGNYKTRAEVKFEGDTLVNRIELKGIDFKEDGNILGHKLEYNYNSHNVY
IMADKQKNGIKVNFKIRHNIEDGSVQLADHYQQNTPIGDGPVLLPDNHYLSTQSALSKDPNEKRDHMVLLEFVTAAG
ITLGMDELYK
SEQ ID NO: 262
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPMVSKGEELFTGVVPILVELDGDVNGHKFSVSGEGEGDATYGKLTLKFICTTGKLPVPWPTLVTTVTYGVQCFS
RYPDHMKQHDFFKSAMPEGYVQERTIFFKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNHYLSTQSALSKDPNEKRDHMVLLEFVTA
AGITLGMDELYK
SEQ ID NO: 263
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXMVSKGEELFTGVVPILVELDGDVNGHKFSVSGEGEGDATYGKLTLKFICTTGKLPVPWPTLVTTVTYGVQCF
SRYPDHMKQHDFFKSAMPEGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSALSKDPNEKRDHMVLLEFVT
AAGITLGMDELYK
SEQ ID NO: 264
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPMVSKGEELFTGVVPILVELDGDVNGHKFSVSGEGEGDATYGKLTLKFICTTGKLPVPWPTLVTTVTYGV
QCFSRYPDHMKQHDFFKSAMPEGYVQERTIFFKDDGNYKTRAEVKFEGDTLVNRIELKGIDFKEDGNILGHKLEYNY
NSHNVYIMADKQKNGIKVNFKIRHNIEDGSVQLADHYQQNTPIGDGPVLLPDNHYLSTQSALSKDPNEKRDHMVLLE
FVTAAGITLGMDELYK
SEQ ID NO: 265
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPMVSKGEELFTGVVPILVELDGDVNGHKFSVSGEGEGDATYGKLTLKFICTTGKLPVPWPTLVTTVTY
GVQCFSRYPDHMKQHDFFKSAMPEGYVQERTIFFKDDGNYKTRAEVKFEGDTLVNRIELKGIDFKEDGNILGHKLEY
NYNSHNVYIMADKQKNGIKVNFKIRHNIEDGSVQLADHYQQNTPIGDGPVLLPDNHYLSTQSALSKDPNEKRDHMVL
LEFVTAAGITLGMDELYK
SEQ ID NO: 266
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPMVSKGEELFTGVVPILVELDGDVNGHKFSVSGEGEGDATYGKLTLKFICTTGKLPVPWPTLVTTV
TYGVQCFSRYPDHMKQHDFFKSAMPEGYVQERTIFFKDDGNYKTRAEVKFEGDTLVNRIELKGIDFKEDGNILGHKL
EYNYNSHNVYIMADKQKNGIKVNFKIRHNIEDGSVQLADHYQQNTPIGDGPVLLPDNHYLSTQSALSKDPNEKRDHM
VLLEFVTAAGITLGMDELYK
SEQ ID NO: 267
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPMVSKGEELFTGVVPILVELDGDVNGHKFSVSGEGEGDATYGKLTLKFICTTGKLPVPWPTLVT
TVTYGVQCFSRYPDHMKQHDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDNHYLSTQSALSKDPNEKRD
HMVLLEFVTAAGITLGMDELYK
SEQ ID NO: 268
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPMVSKGEELFTGVVPILVELDGDVNGHKFSVSGEGEGDATYGKLTLKFICTTGKLPVPWPTL
VTTVTYGVQCFSRYPDHMKQHDFFKSAMPEGYVQERTIFFKDDGNYKTRAEVKFEGDTLVNRIELKGIDFKEDGNIL
GHKLEYNYNSHNVYIMADKQKNGIKVNFKIRHNIEDGSVQLADHYQQNTPIGDGPVLLPDNHYLSTQSALSKDPNEK
RDHMVLLEFVTAAGITLGMDELYK
SEQ ID NO: 269
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPMVSKGEELFTGVVPILVELDGDVNGHKFSVSGEGEGDATYGKLTLKFICTTGKLPVPWP
TLVTTVTYGVQCFSRYPDHMKQHDFFKSAMPEGYVQERTIFFKDDGNYKTRAEVKFEGDTLVNRIELKGIDFKEDGN
ILGHKLEYNYNSHNVYIMADKQKNGIKVNFKIRHNIEDGSVQLADHYQQNTPIGDGPVLLPDNHYLSTQSALSKDPN
EKRDHMVLLEFVTAAGITLGMDELYK
SEQ ID NO: 270
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPMVSKGEELFTGVVPILVELDGDVNGHKFSVSGEGEGDATYGKLTLKFICTTGKLPVP
WPTLVTTVTYGVQCFSRYPDHMKQHDFFKSAMPEGYVQERTIFFKDDGNYKTRAEVKFEGDTLVNRIELKGIDFKED
GNILGHKLEYNYNSHNVYIMADKQKNGIKVNFKIRHNIEDGSVQLADHYQQNTPIGDGPVLLPDNHYLSTQSALSKD
PNEKRDHMVLLEFVTAAGITLGMDELYK
SEQ ID NO: 271
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPMVSKGEELFTGVVPILVELDGDVNGHKFSVSGEGEGDATYGKLTLKFICTTGKLP
VPWPTLVTTVTYGVQCFSRYPDHMKQHDFFKSAMPEGYVQERTIFFKDDGNYKTRAEVKFEGDTLVNRIELKGIDFK
EDGNILGHKLEYNYNSHNVYIMADKQKNGIKVNFKIRHNIEDGSVQLADHYQQNTPIGDGPVLLPDNHYLSTQSALS
KDPNEKRDHMVLLEFVTAAGITLGMDELYK
SEQ ID NO: 272
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPMVSKGEELFTGVVPILVELDGDVNGHKFSVSGEGEGDATYGKLTLKFICTTGK
LPVPWPTLVTTVTYGVQCFSRYPDHMKQHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLADHYQQNTPIGDGPVLLPDNHYLSTQSA
LSKDPNEKRDHMVLLEFVTAAGITLGMDELYK
SEQ ID NO: 273
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPMVSKGEELFTGVVPILVELDGDVNGHKFSVSGEGEGDATYGKLTLKFICTT
GKLPVPWPTLVTTVTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDGPVLLPDNHYLSTQ
SALSKDPNEKRDHMVLLEFVTAAGITLGMDELYK
SEQ ID NO: 274
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPXPMVSKGEELFTGVVPILVELDGDVNGHKFSVSGEGEGDATYGKLTLKFIC
TTGKLPVPWPTLVTTVTYGVQCFSRYPDHMKQHDFFKSAMPEGYVQERTIFFKDDGNYKTRAEVKFEGDTLVNRIEL
KGIDFKEDGNILGHKLEYNYNSHNVYIMADKQKNGIKVNFKIRHNIEDGSVQLADHYQQNTPIGDGPVLLPDNHYLS
TQSALSKDPNEKRDHMVLLEFVTAAGITLGMDELYK
SEQ ID NO: 275
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSEAAAKGGGGSMVSKGEELFTGVVPILVELDGDVNGHKFSVSGEGEGDATYGKLTLKFICTTGKLPVPWPTLV
TTVTYGVQCFSRYPDHMKQHDFFKSAMPEGYVQERTIFFKDDGNYKTRAEVKFEGDTLVNRIELKGIDFKEDGNILG
HKLEYNYNSHNVYIMADKQKNGIKVNFKIRHNIEDGSVQLADHYQQNTPIGDGPVLLPDNHYLSTQSALSKDPNEKR
DHMVLLEFVTAAGITLGMDELYK

Table V.8 provides chimera fusion proteins between LbCas12a variant amino acid sequences (E795L), linker amino acid sequences (underlined), and mCherry amino acid sequences (bolded, italicized).

TABLE V.8
LbCas12a(E795L)-Linker-mCherry Chimeras

SEQ ID NO: 276
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GSAGSAAGSGEFMVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGPV
MQKKTMGWEASSERMYPEDGALKGEIKQRLKLKDGGHYDEAVKTTYKAKKPVQLPGAYNVNIKLDITSHNEDYTIV
EQYEREAGRHSTGGMDELYK
SEQ ID NO: 277
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSGGGGSGGGGSGGGGSMVSKGEEDNMAIIKEFMRFKVHMEGSVNGHEFEIEGEGEGRPYEGTQTAKLKVTKG
GPLPFAWDILSPQFMYGSKAYVKHPADIPDYLKLSFPEGFKWERVMNFEDGGVVTVTQDSSLQDGEFIYKVKLRGT
NFNFPSDGPVMQKKTMGWEASSERMYPEDGALKGEIKQRLKLKDGGHYDEAVKTTYKAKKPVQLPGAYNVNIKLDI
TSHNEDYTIVEQYEREAGRHSTGGMDELYK
SEQ ID NO: 278
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMESEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKAMVSKGEEDNMAIIKEFMRFKVHMEGSVNGHEFEIEGEGEGRPYEGTQTAKLKVTK
GGPLPFAWDILSPQFMYGSKAYVKHPADIPDYLKLSFPEGFKWERVMNFEDGGVVTVTQDSSLQDGEFIYKVKLRGT
NFNFPSDGPVMQKKTMGWEASSERMYPEDGALKGEIKQRLKLKDGGHYDEAVKTTYKAKKPVQLPGAYNVNIKLDI
TSHNEDYTIVEQYEREAGRHSTGGMDELYK
SEQ ID NO: 279
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
AEAAAKEAAAKEAAAKEAAAKALEAEAAAKEAAAKEAAAKEAAAKAMVSKGEEDNMAIIKEFMRFKVHMEGSVNGHE
FEIEGEGEGRPYEGTQTAKLKVTKGGPLPFAWDILSPQFMYGSKAYVKHPADIPDYLKLSFPEGFKWERVMNFEDGG
VVTVTQDSSLQDGEFIYKVKLRGTNFNFPSDGPVMQKKTMGWEASSERMYPEDGALKGEIKQRLKLKDGGHYDEAVK
TTYKAKKPVQLPGAYNVNIKLDITSHNEDYTIVEQYEREAGRHSTGGMDELYK
SEQ ID NO: 280
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
LEAEAAAKEAAAKEAAAKEAAAKALEAEAAAKEAAAKEAAAKEAAAKALEMVSKGEEDNMAIIKEFMRFKVHMEGSV
NGHEFEIEGEGEGRPYEGTQTAKLKVTKGGPLPFAWDILSPQFMYGSKAYVKHPADIPDYLKLSFPEGFKWERVMNF
EDGGVVTVTQDSSLQDGEFIYKVKLRGTNFNFPSDGPVMQKKTMGWEASSERMYPEDGALKGEIKQRLKLKDGGHYD
EAVKTTYKAKKPVQLPGAYNVNIKLDITSHNEDYTIVEQYEREAGRHSTGGMDELYK
SEQ ID NO: 281
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAMVSKGEEDNMAIIKEFMRFKVHMEGSVNGHEFEIEGEGEGRPYEGTQTAKLKVTKGGPLPFAWDILSPQFMYGS
KAYVKHPADIPDYLKLSFPEGFKWERVMNFEDGGVVTVTQDSSLQDGEFIYKVKLRGTNFNFPSDGPVMQKKTMGWE
ASSERMYPEDGALKGEIKQRLKLKDGGHYDEAVKTTYKAKKPVQLPGAYNVNIKLDITSHNEDYTIVEQYEREAGRH
STGGMDELYK
SEQ ID NO: 282
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAMVSKGEEDNMAIIKEFMRFKVHMEGSVNGHEFEIEGEGEGRPYEGTQTAKLKVTKGGPLPFAWDILSPQFMY
GSKAYVKHPADIPDYLKLSFPEGFKWERVMNFEDGGVVTVTQDSSLQDGEFIYKVKLRGTNFNFPSDGPVMQKKTMG
WEASSERMYPEDGALKGEIKQRLKLKDGGHYDEAVKTTYKAKKPVQLPGAYNVNIKLDITSHNEDYTIVEQYEREAG
RHSTGGMDELYK
SEQ ID NO: 283
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAMVSKGEEDNMAIIKEFMRFKVHMEGSVNGHEFEIEGEGEGRPYEGTQTAKLKVTKGGPLPFAWDILSPQF
MYGSKAYVKHPADIPDYLKLSFPEGFKWERVMNFEDGGVVTVTQDSSLQDGEFIYKVKLRGTNFNFPSDGPVMQKKT
MGWEASSERMYPEDGALKGEIKQRLKLKDGGHYDEAVKTTYKAKKPVQLPGAYNVNIKLDITSHNEDYTIVEQYERE
AGRHSTGGMDELYK
SEQ ID NO: 284
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAMVSKGEEDNMAIIKEFMRFKVHMEGSVNGHEFEIEGEGEGRPYEGTQTAKLKVTKGGPLPFAWDILSP
QFMYGSKAYVKHPADIPDYLKLSFPEGFKWERVMNFEDGGVVTVTQDSSLQDGEFIYKVKLRGTNFNFPSDGPVMQK
KTMGWEASSERMYPEDGALKGEIKQRLKLKDGGHYDEAVKTTYKAKKPVQLPGAYNVNIKLDITSHNEDYTIVEQYE
REAGRHSTGGMDELYK
SEQ ID NO: 285
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGFTTAFTGE
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAMVSKGEEDNMAIIKEFMRFKVHMEGSVNGHEFEIEGEGEGRPYEGTQTAKLKVTKGGPLPFAWDIL
SPQFMYGSKAYVKHPADIPDYLKLSFPEGFKWERVMNFEDGGVVTVTQDSSLQDGEFIYKVKLRGTNFNFPSDGPVM
QKKTMGWEASSERMYPEDGALKGEIKQRLKLKDGGHYDEAVKTTYKAKKPVQLPGAYNVNIKLDITSHNEDYTIVEQ
YEREAGRHSTGGMDELYK
SEQ ID NO: 286
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAMVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFNFPSDGP
VMQKKTMGWEASSERMYPEDGALKGEIKQRLKLKDGGHYDEAVKTTYKAKKPVQLPGAYNVNIKLDITSHNEDYTIV
EQYEREAGRHSTGGMDELYK
SEQ ID NO: 287
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMESEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAMVSKGEEDNMAIIKEFMRFKVHMEGSVNGHEFEIEGEGEGRPYEGTQTAKLKVTKGGPLPFA
WDILSPQFMYGSKAYVKHPADIPDYLKLSFPEGFKWERVMNFEDGGVVTVTQDSSLQDGEFIYKVKLRGTNFNFPSD
GPVMQKKTMGWEASSERMYPEDGALKGEIKQRLKLKDGGHYDEAVKTTYKAKKPVQLPGAYNVNIKLDITSHNEDYT
IVEQYEREAGRHSTGGMDELYK
SEQ ID NO: 288
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAMVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLQDGEFIYKVKLRGTNFNFP
SDGPVMQKKTMGWEASSERMYPEDGALKGEIKQRLKLKDGGHYDEAVKTTYKAKKPVQLPGAYNVNIKLDITSHNED
YTIVEQYEREAGRHSTGGMDELYK
SEQ ID NO: 289
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAMVSKGEEDNMAIIKEFMRFKVHMEGSVNGHEFEIEGEGEGRPYEGTQTAKLKVTKGGP
LPFAWDILSPQFMYGSKAYVKHPADIPDYLKLSFPEGFKWERVMNFEDGGVVTVTQDSSLQDGEFIYKVKLRGTNFN
FPSDGPVMQKKTMGWEASSERMYPEDGALKGEIKQRLKLKDGGHYDEAVKTTYKAKKPVQLPGAYNVNIKLDITSHN
EDYTIVEQYEREAGRHSTGGMDELYK
SEQ ID NO: 290
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAMVSKGEEDNMAIIKEFMRFKVHMEGSVNGHEFEIEGEGEGRPYEGTQTAKLKVTKG
GPLPFAWDILSPQFMYGSKAYVKHPADIPDYLKLSFPEGFKWERVMNFEDGGVVTVTQDSSLQDGEFIYKVKLRGTN
FNFPSDGPVMQKKTMGWEASSERMYPEDGALKGEIKQRLKLKDGGHYDEAVKTTYKAKKPVQLPGAYNVNIKLDITS
HNEDYTIVEQYEREAGRHSTGGMDELYK
SEQ ID NO: 291
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAMVSKGEEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMNFEDGGVVTVTQDSSLQDGEFIYKVKLRG
TNFNFPSDGPVMQKKTMGWEASSERMYPEDGALKGEIKQRLKLKDGGHYDEAVKTTYKAKKPVQLPGAYNVNIKLDI
TSHNEDYTIVEQYEREAGRHSTGGMDELYK
SEQ ID NO: 292
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMFSEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAMVSKGEEDNMAIIKEFMRFKVHMEGSVNGHEFEIEGEGEGRPYEGTQTAKLK
VTKGGPLPFAWDILSPQFMYGSKAYVKHPADIPDYLKLSFPEGFKWERVMNFEDGGVVTVTQDSSLQDGEFIYKVKL
RGTNFNFPSDGPVMQKKTMGWEASSERMYPEDGALKGEIKQRLKLKDGGHYDEAVKTTYKAKKPVQLPGAYNVNIKL
DITSHNEDYTIVEQYEREAGRHSTGGMDELYK
SEQ ID NO: 293
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGETTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAMVSKGEEDNMAIIKEFMRFKVHMEGSVNGHEFEIEGEGEGRPYEGTQTAK
LKVTKGGPLPFAWDILSPQFMYGSKAYVKHPADIPDYLKLSFPEGFKWERVMNFEDGGVVTVTQDSSLQDGEFIYKV
KLRGTNFNFPSDGPVMQKKTMGWEASSERMYPEDGALKGEIKQRLKLKDGGHYDEAVKTTYKAKKPVQLPGAYNVNI
KLDITSHNEDYTIVEQYEREAGRHSTGGMDELYK
SEQ ID NO: 294
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
APAPAPAPAPAPAPAPAPAPAPAPAPAPAMVSKGEEDNMAIIKEFMRFKVHMEGSVNGHEFEIEGEGEGRPYEGTQT
AKLKVTKGGPLPFAWDILSPQFMYGSKAYVKHPADIPDYLKLSFPEGFKWERVMNFEDGGVVTVTQDSSLQDGEFIY
KVKLRGTNFNFPSDGPVMQKKTMGWEASSERMYPEDGALKGEIKQRLKLKDGGHYDEAVKTTYKAKKPVQLPGAYNV
NIKLDITSHNEDYTIVEQYEREAGRHSTGGMDELYK
SEQ ID NO: 295
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLIQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPMVSKGEEDNMAIIKEFMRFKVHMEGSVNGHEFEIEGEGEGRPYEGTQTAKLKVTKGGPLPFAWDILSPQFMYGSK
AYVKHPADIPDYLKLSFPEGFKWERVMNFEDGGVVTVTQDSSLQDGEFIYKVKLRGTNFNFPSDGPVMQKKTMGWEA
SSERMYPEDGALKGEIKQRLKLKDGGHYDEAVKTTYKAKKPVQLPGAYNVNIKLDITSHNEDYTIVEQYEREAGRHS
TGGMDELYK
SEQ ID NO: 296
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPMVSKGEEDNMAIIKEFMRFKVHMEGSVNGHEFEIEGEGEGRPYEGTQTAKLKVTKGGPLPFAWDILSPQFMYG
SKAYVKHPADIPDYLKLSFPEGFKWERVMNFEDGGVVTVTQDSSLQDGEFIYKVKLRGTNFNFPSDGPVMQKKTMGW
EASSERMYPEDGALKGEIKQRLKLKDGGHYDEAVKTTYKAKKPVQLPGAYNVNIKLDITSHNEDYTIVEQYEREAGR
HSTGGMDELYK
SEQ ID NO: 297
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPMVSKGEEDNMAIIKEFMRFKVHMEGSVNGHEFEIEGEGEGRPYEGTQTAKLKVTKGGPLPFAWDILSPQFM
YGSKAYVKHPADIPDYLKLSFPEGFKWERVMNFEDGGVVTVTQDSSLQDGEFIYKVKLRGTNFNFPSDGPVMQKKTM
GWEASSERMYPEDGALKGEIKQRLKLKDGGHYDEAVKTTYKAKKPVQLPGAYNVNIKLDITSHNEDYTIVEQYEREA
GRHSTGGMDELYK
SEQ ID NO: 298
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPMVSKGEEDNMAIIKEFMRFKVHMEGSVNGHEFEIEGEGEGRPYEGTQTAKLKVTKGGPLPFAWDILSPQ
FMYGSKAYVKHPADIPDYLKLSFPEGFKWERVMNFEDGGVVTVTQDSSLQDGEFIYKVKLRGTNFNFPSDGPVMQKK
TMGWEASSERMYPEDGALKGEIKQRLKLKDGGHYDEAVKTTYKAKKPVQLPGAYNVNIKLDITSHNEDYTIVEQYER
EAGRHSTGGMDELYK
SEQ ID NO: 299
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPMVSKGEEDNMAIIKEFMRFKVHMEGSVNGHEFEIEGEGEGRPYEGTQTAKLKVTKGGPLPFAWDILS
PQFMYGSKAYVKHPADIPDYLKLSFPEGFKWERVMNFEDGGVVTVTQDSSLQDGEFIYKVKLRGTNFNFPSDGPVMQ
KKTMGWEASSERMYPEDGALKGEIKQRLKLKDGGHYDEAVKTTYKAKKPVQLPGAYNVNIKLDITSHNEDYTIVEQY
EREAGRHSTGGMDELYK
SEQ ID NO: 300
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXMVSKGEEDNMAIIKEFMRFKVHMEGSVNGHEFEIEGEGEGRPYEGTQTAKLKVTKGGPLPFAWDIL
SPQFMYGSKAYVKHPADIPDYLKLSFPEGFKWERVMNFEDGGVVTVTQDSSLQDGEFIYKVKLRGTNFNFPSDGPVM
QKKTMGWEASSERMYPEDGALKGEIKQRLKLKDGGHYDEAVKTTYKAKKPVQLPGAYNVNIKLDITSHNEDYTIVEQ
YEREAGRHSTGGMDELYK
SEQ ID NO: 301
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGETTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPMVSKGEEDNMAIIKEFMRFKVHMEGSVNGHEFEIEGEGEGRPYEGTQTAKLKVTKGGPLPFAW
DILSPQFMYGSKAYVKHPADIPDYLKLSFPEGFKWERVMNFEDGGVVTVTQDSSLQDGEFIYKVKLRGTNFNFPSDG
PVMQKKTMGWEASSERMYPEDGALKGEIKQRLKLKDGGHYDEAVKTTYKAKKPVQLPGAYNVNIKLDITSHNEDYTI
VEQYEREAGRHSTGGMDELYK
SEQ ID NO: 302
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPMVSKGEEDNMAIIKEFMRFKVHMEGSVNGHEFEIEGEGEGRPYEGTQTAKLKVTKGGPLPF
AWDILSPQFMYGSKAYVKHPADIPDYLKLSFPEGFKWERVMNFEDGGVVTVTQDSSLQDGEFIYKVKLRGTNFNFPS
DGPVMQKKTMGWEASSERMYPEDGALKGEIKQRLKLKDGGHYDEAVKTTYKAKKPVQLPGAYNVNIKLDITSHNEDY
TIVEQYEREAGRHSTGGMDELYK
SEQ ID NO: 303
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPMVSKGEEDNMAIIKEFMRFKVHMEGSVNGHEFEIEGEGEGRPYEGTQTAKLKVTKGGPL
PFAWDILSPQFMYGSKAYVKHPADIPDYLKLSFPEGFKWERVMNFEDGGVVTVTQDSSLQDGEFIYKVKLRGTNFNF
PSDGPVMQKKTMGWEASSERMYPEDGALKGEIKQRLKLKDGGHYDEAVKTTYKAKKPVQLPGAYNVNIKLDITSHNE
DYTIVEQYEREAGRHSTGGMDELYK
SEQ ID NO: 304
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLIQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPMVSKGEEDNMAIIKEFMRFKVHMEGSVNGHEFEIEGEGEGRPYEGTQTAKLKVTKGG
PLPFAWDILSPQFMYGSKAYVKHPADIPDYLKLSFPEGFKWERVMNFEDGGVVTVTQDSSLQDGEFIYKVKLRGTNF
NFPSDGPVMQKKTMGWEASSERMYPEDGALKGEIKQRLKLKDGGHYDEAVKTTYKAKKPVQLPGAYNVNIKLDITSH
NEDYTIVEQYEREAGRHSTGGMDELYK
SEQ ID NO: 305
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPMVSKGEEDNMAIIKEFMRFKVHMEGSVNGHEFEIEGEGEGRPYEGTQTAKLKVTK
GGPLPFAWDILSPQFMYGSKAYVKHPADIPDYLKLSFPEGFKWERVMNFEDGGVVTVTQDSSLQDGEFIYKVKLRGT
NFNFPSDGPVMQKKTMGWEASSERMYPEDGALKGEIKQRLKLKDGGHYDEAVKTTYKAKKPVQLPGAYNVNIKLDIT
SHNEDYTIVEQYEREAGRHSTGGMDELYK
SEQ ID NO: 306
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGE
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPMVSKGEEDNMAIIKEFMRFKVHMEGSVNGHEFEIEGEGEGRPYEGTQTAKLKV
TKGGPLPFAWDILSPQFMYGSKAYVKHPADIPDYLKLSFPEGFKWERVMNFEDGGVVTVTQDSSLQDGEFIYKVKLR
GTNFNFPSDGPVMQKKTMGWEASSERMYPEDGALKGEIKQRLKLKDGGHYDEAVKTTYKAKKPVQLPGAYNVNIKLD
ITSHNEDYTIVEQYEREAGRHSTGGMDELYK
SEQ ID NO: 307
MSKLEKFTNCYSLSKTLRFKAIPVGKTQENIDNKRLLVEDEKRAEDYKGVKKLLDRYYLSFINDVLHSIKLKNLNNY
ISLERKKTRTEKENKELENLEINLRKEIAKAFKGNEGYKSLFKKDIIETILPEFLDDKDEIALVNSENGETTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYISDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXMVSKGEEDNMAIIKEFMRFKVHMEGSVNGHEFEIEGEGEGRPYEGTQTAKLK
VTKGGPLPFAWDILSPQFMYGSKAYVKHPADIPDYLKLSFPEGFKWERVMNFEDGGVVTVTQDSSLQDGEFIYKVKL
RGTNFNFPSDGPVMQKKTMGWEASSERMYPEDGALKGEIKQRLKLKDGGHYDEAVKTTYKAKKPVQLPGAYNVNIKL
DITSHNEDYTIVEQYEREAGRHSTGGMDELYK
SEQ ID NO: 308
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRFSEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
XPXPXPXPXPXPXPXPXPXPXPXPXPXPMVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKWERVMNFEDGGVVTVTQDSSLQDGEFIYK
VKLRGTNFNFPSDGPVMQKKTMGWEASSERMYPEDGALKGEIKQRLKLKDGGHYDEAVKTTYKAKKPVQLPGAYNVN
IKLDITSHNEDYTIVEQYEREAGRHSTGGMDELYK
SEQ ID NO: 309
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKH
GGGGSEAAAKGGGGSMVSKGEEDNMAIIKEFMRFKVHMEGSVNGHEFEIEGEGEGRPYEGTQTAKLKVTKGGPLPFA
WDILSPQFMYGSKAYVKHPADIPDYLKLSFPEGFKWERVMNFEDGGVVTVTQDSSLQDGEFIYKVKLRGTNFNFPSD
GPVMQKKTMGWEASSERMYPEDGALKGEIKQRLKLKDGGHYDEAVKTTYKAKKPVQLPGAYNVNIKLDITSHNEDYT
IVEQYEREAGRHSTGGMDELYK

Table V.9 provides the LbCas12a protein variant (E795L) amino acid sequence.

TABLE V.9
LbCas12a protein variant (E795L) amino
acid sequence

SEQ ID NO: 310 (LbCas12a (E795L) Protein)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSENGETTA
FTGFFDNRENMESEEAKSTSIAFRCINENLTRYISNMDIFEKVDAIFDKH
EVQEIKEKILNSDYDVEDFFEGEFFNEVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYLLHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH

Table V.10 provides chimera fusion proteins between Cas protein amino acid sequences to either eGFP or mCherry amino acid sequences (bolded, italicized) without an intervening linker polypeptide.

TABLE V.10
Cas Protein-Fluorescent Protein Chimeras

SEQ ID NO: 311 (Cas9 Protein-eGFP)
MDKKYSIGLDIGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKINRKVIVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLIKAERGGLSELD
KAGFIKRQLVETRQITKHVAQILDSRMNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYTSTKEVLDATLIHQSITGLYETRIDLSQLGGD
MVSKGEELFTGVVPILVELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDGNILGHKLEYNYNSHNVY
IMADKQKNGIKVNFKIRHNIEDGSVQLADHYQQNTPIGDGPVLLPDNHYLSTQSALSKDPNEKRDHMVLLEFVTAA
GITLGMDELYK
SEQ ID NO: 312 (AsCas12a Protein-eGFP)
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRIDNLTDAINKRHAEIYKGLFKAELFNGKVLKQLGTVTTTEH
ENALLRSFDKETTYFSGFYENRKNVFSAEDISTAIPHRIVQDNFPKFKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRNMV
SKGEEDNMAIIKEFMRFKVHMEGSVNGHEFEIEGEGEGRPYEGTQTAKLKVTKGGPLPFAWDILSPQFMYGSKAYVK
HPADIPDYLKLSFPEGFKWERVMNFEDGGVVTVTQDSSLQDGEFIYKVKLRGTNFPSDGPVMQKKTMGWEASSERMY
PEDGALKGEIKQRLKLKDGGHYDAEVKTTYKAKKPVQLPGAYNVNIKLDITSHNEDYTIVEQYERAEGRHSTGGMDE
LYK
SEQ ID NO: 313 (LbCas12a Protein-eGFP)
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMESEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRFSEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKHMVSK
GEELFTGVVPILVELDGDVNGHKFSVSGEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEKRDHMVLLEFVTAAGITLGMD
ELYK
SEQ ID NO: 314 (LbCas12a (E795L) Protein-eGFP)
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSENGETTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKHMVSK
GEELFTGVVPILVELDGDVNGHKFSVSGEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEKRDHMVLLEFVTAAGITLGMD
ELYK
SEQ ID NO: 315 (Cas9 Protein-mCherry)
MDKKYSIGLDIGTNSVGWAVITDEYKVPSKKEKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPINASGVDAKAILSARLSKSRRLENLIAQ
LPGEKKNGLFGNLIALSLGLIPNEKSNFDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTKVKYVTE
GMRKPAFLSGEQKKAIVDLLFKINRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQLGGD
MVSKGEEDNMAIIKEFMRFKVHMEGSVNGHEFEIEGEGEGRPYEGTQTAKLKVTKGGPLPFAWDILSPQFMYGSKA
YVKHPADIPDYLKLSFPEGFKWERVMNFEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVNIKLDITSHNEDYTIVEQYERAEGRHST
GGMDELYK
SEQ ID NO: 316 (AsCas12a Protein-mCherry)
MTQFEGFTNLYQVSKTLRFELIPQGKTLKHIQEQGFIEEDKARNDHYKELKPIIDRIYKTYADQCLQLVQLDWENLS
AAIDSYRKEKTEETRNALIEEQATYRNAIHDYFIGRTDNLTDAINKRHAEIYKGLFKAELFNGKVLKQLGTVTTTEH
ENALLRSFDKETTYFSGFYENRKNVESAEDISTAIPHRIVQDNFPKEKENCHIFTRLITAVPSLREHFENVKKAIGI
FVSTSIEEVFSFPFYNQLLTQTQIDLYNQLLGGISREAGTEKIKGLNEVLNLAIQKNDETAHIIASLPHRFIPLFKQ
ILSDRNTLSFILEEFKSDEEVIQSFCKYKTLLRNENVLETAEALFNELNSIDLTHIFISHKKLETISSALCDHWDTL
RNALYERRISELTGKITKSAKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREALCKWIDFTRDFLSKYTKTTSI
DLSSLRPSSQYKDLGEYYAELNPLLYHISFQRIAEKEIMDAVETGKLYLFQIYNKDFAKGHHGKPNLHTLYWTGLFS
PENLAKTSIKLNGQAELFYRPKSRMKRMAHRLGEKMLNKKLKDQKTPIPDTLYQELYDYVNHRLSHDLSDEARALLP
NVITKEVSHEIIKDRRFTSDKEFFHVPITLNYQAANSPSKFNQRVNAYLKEHPETPIIGIDRGERNLIYITVIDSTG
KILEQRSLNTIQQFDYQKKLDNREKERVAARQAWSVVGTIKDLKQGYLSQVIHEIVDLMIHYQAVVVLENLNFGFKS
KRTGIAEKAVYQQFEKMLIDKLNCLVLKDYPAEKVGGVLNPYQLTDQFTSFAKMGTQSGELFYVPAPYTSKIDPLTG
FVDPFVWKTIKNHESRKHFLEGFDFLHYDVKTGDFILHFKMNRNLSFQRGLPGEMPAWDIVFEKNETQFDAKGTPFI
AGKRIVPVIENHRFTGRYRDLYPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLKLQNGISNQDWLAYIQELRNMV
SKGEEDNMAIIKEFMRFKVHMEGSVNGHEFEIEGEGEGRPYEGTQTAKLKVTKGGPLPFAWDILSPQFMYGSKAYVK
HPADIPDYLKLSFPEGFKWERVMNFEDGGVVTVTQDSSLQDGEFIYKVKLRGTNFPSDGPVMQKKTMGWEASSERMY
PEDGALKGEIKQRLKLKDGGHYDAEVKTTYKAKKPVQLPGAYNVNIKLDITSHNEDYTIVEQYERAEGRHSTGGMDE
LYK
SEQ ID NO: 317 (LbCas12a Protein-mCherry)
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMESEEAKSTSIAFRCINENLTRYISNMDIFEKVDAIFDKHEVQEIKEKILNSDYDVEDFFEGEFFNFVLTQ
EGIDVYNAIIGGFVTESGEKIKGLNEYINLYNQKTKQKLPKFKPLYKQVLSDRESLSFYGEGYTSDEEVLEVFRNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDFNFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKITTLSYDVYKDKRESEDQYELHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVEDWEEVCLTSAYKELENKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKHMVSK
GEEDNMAIIKEFMRFKVHMEGSVNGHEFEIEGEGEGRPYEGTQTAKLKVTKGGPLPFAWDILSPQFMYGSKAYVKHP
ADIPDYLKLSFPEGFKWERVMNFEDGGVVTVTQDSSLQDGEFIYKVKLRGTNFPSDGPVMQKKTMGWEASSERMYPE
DGALKGEIKQRLKLKDGGHYDAEVKTTYKAKKPVQLPGAYNVNIKLDITSHNEDYTIVEQYERAEGRHSTGGMDELY
K
SEQ ID NO: 318 (LbCas12a (E795L) Protein-mCherry)
MSKLEKFTNCYSLSKTLRFKAIPVGKTQENIDNKRLLVEDEKRAEDYKGVKKLLDRYYLSFINDVLHSIKLKNLNNY
ISLFRKKTRTEKENKELENLEINLRKEIAKAFKGNEGYKSLFKKDIIETILPEFLDDKDEIALVNSFNGFTTAFTGF
FDNRENMFSEEAKSTSIAFRCINENLTRYISNMDIFEKVDAIFDKHEVQEIKEKILNSDYDVEDFFEGEFFNEVLTQ
EGIDVYNAIIGGFVTESGEKIKGLNEYINLYNQKTKQKLPKFKPLYKQVLSDRESLSFYGEGYTSDEEVLEVERNTL
NKNSEIFSSIKKLEKLFKNFDEYSSAGIFVKNGPAISTISKDIFGEWNVIRDKWNAEYDDIHLKKKAVVTEKYEDDR
RKSFKKIGSFSLEQLQEYADADLSVVEKLKEIIIQKVDEIYKVYGSSEKLFDADFVLEKSLKKNDAVVAIMKDLLDS
VKSFENYIKAFFGEGKETNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFSKKWMAYYNPSEDIQKIY
KNGTFKKGDMFNLNDCHKLIDFFKDSISRYPKWSNAYDENFSETEKYKDIAGFYREVEEQGYKVSFESASKKEVDKL
VEEGKLYMFQIYNKDFSDKSHGTPNLHTMYFKLLFDENNHGQIRLSGGAELFMRRASLKKEELVVHPANSPIANKNP
DNPKKTTTLSYDVYKDKRESEDQYLLHIPIAINKCPKNIFKINTEVRVLLKHDDNPYVIGIDRGERNLLYIVVVDGK
GNIVEQYSLNEIINNFNGIRIKTDYHSLLDKKEKERFEARQNWTSIENIKELKAGYISQVVHKICELVEKYDAVIAL
EDLNSGFKNSRVKVEKQVYQKFEKMLIDKLNYMVDKKSNPCATGGALKGYQITNKFESFKSMSTQNGFIFYIPAWLT
SKIDPSTGFVNLLKTKYTSIADSKKFISSFDRIMYVPEEDLFEFALDYKNFSRTDADYIKKWKLYSYGNRIRIFRNP
KKNNVFDWEEVCLTSAYKELFNKYGINYQQGDIRALLCEQSDKAFYSSFMALMSLMLQMRNSITGRTDVDFLISPVK
NSDGIFYDSRNYEAQENAILPKNADANGAYNIARKVLWAIGQFKKAEDEKLDKVKIAISNKEWLEYAQTSVKHMVSK
GEEDNMAIIKEFMRFKVHMEGSVNGHEFEIEGEGEGRPYEGTQTAKLKVTKGGPLPFAWDILSPQFMYGSKAYVKHP
ADIPDYLKLSFPEGFKWERVMNFEDGGVVTVTQDSSLQDGEFIYKVKLRGTNFPSDGPVMQKKTMGWEASSERMYPE
DGALKGEIKQRLKLKDGGHYDAEVKTTYKAKKPVQLPGAYNVNIKLDITSHNEDYTIVEQYERAEGRHSTGGMDELY
K

### Exemplary DNA Sequences Encoding Cas9 Protein Chimeras

(Wild-type Cas9 protein DNA sequence)
SEQ ID NO: 347
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGAT
(Cas9-GSAGSAAGSGEF-(SEQ ID NO: 6)-eGFP chimera DNA sequence)
SEQ ID NO: 348
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGTAGCGCAGGTAGTGCAGCAGGTAGCGGTGAATTTATGGTTAGCAAAGGTGAA
GAACTGTTTACCGGCGTTGTGCCGATTCTGGTGGAACTGGATGGTGATGTGAATGGCCATAAATTTAGCGTTagcGG
CGAAGGCGAAGGTGATGCGACCtatGGTAAACTGACCCTGAAATTTATTTGCACCACCGGTAAACTGCCGGTTCCGT
GGCCGACCCTGGTGACCACCCTGACCTATGGCGTTCAGTGCTTTAGCCGCTATCCGGATCATATGAAAcagCATGAT
TTCTTTAAAAGCGCGATGCCGGAAGGCTATGTGCAGGAACGTACCATTtttTTCAAAGATGATGGCaacTATAAAAC
CCGTGCGGAAGTTAAATTTGAAGGCGATACCCTGGTGAACCGCATTGAACTGAAAGGTATTGATTTTAAAGAAGATG
GCAACATTCTGGGTCATAAACTGGAATATAATtacAACAGCCATAATGTGTATATTatgGCCGATAAACAGAAAAAT
GGCATCAAAgtgAACTTTAAAATCCGTCACAACattGAAGATGGTAGCGTGCAGCTGGCGGATCATTATCAGCAGAA
TACCCCGATTGGTGATGGCCCGGTGCTGCTGCCGGATAATCATTATCTGAGCACCCAGAGCgcgCTGAGCAAAGATC
CGAATGAAAAACGTGATCATATGGTGCTGCTGGAATTTGTTACCGCCGCGGGCATTACCctgGGTATGGATGAACTG
TATAAA
(Cas9-(GGGGS)₄-(SEQ ID NO: 7)eGFP chimera DNA sequence)
SEQ ID NO: 349
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGAGGCGGAGGATCAGGCGGTGGCGGAAGTGGTGGAGGTGGGTCGGGAGGTGGT
GGTAGCATGGTTAGCAAAGGTGAAGAACTGTTTACCGGCGTTGTGCCGATTCTGGTGGAACTGGATGGTGATGTGAA
TGGCCATAAATTTAGCGTTagcGGCGAAGGCGAAGGTGATGCGACCtatGGTAAACTGACCCTGAAATTTATTTGCA
CCACCGGTAAACTGCCGGTTCCGTGGCCGACCCTGGTGACCACCCTGACCTATGGCGTTCAGTGCTTTAGCCGCTAT
CCGGATCATATGAAAcagCATGATTTCTTTAAAAGCGCGATGCCGGAAGGCTATGTGCAGGAACGTACCATTtttTT
CAAAGATGATGGCaacTATAAAACCCGTGCGGAAGTTAAATTTGAAGGCGATACCCTGGTGAACCGCATTGAACTGA
AAGGTATTGATTTTAAAGAAGATGGCAACATTCTGGGTCATAAACTGGAATATAATtacAACAGCCATAATGTGTAT
ATTatgGCCGATAAACAGAAAAATGGCATCAAAgtgAACTTTAAAATCCGTCACAACattGAAGATGGTAGCGTGCA
GCTGGCGGATCATTATCAGCAGAATACCCCGATTGGTGATGGCCCGGTGCTGCTGCCGGATAATCATTATCTGAGCA
CCCAGAGCgcgCTGAGCAAAGATCCGAATGAAAAACGTGATCATATGGTGCTGCTGGAATTTGTTACCGCCGCGGGC
ATTACCctgGGTATGGATGAACTGTATAAA
(Cas9-GGGGSEAAAKGGGGS(SEQ ID NO: 37)-eGFP chimera DNA sequence)
SEQ ID NO: 350
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGAGGCGGAGGGTCGGAAGCGGCAGCAAAAGGAGGCGGAGGAAGTATGGTTAGC
AAAGGTGAAGAACTGTTTACCGGCGTTGTGCCGATTCTGGTGGAACTGGATGGTGATGTGAATGGCCATAAATTTAG
CGTTagcGGCGAAGGCGAAGGTGATGCGACCtatGGTAAACTGACCCTGAAATTTATTTGCACCACCGGTAAACTGC
CGGTTCCGTGGCCGACCCTGGTGACCACCCTGACCTATGGCGTTCAGTGCTTTAGCCGCTATCCGGATCATATGAAA
cagCATGATTTCTTTAAAAGCGCGATGCCGGAAGGCTATGTGCAGGAACGTACCATTtttTTCAAAGATGATGGCaa
cTATAAAACCCGTGCGGAAGTTAAATTTGAAGGCGATACCCTGGTGAACCGCATTGAACTGAAAGGTATTGATTTTA
AAGAAGATGGCAACATTCTGGGTCATAAACTGGAATATAATtacAACAGCCATAATGTGTATATTatgGCCGATAAA
CAGAAAAATGGCATCAAAgtgAACTTTAAAATCCGTCACAACattGAAGATGGTAGCGTGCAGCTGGCGGATCATTA
TCAGCAGAATACCCCGATTGGTGATGGCCCGGTGCTGCTGCCGGATAATCATTATCTGAGCACCCAGAGCgcgCTGA
GCAAAGATCCGAATGAAAAACGTGATCATATGGTGCTGCTGGAATTTGTTACCGCCGCGGGCATTACCctgGGTATG
GATGAACTGTATAAA
(Cas9-A(EAAAK)₄A-(SEQ ID NO: 8)eGFP chimera DNA sequence)
SEQ ID NO: 351
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTGAGGCCGCAGCAAAAGAGGCTGCCGCTAAGGAGGCTGCTGCAAAAGAAGCA
GCAGCGAAAGCTATGGTTAGCAAAGGTGAAGAACTGTTTACCGGCGTTGTGCCGATTCTGGTGGAACTGGATGGTGA
TGTGAATGGCCATAAATTTAGCGTTagcGGCGAAGGCGAAGGTGATGCGACCtatGGTAAACTGACCCTGAAATTTA
TTTGCACCACCGGTAAACTGCCGGTTCCGTGGCCGACCCTGGTGACCACCCTGACCTATGGCGTTCAGTGCTTTAGC
CGCTATCCGGATCATATGAAAcagCATGATTTCTTTAAAAGCGCGATGCCGGAAGGCTATGTGCAGGAACGTACCAT
TtttTTCAAAGATGATGGCaacTATAAAACCCGTGCGGAAGTTAAATTTGAAGGCGATACCCTGGTGAACCGCATTG
AACTGAAAGGTATTGATTTTAAAGAAGATGGCAACATTCTGGGTCATAAACTGGAATATAATtacAACAGCCATAAT
GTGTATATTatgGCCGATAAACAGAAAAATGGCATCAAAgtgAACTTTAAAATCCGTCACAACattGAAGATGGTAG
CGTGCAGCTGGCGGATCATTATCAGCAGAATACCCCGATTGGTGATGGCCCGGTGCTGCTGCCGGATAATCATTATC
TGAGCACCCAGAGCgcgCTGAGCAAAGATCCGAATGAAAAACGTGATCATATGGTGCTGCTGGAATTTGTTACCGCC
GCGGGCATTACCctgGGTATGGATGAACTGTATAAA
(Cas9-A(EAAAK)₄ALEA(EAAAK)₄A(SEQ ID NO: 9)-eGFP chimera DNA sequence)
SEQ ID NO: 352
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCCGAAGCAGCAGCCAAAGAAGCTGCCGCAAAGGAGGCGGCAGCTAAAGAAGCG
GCCGCTAAGGCATTGGAAGCGGAGGCGGCTGCCAAGGAGGCTGCGGCGAAAGAAGCTGCGGCCAAAGAAGCGGCTGC
AAAAGCGATGGTTAGCAAAGGTGAAGAACTGTTTACCGGCGTTGTGCCGATTCTGGTGGAACTGGATGGTGATGTGA
ATGGCCATAAATTTAGCGTTagcGGCGAAGGCGAAGGTGATGCGACCtatGGTAAACTGACCCTGAAATTTATTTGC
ACCACCGGTAAACTGCCGGTTCCGTGGCCGACCCTGGTGACCACCCTGACCTATGGCGTTCAGTGCTTTAGCCGCTA
TCCGGATCATATGAAAcagCATGATTTCTTTAAAAGCGCGATGCCGGAAGGCTATGTGCAGGAACGTACCATTtttT
TCAAAGATGATGGCaacTATAAAACCCGTGCGGAAGTTAAATTTGAAGGCGATACCCTGGTGAACCGCATTGAACTG
AAAGGTATTGATTTTAAAGAAGATGGCAACATTCTGGGTCATAAACTGGAATATAATtacAACAGCCATAATGTGTA
TATTatgGCCGATAAACAGAAAAATGGCATCAAAgtgAACTTTAAAATCCGTCACAACattGAAGATGGTAGCGTGC
AGCTGGCGGATCATTATCAGCAGAATACCCCGATTGGTGATGGCCCGGTGCTGCTGCCGGATAATCATTATCTGAGC
ACCCAGAGCgcgCTGAGCAAAGATCCGAATGAAAAACGTGATCATATGGTGCTGCTGGAATTTGTTACCGCCGCGGG
CATTACCctgGGTATGGATGAACTGTATAAA
(Cas9-LEA(EAAAK)₄ALEA(EAAAK)₄ALE(SEQ ID NO: 10)-eGFP chimera DNA sequence)
SEQ ID NO: 353
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATTTAGAGGCTGAGGCTGCTGCCAAGGAGGCTGCTGCGAAGGAAGCTGCAGCTAAA
GAGGCAGCGGCCAAAGCACTTGAGGCCGAAGCCGCCGCGAAAGAAGCCGCTGCTAAGGAGGCAGCCGCCAAAGAAGC
GGCAGCTAAAGCATTAGAAATGGTTAGCAAAGGTGAAGAACTGTTTACCGGCGTTGTGCCGATTCTGGTGGAACTGG
ATGGTGATGTGAATGGCCATAAATTTAGCGTTagcGGCGAAGGCGAAGGTGATGCGACCtatGGTAAACTGACCCTG
AAATTTATTTGCACCACCGGTAAACTGCCGGTTCCGTGGCCGACCCTGGTGACCACCCTGACCTATGGCGTTCAGTG
CTTTAGCCGCTATCCGGATCATATGAAAcagCATGATTTCTTTAAAAGCGCGATGCCGGAAGGCTATGTGCAGGAAC
GTACCATTtttTTCAAAGATGATGGCaacTATAAAACCCGTGCGGAAGTTAAATTTGAAGGCGATACCCTGGTGAAC
CGCATTGAACTGAAAGGTATTGATTTTAAAGAAGATGGCAACATTCTGGGTCATAAACTGGAATATAATtacAACAG
CCATAATGTGTATATTatgGCCGATAAACAGAAAAATGGCATCAAAgtgAACTTTAAAATCCGTCACAACattGAAG
ATGGTAGCGTGCAGCTGGCGGATCATTATCAGCAGAATACCCCGATTGGTGATGGCCCGGTGCTGCTGCCGGATAAT
CATTATCTGAGCACCCAGAGCgcgCTGAGCAAAGATCCGAATGAAAAACGTGATCATATGGTGCTGCTGGAATTTGT
TACCGCCGCGGGCATTACCctgGGTATGGATGAACTGTATAAA
(Cas9-APA-eGFP chimera DNA sequence)
SEQ ID NO: 354
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCACCTGCGATGGTTAGCAAAGGTGAAGAACTGTTTACCGGCGTTGTGCCGATT
CTGGTGGAACTGGATGGTGATGTGAATGGCCATAAATTTAGCGTTagcGGCGAAGGCGAAGGTGATGCGACCtatGG
TAAACTGACCCTGAAATTTATTTGCACCACCGGTAAACTGCCGGTTCCGTGGCCGACCCTGGTGACCACCCTGACCT
ATGGCGTTCAGTGCTTTAGCCGCTATCCGGATCATATGAAAcagCATGATTTCTTTAAAAGCGCGATGCCGGAAGGC
TATGTGCAGGAACGTACCATTtttTTCAAAGATGATGGCaacTATAAAACCCGTGCGGAAGTTAAATTTGAAGGCGA
TACCCTGGTGAACCGCATTGAACTGAAAGGTATTGATTTTAAAGAAGATGGCAACATTCTGGGTCATAAACTGGAAT
ATAATtacAACAGCCATAATGTGTATATTatgGCCGATAAACAGAAAAATGGCATCAAAgtgAACTTTAAAATCCGT
CACAACattGAAGATGGTAGCGTGCAGCTGGCGGATCATTATCAGCAGAATACCCCGATTGGTGATGGCCCGGTGCT
GCTGCCGGATAATCATTATCTGAGCACCCAGAGCgcgCTGAGCAAAGATCCGAATGAAAAACGTGATCATATGGTGC
TGCTGGAATTTGTTACCGCCGCGGGCATTACCctgGGTATGGATGAACTGTATAAA
(Cas9-APAPA(SEQ ID NO: 11)-eGFP chimera DNA sequence)
SEQ ID NO: 355
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTCCTGCACCTGCGATGGTTAGCAAAGGTGAAGAACTGTTTACCGGCGTTGTG
CCGATTCTGGTGGAACTGGATGGTGATGTGAATGGCCATAAATTTAGCGTTagcGGCGAAGGCGAAGGTGATGCGAC
CtatGGTAAACTGACCCTGAAATTTATTTGCACCACCGGTAAACTGCCGGTTCCGTGGCCGACCCTGGTGACCACCC
TGACCTATGGCGTTCAGTGCTTTAGCCGCTATCCGGATCATATGAAAcagCATGATTTCTTTAAAAGCGCGATGCCG
GAAGGCTATGTGCAGGAACGTACCATTtttTTCAAAGATGATGGCaacTATAAAACCCGTGCGGAAGTTAAATTTGA
AGGCGATACCCTGGTGAACCGCATTGAACTGAAAGGTATTGATTTTAAAGAAGATGGCAACATTCTGGGTCATAAAC
TGGAATATAATtacAACAGCCATAATGTGTATATTatgGCCGATAAACAGAAAAATGGCATCAAAgtgAACTTTAAA
ATCCGTCACAACattGAAGATGGTAGCGTGCAGCTGGCGGATCATTATCAGCAGAATACCCCGATTGGTGATGGCCC
GGTGCTGCTGCCGGATAATCATTATCTGAGCACCCAGAGCgcgCTGAGCAAAGATCCGAATGAAAAACGTGATCATA
TGGTGCTGCTGGAATTTGTTACCGCCGCGGGCATTACCctgGGTATGGATGAACTGTATAAA
(Cas9-APAPAPA(SEQ ID NO: 12)-eGFP chimera DNA sequence)
SEQ ID NO: 356
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCTCCTGCACCTGCGATGGTTAGCAAAGGTGAAGAACTGTTTACCGGC
GTTGTGCCGATTCTGGTGGAACTGGATGGTGATGTGAATGGCCATAAATTTAGCGTTagcGGCGAAGGCGAAGGTGA
TGCGACCtatGGTAAACTGACCCTGAAATTTATTTGCACCACCGGTAAACTGCCGGTTCCGTGGCCGACCCTGGTGA
CCACCCTGACCTATGGCGTTCAGTGCTTTAGCCGCTATCCGGATCATATGAAAcagCATGATTTCTTTAAAAGCGCG
ATGCCGGAAGGCTATGTGCAGGAACGTACCATTtttTTCAAAGATGATGGCaacTATAAAACCCGTGCGGAAGTTAA
ATTTGAAGGCGATACCCTGGTGAACCGCATTGAACTGAAAGGTATTGATTTTAAAGAAGATGGCAACATTCTGGGTC
ATAAACTGGAATATAATtacAACAGCCATAATGTGTATATTatgGCCGATAAACAGAAAAATGGCATCAAAgtgAAC
TTTAAAATCCGTCACAACattGAAGATGGTAGCGTGCAGCTGGCGGATCATTATCAGCAGAATACCCCGATTGGTGA
TGGCCCGGTGCTGCTGCCGGATAATCATTATCTGAGCACCCAGAGCgcgCTGAGCAAAGATCCGAATGAAAAACGTG
ATCATATGGTGCTGCTGGAATTTGTTACCGCCGCGGGCATTACCctgGGTATGGATGAACTGTATAAA
(Cas9-APAPAPAPA(SEQ ID NO: 13)-eGFP chimera DNA sequence)
SEQ ID NO: 357
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTCCTGCGCCAGCTCCTGCACCTGCGATGGTTAGCAAAGGTGAAGAACTGTTT
ACCGGCGTTGTGCCGATTCTGGTGGAACTGGATGGTGATGTGAATGGCCATAAATTTAGCGTTagcGGCGAAGGCGA
AGGTGATGCGACCtatGGTAAACTGACCCTGAAATTTATTTGCACCACCGGTAAACTGCCGGTTCCGTGGCCGACCC
TGGTGACCACCCTGACCTATGGCGTTCAGTGCTTTAGCCGCTATCCGGATCATATGAAAcagCATGATTTCTTTAAA
AGCGCGATGCCGGAAGGCTATGTGCAGGAACGTACCATTtttTTCAAAGATGATGGCaacTATAAAACCCGTGCGGA
AGTTAAATTTGAAGGCGATACCCTGGTGAACCGCATTGAACTGAAAGGTATTGATTTTAAAGAAGATGGCAACATTC
TGGGTCATAAACTGGAATATAATtacAACAGCCATAATGTGTATATTatgGCCGATAAACAGAAAAATGGCATCAAA
gtgAACTTTAAAATCCGTCACAACattGAAGATGGTAGCGTGCAGCTGGCGGATCATTATCAGCAGAATACCCCGAT
TGGTGATGGCCCGGTGCTGCTGCCGGATAATCATTATCTGAGCACCCAGAGCgcgCTGAGCAAAGATCCGAATGAAA
AACGTGATCATATGGTGCTGCTGGAATTTGTTACCGCCGCGGGCATTACCctgGGTATGGATGAACTGTATAAA
(Cas9-APAPAPAPAPA(SEQ ID NO: 14)-eGFP chimera DNA sequence)
SEQ ID NO: 358
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCCGCTCCTGCGCCAGCTCCTGCACCTGCGATGGTTAGCAAAGGTGAAGAA
CTGTTTACCGGCGTTGTGCCGATTCTGGTGGAACTGGATGGTGATGTGAATGGCCATAAATTTAGCGTTagcGGCGA
AGGCGAAGGTGATGCGACCtatGGTAAACTGACCCTGAAATTTATTTGCACCACCGGTAAACTGCCGGTTCCGTGGC
CGACCCTGGTGACCACCCTGACCTATGGCGTTCAGTGCTTTAGCCGCTATCCGGATCATATGAAAcagCATGATTTC
TTTAAAAGCGCGATGCCGGAAGGCTATGTGCAGGAACGTACCATTtttTTCAAAGATGATGGCaacTATAAAACCCG
TGCGGAAGTTAAATTTGAAGGCGATACCCTGGTGAACCGCATTGAACTGAAAGGTATTGATTTTAAAGAAGATGGCA
ACATTCTGGGTCATAAACTGGAATATAATtacAACAGCCATAATGTGTATATTatgGCCGATAAACAGAAAAATGGC
ATCAAAgtgAACTTTAAAATCCGTCACAACattGAAGATGGTAGCGTGCAGCTGGCGGATCATTATCAGCAGAATAC
CCCGATTGGTGATGGCCCGGTGCTGCTGCCGGATAATCATTATCTGAGCACCCAGAGCgcgCTGAGCAAAGATCCGA
ATGAAAAACGTGATCATATGGTGCTGCTGGAATTTGTTACCGCCGCGGGCATTACCctgGGTATGGATGAACTGTAT
AAA
(Cas9-APAPAPAPAPAPA(SEQ ID NO: 15)-eGFP chimera DNA sequence)
SEQ ID NO: 359
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCACCTGCGATGGTTAGCAAAGGT
GAAGAACTGTTTACCGGCGTTGTGCCGATTCTGGTGGAACTGGATGGTGATGTGAATGGCCATAAATTTAGCGTTag
cGGCGAAGGCGAAGGTGATGCGACCtatGGTAAACTGACCCTGAAATTTATTTGCACCACCGGTAAACTGCCGGTTC
CGTGGCCGACCCTGGTGACCACCCTGACCTATGGCGTTCAGTGCTTTAGCCGCTATCCGGATCATATGAAAcagCAT
GATTTCTTTAAAAGCGCGATGCCGGAAGGCTATGTGCAGGAACGTACCATTtttTTCAAAGATGATGGCaacTATAA
AACCCGTGCGGAAGTTAAATTTGAAGGCGATACCCTGGTGAACCGCATTGAACTGAAAGGTATTGATTTTAAAGAAG
ATGGCAACATTCTGGGTCATAAACTGGAATATAATtacAACAGCCATAATGTGTATATTatgGCCGATAAACAGAAA
AATGGCATCAAAgtgAACTTTAAAATCCGTCACAACattGAAGATGGTAGCGTGCAGCTGGCGGATCATTATCAGCA
GAATACCCCGATTGGTGATGGCCCGGTGCTGCTGCCGGATAATCATTATCTGAGCACCCAGAGCgcgCTGAGCAAAG
ATCCGAATGAAAAACGTGATCATATGGTGCTGCTGGAATTTGTTACCGCCGCGGGCATTACCctgGGTATGGATGAA
CTGTATAAA
(Cas9-APAPAPAPAPAPAPA(SEQ ID NO: 16)-eGFP chimera DNA sequence)
SEQ ID NO: 360
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCGATGGTTAGC
AAAGGTGAAGAACTGTTTACCGGCGTTGTGCCGATTCTGGTGGAACTGGATGGTGATGTGAATGGCCATAAATTTAG
CGTTagcGGCGAAGGCGAAGGTGATGCGACCtatGGTAAACTGACCCTGAAATTTATTTGCACCACCGGTAAACTGC
CGGTTCCGTGGCCGACCCTGGTGACCACCCTGACCTATGGCGTTCAGTGCTTTAGCCGCTATCCGGATCATATGAAA
cagCATGATTTCTTTAAAAGCGCGATGCCGGAAGGCTATGTGCAGGAACGTACCATTtttTTCAAAGATGATGGCaa
cTATAAAACCCGTGCGGAAGTTAAATTTGAAGGCGATACCCTGGTGAACCGCATTGAACTGAAAGGTATTGATTTTA
AAGAAGATGGCAACATTCTGGGTCATAAACTGGAATATAATtacAACAGCCATAATGTGTATATTatgGCCGATAAA
CAGAAAAATGGCATCAAAgtgAACTTTAAAATCCGTCACAACattGAAGATGGTAGCGTGCAGCTGGCGGATCATTA
TCAGCAGAATACCCCGATTGGTGATGGCCCGGTGCTGCTGCCGGATAATCATTATCTGAGCACCCAGAGCgcgCTGA
GCAAAGATCCGAATGAAAAACGTGATCATATGGTGCTGCTGGAATTTGTTACCGCCGCGGGCATTACCctgGGTATG
GATGAACTGTATAAA
(Cas9-APAPAPAPAPAPAPAPA(SEQ ID NO: 17)-eGFP chimera DNA sequence)
SEQ ID NO: 361
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCGCCAGCTCCTGCACCTGCGATG
GTTAGCAAAGGTGAAGAACTGTTTACCGGCGTTGTGCCGATTCTGGTGGAACTGGATGGTGATGTGAATGGCCATAA
ATTTAGCGTTagcGGCGAAGGCGAAGGTGATGCGACCtatGGTAAACTGACCCTGAAATTTATTTGCACCACCGGTA
AACTGCCGGTTCCGTGGCCGACCCTGGTGACCACCCTGACCTATGGCGTTCAGTGCTTTAGCCGCTATCCGGATCAT
ATGAAAcagCATGATTTCTTTAAAAGCGCGATGCCGGAAGGCTATGTGCAGGAACGTACCATTtttTTCAAAGATGA
TGGCaacTATAAAACCCGTGCGGAAGTTAAATTTGAAGGCGATACCCTGGTGAACCGCATTGAACTGAAAGGTATTG
ATTTTAAAGAAGATGGCAACATTCTGGGTCATAAACTGGAATATAATtacAACAGCCATAATGTGTATATTatgGCC
GATAAACAGAAAAATGGCATCAAAgtgAACTTTAAAATCCGTCACAACattGAAGATGGTAGCGTGCAGCTGGCGGA
TCATTATCAGCAGAATACCCCGATTGGTGATGGCCCGGTGCTGCTGCCGGATAATCATTATCTGAGCACCCAGAGCg
cgCTGAGCAAAGATCCGAATGAAAAACGTGATCATATGGTGCTGCTGGAATTTGTTACCGCCGCGGGCATTACCctg
GGTATGGATGAACTGTATAAA
(Cas9-APAPAPAPAPAPAPAPAPA(SEQ ID NO: 18)-eGFP chimera DNA sequence)
SEQ ID NO: 362
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCGCCAGCTCCTGCACCT
GCGATGGTTAGCAAAGGTGAAGAACTGTTTACCGGCGTTGTGCCGATTCTGGTGGAACTGGATGGTGATGTGAATGG
CCATAAATTTAGCGTTagcGGCGAAGGCGAAGGTGATGCGACCtatGGTAAACTGACCCTGAAATTTATTTGCACCA
CCGGTAAACTGCCGGTTCCGTGGCCGACCCTGGTGACCACCCTGACCTATGGCGTTCAGTGCTTTAGCCGCTATCCG
GATCATATGAAAcagCATGATTTCTTTAAAAGCGCGATGCCGGAAGGCTATGTGCAGGAACGTACCATTtttTTCAA
AGATGATGGCaacTATAAAACCCGTGCGGAAGTTAAATTTGAAGGCGATACCCTGGTGAACCGCATTGAACTGAAAG
GTATTGATTTTAAAGAAGATGGCAACATTCTGGGTCATAAACTGGAATATAATtacAACAGCCATAATGTGTATATT
atgGCCGATAAACAGAAAAATGGCATCAAAgtgAACTTTAAAATCCGTCACAACattGAAGATGGTAGCGTGCAGCT
GGCGGATCATTATCAGCAGAATACCCCGATTGGTGATGGCCCGGTGCTGCTGCCGGATAATCATTATCTGAGCACCC
AGAGCgcgCTGAGCAAAGATCCGAATGAAAAACGTGATCATATGGTGCTGCTGGAATTTGTTACCGCCGCGGGCATT
ACCctgGGTATGGATGAACTGTATAAA
(Cas9-APAPAPAPAPAPAPAPAPAPA(SEQ ID NO: 19)-eGFP chimera DNA sequence)
SEQ ID NO: 363
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCTCCTGCGCCAGCTCCT
GCACCTGCGATGGTTAGCAAAGGTGAAGAACTGTTTACCGGCGTTGTGCCGATTCTGGTGGAACTGGATGGTGATGT
GAATGGCCATAAATTTAGCGTTagcGGCGAAGGCGAAGGTGATGCGACCtatGGTAAACTGACCCTGAAATTTATTT
GCACCACCGGTAAACTGCCGGTTCCGTGGCCGACCCTGGTGACCACCCTGACCTATGGCGTTCAGTGCTTTAGCCGC
TATCCGGATCATATGAAAcagCATGATTTCTTTAAAAGCGCGATGCCGGAAGGCTATGTGCAGGAACGTACCATTtt
tTTCAAAGATGATGGCaacTATAAAACCCGTGCGGAAGTTAAATTTGAAGGCGATACCCTGGTGAACCGCATTGAAC
TGAAAGGTATTGATTTTAAAGAAGATGGCAACATTCTGGGTCATAAACTGGAATATAATtacAACAGCCATAATGTG
TATATTatgGCCGATAAACAGAAAAATGGCATCAAAgtgAACTTTAAAATCCGTCACAACattGAAGATGGTAGCGT
GCAGCTGGCGGATCATTATCAGCAGAATACCCCGATTGGTGATGGCCCGGTGCTGCTGCCGGATAATCATTATCTGA
GCACCCAGAGCgcgCTGAGCAAAGATCCGAATGAAAAACGTGATCATATGGTGCTGCTGGAATTTGTTACCGCCGCG
GGCATTACCctgGGTATGGATGAACTGTATAAA
(Cas9-APAPAPAPAPAPAPAPAPAPAPA(SEQ ID NO: 20)-eGFP chimera DNA sequence)
SEQ ID NO: 364
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCACCTGCGATGGTTAGCAAAGGTGAAGAACTGTTTACCGGCGTTGTGCCGATTCTGGTGGAACTGGATGG
TGATGTGAATGGCCATAAATTTAGCGTTagcGGCGAAGGCGAAGGTGATGCGACCtatGGTAAACTGACCCTGAAAT
TTATTTGCACCACCGGTAAACTGCCGGTTCCGTGGCCGACCCTGGTGACCACCCTGACCTATGGCGTTCAGTGCTTT
AGCCGCTATCCGGATCATATGAAAcagCATGATTTCTTTAAAAGCGCGATGCCGGAAGGCTATGTGCAGGAACGTAC
CATTtttTTCAAAGATGATGGCaacTATAAAACCCGTGCGGAAGTTAAATTTGAAGGCGATACCCTGGTGAACCGCA
TTGAACTGAAAGGTATTGATTTTAAAGAAGATGGCAACATTCTGGGTCATAAACTGGAATATAATtacAACAGCCAT
AATGTGTATATTatgGCCGATAAACAGAAAAATGGCATCAAAgtgAACTTTAAAATCCGTCACAACattGAAGATGG
TAGCGTGCAGCTGGCGGATCATTATCAGCAGAATACCCCGATTGGTGATGGCCCGGTGCTGCTGCCGGATAATCATT
ATCTGAGCACCCAGAGCgcgCTGAGCAAAGATCCGAATGAAAAACGTGATCATATGGTGCTGCTGGAATTTGTTACC
GCCGCGGGCATTACCctgGGTATGGATGAACTGTATAAA
(Cas9-APAPAPAPAPAPAPAPAPAPAPAPA(SEQ ID NO: 21)-eGFP chimera DNA sequence)
SEQ ID NO: 365
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCTCCTGCACCTGCGATGGTTAGCAAAGGTGAAGAACTGTTTACCGGCGTTGTGCCGATTCTGGTGGAACT
GGATGGTGATGTGAATGGCCATAAATTTAGCGTTagcGGCGAAGGCGAAGGTGATGCGACCtatGGTAAACTGACCC
TGAAATTTATTTGCACCACCGGTAAACTGCCGGTTCCGTGGCCGACCCTGGTGACCACCCTGACCTATGGCGTTCAG
TGCTTTAGCCGCTATCCGGATCATATGAAAcagCATGATTTCTTTAAAAGCGCGATGCCGGAAGGCTATGTGCAGGA
ACGTACCATTtttTTCAAAGATGATGGCaacTATAAAACCCGTGCGGAAGTTAAATTTGAAGGCGATACCCTGGTGA
ACCGCATTGAACTGAAAGGTATTGATTTTAAAGAAGATGGCAACATTCTGGGTCATAAACTGGAATATAATtacAAC
AGCCATAATGTGTATATTatgGCCGATAAACAGAAAAATGGCATCAAAgtgAACTTTAAAATCCGTCACAACattGA
AGATGGTAGCGTGCAGCTGGCGGATCATTATCAGCAGAATACCCCGATTGGTGATGGCCCGGTGCTGCTGCCGGATA
ATCATTATCTGAGCACCCAGAGCgcgCTGAGCAAAGATCCGAATGAAAAACGTGATCATATGGTGCTGCTGGAATTT
GTTACCGCCGCGGGCATTACCctgGGTATGGATGAACTGTATAAA
(Cas9-APAPAPAPAPAPAPAPAPAPAPAPAPA(SEQ ID NO: 22)-eGFP chimera DNA sequence)
SEQ ID NO: 366
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCGCCAGCTCCTGCACCTGCGATGGTTAGCAAAGGTGAAGAACTGTTTACCGGCGTTGTGCCGATTCTGGT
GGAACTGGATGGTGATGTGAATGGCCATAAATTTAGCGTTagcGGCGAAGGCGAAGGTGATGCGACCtatGGTAAAC
TGACCCTGAAATTTATTTGCACCACCGGTAAACTGCCGGTTCCGTGGCCGACCCTGGTGACCACCCTGACCTATGGC
GTTCAGTGCTTTAGCCGCTATCCGGATCATATGAAAcagCATGATTTCTTTAAAAGCGCGATGCCGGAAGGCTATGT
GCAGGAACGTACCATTtttTTCAAAGATGATGGCaacTATAAAACCCGTGCGGAAGTTAAATTTGAAGGCGATACCC
TGGTGAACCGCATTGAACTGAAAGGTATTGATTTTAAAGAAGATGGCAACATTCTGGGTCATAAACTGGAATATAAT
tacAACAGCCATAATGTGTATATTatgGCCGATAAACAGAAAAATGGCATCAAAgtgAACTTTAAAATCCGTCACAA
CattGAAGATGGTAGCGTGCAGCTGGCGGATCATTATCAGCAGAATACCCCGATTGGTGATGGCCCGGTGCTGCTGC
CGGATAATCATTATCTGAGCACCCAGAGCgcgCTGAGCAAAGATCCGAATGAAAAACGTGATCATATGGTGCTGCTG
GAATTTGTTACCGCCGCGGGCATTACCctgGGTATGGATGAACTGTATAAA
(Cas9-APAPAPAPAPAPAPAPAPAPAPAPAPAPA(SEQ ID NO: 23)-eGFP chimera DNA sequence)
SEQ ID NO: 367
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCGCCAGCTCCTGCACCTGCTCCAGCGATGGTTAGCAAAGGTGAAGAACTGTTTACCGGCGTTGTGCCGAT
TCTGGTGGAACTGGATGGTGATGTGAATGGCCATAAATTTAGCGTTagcGGCGAAGGCGAAGGTGATGCGACCtatG
GTAAACTGACCCTGAAATTTATTTGCACCACCGGTAAACTGCCGGTTCCGTGGCCGACCCTGGTGACCACCCTGACC
TATGGCGTTCAGTGCTTTAGCCGCTATCCGGATCATATGAAAcagCATGATTTCTTTAAAAGCGCGATGCCGGAAGG
CTATGTGCAGGAACGTACCATTtttTTCAAAGATGATGGCaacTATAAAACCCGTGCGGAAGTTAAATTTGAAGGCG
ATACCCTGGTGAACCGCATTGAACTGAAAGGTATTGATTTTAAAGAAGATGGCAACATTCTGGGTCATAAACTGGAA
TATAATtacAACAGCCATAATGTGTATATTatgGCCGATAAACAGAAAAATGGCATCAAAgtgAACTTTAAAATCCG
TCACAACattGAAGATGGTAGCGTGCAGCTGGCGGATCATTATCAGCAGAATACCCCGATTGGTGATGGCCCGGTGC
TGCTGCCGGATAATCATTATCTGAGCACCCAGAGCgcgCTGAGCAAAGATCCGAATGAAAAACGTGATCATATGGTG
CTGCTGGAATTTGTTACCGCCGCGGGCATTACCctgGGTATGGATGAACTGTATAAA
(Cas9-GSAGSAAGSGEF(SEQ ID NO: 6)-mCherry chimera DNA sequence)
SEQ ID NO: 368
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGTAGCGCAGGTAGTGCAGCAGGTAGCGGTGAATTTATGGTGAGCAAAGGTGAA
GAGGATAATATGGCCATCATCAAAGAATTCATGCGCTTCAAAGTTCATATGGAAGGTAGCGTTAATGGCCACGAATT
TGAAATTGAAGGTGAAGGCGAAGGTCGTCCGTATGAAGGCACCCAGACCGCAAAACTGAAAGTTACCAAAGGTGGTC
CGCTGCCGTTTGCATGGGATATTCTGAGTCCGCAGTTTATGTATGGTAGCAAAGCCTATGTTAAACATCCGGCAGAT
ATCCCGGATTATCTGAAACTGAGCTTTCCGGAAGGTTTTAAATGGGAACGTGTGATGAATTTTGAAGATGGTGGTGT
TGTTACCGTTACACAGGATAGCAGCCTGCAGGATGGTGAATTTATCTATAAAGTTAAACTGCGTGGCACGAATTTTC
CGAGTGATGGTCCGGTTATGCAGAAAAAAACCATGGGTTGGGAAGCAAGCAGCGAACGTATGTATCCGGAAGATGGC
GCACTGAAAGGTGAAATTAAACAGCGTCTGAAGCTGAAAGATGGCGGTCATTATGATGCAGAAGTTAAAACCACCTA
CAAAGCCAAAAAACCGGTTCAGCTGCCTGGTGCATATAACGTTAACATTAAACTGGATATCACCAGCCACAACGAGG
ATTATACCATTGTTGAACAGTATGAACGTGCAGAAGGTCGCCATAGTACCGGTGGTATGGATGAACTGTATAAA
(Cas9-(GGGGS)₄-(SEQ ID NO: 7)mCherry chimera DNA sequence)
SEQ ID NO: 369
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGAGGCGGAGGATCAGGCGGTGGCGGAAGTGGTGGAGGTGGGTCGGGAGGTGGT
GGTAGCATGGTGAGCAAAGGTGAAGAGGATAATATGGCCATCATCAAAGAATTCATGCGCTTCAAAGTTCATATGGA
AGGTAGCGTTAATGGCCACGAATTTGAAATTGAAGGTGAAGGCGAAGGTCGTCCGTATGAAGGCACCCAGACCGCAA
AACTGAAAGTTACCAAAGGTGGTCCGCTGCCGTTTGCATGGGATATTCTGAGTCCGCAGTTTATGTATGGTAGCAAA
GCCTATGTTAAACATCCGGCAGATATCCCGGATTATCTGAAACTGAGCTTTCCGGAAGGTTTTAAATGGGAACGTGT
GATGAATTTTGAAGATGGTGGTGTTGTTACCGTTACACAGGATAGCAGCCTGCAGGATGGTGAATTTATCTATAAAG
TTAAACTGCGTGGCACGAATTTTCCGAGTGATGGTCCGGTTATGCAGAAAAAAACCATGGGTTGGGAAGCAAGCAGC
GAACGTATGTATCCGGAAGATGGCGCACTGAAAGGTGAAATTAAACAGCGTCTGAAGCTGAAAGATGGCGGTCATTA
TGATGCAGAAGTTAAAACCACCTACAAAGCCAAAAAACCGGTTCAGCTGCCTGGTGCATATAACGTTAACATTAAAC
TGGATATCACCAGCCACAACGAGGATTATACCATTGTTGAACAGTATGAACGTGCAGAAGGTCGCCATAGTACCGGT
GGTATGGATGAACTGTATAAA
(Cas9-GGGGSEAAAKGGGGS(SEQ ID NO: 37)-mCherry chimera DNA sequence)
SEQ ID NO: 370
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGGAGGCGGAGGGTCGGAAGCGGCAGCAAAAGGAGGCGGAGGAAGTATGGTGAGC
AAAGGTGAAGAGGATAATATGGCCATCATCAAAGAATTCATGCGCTTCAAAGTTCATATGGAAGGTAGCGTTAATGG
CCACGAATTTGAAATTGAAGGTGAAGGCGAAGGTCGTCCGTATGAAGGCACCCAGACCGCAAAACTGAAAGTTACCA
AAGGTGGTCCGCTGCCGTTTGCATGGGATATTCTGAGTCCGCAGTTTATGTATGGTAGCAAAGCCTATGTTAAACAT
CCGGCAGATATCCCGGATTATCTGAAACTGAGCTTTCCGGAAGGTTTTAAATGGGAACGTGTGATGAATTTTGAAGA
TGGTGGTGTTGTTACCGTTACACAGGATAGCAGCCTGCAGGATGGTGAATTTATCTATAAAGTTAAACTGCGTGGCA
CGAATTTTCCGAGTGATGGTCCGGTTATGCAGAAAAAAACCATGGGTTGGGAAGCAAGCAGCGAACGTATGTATCCG
GAAGATGGCGCACTGAAAGGTGAAATTAAACAGCGTCTGAAGCTGAAAGATGGCGGTCATTATGATGCAGAAGTTAA
AACCACCTACAAAGCCAAAAAACCGGTTCAGCTGCCTGGTGCATATAACGTTAACATTAAACTGGATATCACCAGCC
ACAACGAGGATTATACCATTGTTGAACAGTATGAACGTGCAGAAGGTCGCCATAGTACCGGTGGTATGGATGAACTG
TATAAA
(Cas9-A(EAAAK)₄A(SEQ ID NO: 8)-mCherry chimera DNA sequence)
SEQ ID NO: 371
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTGAGGCCGCAGCAAAAGAGGCTGCCGCTAAGGAGGCTGCTGCAAAAGAAGCA
GCAGCGAAAGCTATGGTGAGCAAAGGTGAAGAGGATAATATGGCCATCATCAAAGAATTCATGCGCTTCAAAGTTCA
TATGGAAGGTAGCGTTAATGGCCACGAATTTGAAATTGAAGGTGAAGGCGAAGGTCGTCCGTATGAAGGCACCCAGA
CCGCAAAACTGAAAGTTACCAAAGGTGGTCCGCTGCCGTTTGCATGGGATATTCTGAGTCCGCAGTTTATGTATGGT
AGCAAAGCCTATGTTAAACATCCGGCAGATATCCCGGATTATCTGAAACTGAGCTTTCCGGAAGGTTTTAAATGGGA
ACGTGTGATGAATTTTGAAGATGGTGGTGTTGTTACCGTTACACAGGATAGCAGCCTGCAGGATGGTGAATTTATCT
ATAAAGTTAAACTGCGTGGCACGAATTTTCCGAGTGATGGTCCGGTTATGCAGAAAAAAACCATGGGTTGGGAAGCA
AGCAGCGAACGTATGTATCCGGAAGATGGCGCACTGAAAGGTGAAATTAAACAGCGTCTGAAGCTGAAAGATGGCGG
TCATTATGATGCAGAAGTTAAAACCACCTACAAAGCCAAAAAACCGGTTCAGCTGCCTGGTGCATATAACGTTAACA
TTAAACTGGATATCACCAGCCACAACGAGGATTATACCATTGTTGAACAGTATGAACGTGCAGAAGGTCGCCATAGT
ACCGGTGGTATGGATGAACTGTATAAA
(Cas9-A(EAAAK)₄ALEA(EAAAK)₄A(SEQ ID NO: 9)-mCherry chimera DNA sequence)
SEQ ID NO: 372
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCCGAAGCAGCAGCCAAAGAAGCTGCCGCAAAGGAGGCGGCAGCTAAAGAAGCG
GCCGCTAAGGCATTGGAAGCGGAGGCGGCTGCCAAGGAGGCTGCGGCGAAAGAAGCTGCGGCCAAAGAAGCGGCTGC
AAAAGCGATGGTGAGCAAAGGTGAAGAGGATAATATGGCCATCATCAAAGAATTCATGCGCTTCAAAGTTCATATGG
AAGGTAGCGTTAATGGCCACGAATTTGAAATTGAAGGTGAAGGCGAAGGTCGTCCGTATGAAGGCACCCAGACCGCA
AAACTGAAAGTTACCAAAGGTGGTCCGCTGCCGTTTGCATGGGATATTCTGAGTCCGCAGTTTATGTATGGTAGCAA
AGCCTATGTTAAACATCCGGCAGATATCCCGGATTATCTGAAACTGAGCTTTCCGGAAGGTTTTAAATGGGAACGTG
TGATGAATTTTGAAGATGGTGGTGTTGTTACCGTTACACAGGATAGCAGCCTGCAGGATGGTGAATTTATCTATAAA
GTTAAACTGCGTGGCACGAATTTTCCGAGTGATGGTCCGGTTATGCAGAAAAAAACCATGGGTTGGGAAGCAAGCAG
CGAACGTATGTATCCGGAAGATGGCGCACTGAAAGGTGAAATTAAACAGCGTCTGAAGCTGAAAGATGGCGGTCATT
ATGATGCAGAAGTTAAAACCACCTACAAAGCCAAAAAACCGGTTCAGCTGCCTGGTGCATATAACGTTAACATTAAA
CTGGATATCACCAGCCACAACGAGGATTATACCATTGTTGAACAGTATGAACGTGCAGAAGGTCGCCATAGTACCGG
TGGTATGGATGAACTGTATAAA
(Cas9-LEA(EAAAK)₄ALEA(EAAAK)₄ALE(SEQ ID NO: 10)-mCherry chimera DNA sequence)
SEQ ID NO: 373
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATTTAGAGGCTGAGGCTGCTGCCAAGGAGGCTGCTGCGAAGGAAGCTGCAGCTAAA
GAGGCAGCGGCCAAAGCACTTGAGGCCGAAGCCGCCGCGAAAGAAGCCGCTGCTAAGGAGGCAGCCGCCAAAGAAGC
GGCAGCTAAAGCATTAGAAATGGTGAGCAAAGGTGAAGAGGATAATATGGCCATCATCAAAGAATTCATGCGCTTCA
AAGTTCATATGGAAGGTAGCGTTAATGGCCACGAATTTGAAATTGAAGGTGAAGGCGAAGGTCGTCCGTATGAAGGC
ACCCAGACCGCAAAACTGAAAGTTACCAAAGGTGGTCCGCTGCCGTTTGCATGGGATATTCTGAGTCCGCAGTTTAT
GTATGGTAGCAAAGCCTATGTTAAACATCCGGCAGATATCCCGGATTATCTGAAACTGAGCTTTCCGGAAGGTTTTA
AATGGGAACGTGTGATGAATTTTGAAGATGGTGGTGTTGTTACCGTTACACAGGATAGCAGCCTGCAGGATGGTGAA
TTTATCTATAAAGTTAAACTGCGTGGCACGAATTTTCCGAGTGATGGTCCGGTTATGCAGAAAAAAACCATGGGTTG
GGAAGCAAGCAGCGAACGTATGTATCCGGAAGATGGCGCACTGAAAGGTGAAATTAAACAGCGTCTGAAGCTGAAAG
ATGGCGGTCATTATGATGCAGAAGTTAAAACCACCTACAAAGCCAAAAAACCGGTTCAGCTGCCTGGTGCATATAAC
GTTAACATTAAACTGGATATCACCAGCCACAACGAGGATTATACCATTGTTGAACAGTATGAACGTGCAGAAGGTCG
CCATAGTACCGGTGGTATGGATGAACTGTATAAA
(Cas9-APA-mCherry chimera DNA sequence)
SEQ ID NO: 374
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCACCTGCGATGGTGAGCAAAGGTGAAGAGGATAATATGGCCATCATCAAAGAA
TTCATGCGCTTCAAAGTTCATATGGAAGGTAGCGTTAATGGCCACGAATTTGAAATTGAAGGTGAAGGCGAAGGTCG
TCCGTATGAAGGCACCCAGACCGCAAAACTGAAAGTTACCAAAGGTGGTCCGCTGCCGTTTGCATGGGATATTCTGA
GTCCGCAGTTTATGTATGGTAGCAAAGCCTATGTTAAACATCCGGCAGATATCCCGGATTATCTGAAACTGAGCTTT
CCGGAAGGTTTTAAATGGGAACGTGTGATGAATTTTGAAGATGGTGGTGTTGTTACCGTTACACAGGATAGCAGCCT
GCAGGATGGTGAATTTATCTATAAAGTTAAACTGCGTGGCACGAATTTTCCGAGTGATGGTCCGGTTATGCAGAAAA
AAACCATGGGTTGGGAAGCAAGCAGCGAACGTATGTATCCGGAAGATGGCGCACTGAAAGGTGAAATTAAACAGCGT
CTGAAGCTGAAAGATGGCGGTCATTATGATGCAGAAGTTAAAACCACCTACAAAGCCAAAAAACCGGTTCAGCTGCC
TGGTGCATATAACGTTAACATTAAACTGGATATCACCAGCCACAACGAGGATTATACCATTGTTGAACAGTATGAAC
GTGCAGAAGGTCGCCATAGTACCGGTGGTATGGATGAACTGTATAAA
(Cas9-APAPA(SEQ ID NO: 11)-mCherry chimera DNA sequence)
SEQ ID NO: 375
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTCCTGCACCTGCGATGGTGAGCAAAGGTGAAGAGGATAATATGGCCATCATC
AAAGAATTCATGCGCTTCAAAGTTCATATGGAAGGTAGCGTTAATGGCCACGAATTTGAAATTGAAGGTGAAGGCGA
AGGTCGTCCGTATGAAGGCACCCAGACCGCAAAACTGAAAGTTACCAAAGGTGGTCCGCTGCCGTTTGCATGGGATA
TTCTGAGTCCGCAGTTTATGTATGGTAGCAAAGCCTATGTTAAACATCCGGCAGATATCCCGGATTATCTGAAACTG
AGCTTTCCGGAAGGTTTTAAATGGGAACGTGTGATGAATTTTGAAGATGGTGGTGTTGTTACCGTTACACAGGATAG
CAGCCTGCAGGATGGTGAATTTATCTATAAAGTTAAACTGCGTGGCACGAATTTTCCGAGTGATGGTCCGGTTATGC
AGAAAAAAACCATGGGTTGGGAAGCAAGCAGCGAACGTATGTATCCGGAAGATGGCGCACTGAAAGGTGAAATTAAA
CAGCGTCTGAAGCTGAAAGATGGCGGTCATTATGATGCAGAAGTTAAAACCACCTACAAAGCCAAAAAACCGGTTCA
GCTGCCTGGTGCATATAACGTTAACATTAAACTGGATATCACCAGCCACAACGAGGATTATACCATTGTTGAACAGT
ATGAACGTGCAGAAGGTCGCCATAGTACCGGTGGTATGGATGAACTGTATAAA
(Cas9-APAPAPA(SEQ ID NO: 12)-mCherry chimera DNA sequence)
SEQ ID NO: 376
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCTCCTGCACCTGCGATGGTGAGCAAAGGTGAAGAGGATAATATGGCC
ATCATCAAAGAATTCATGCGCTTCAAAGTTCATATGGAAGGTAGCGTTAATGGCCACGAATTTGAAATTGAAGGTGA
AGGCGAAGGTCGTCCGTATGAAGGCACCCAGACCGCAAAACTGAAAGTTACCAAAGGTGGTCCGCTGCCGTTTGCAT
GGGATATTCTGAGTCCGCAGTTTATGTATGGTAGCAAAGCCTATGTTAAACATCCGGCAGATATCCCGGATTATCTG
AAACTGAGCTTTCCGGAAGGTTTTAAATGGGAACGTGTGATGAATTTTGAAGATGGTGGTGTTGTTACCGTTACACA
GGATAGCAGCCTGCAGGATGGTGAATTTATCTATAAAGTTAAACTGCGTGGCACGAATTTTCCGAGTGATGGTCCGG
TTATGCAGAAAAAAACCATGGGTTGGGAAGCAAGCAGCGAACGTATGTATCCGGAAGATGGCGCACTGAAAGGTGAA
ATTAAACAGCGTCTGAAGCTGAAAGATGGCGGTCATTATGATGCAGAAGTTAAAACCACCTACAAAGCCAAAAAACC
GGTTCAGCTGCCTGGTGCATATAACGTTAACATTAAACTGGATATCACCAGCCACAACGAGGATTATACCATTGTTG
AACAGTATGAACGTGCAGAAGGTCGCCATAGTACCGGTGGTATGGATGAACTGTATAAA
(Cas9-APAPAPAPA(SEQ ID NO: 13)-mCherry chimera DNA sequence)
SEQ ID NO: 377
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCTCCTGCGCCAGCTCCTGCACCTGCGATGGTGAGCAAAGGTGAAGAGGATAAT
ATGGCCATCATCAAAGAATTCATGCGCTTCAAAGTTCATATGGAAGGTAGCGTTAATGGCCACGAATTTGAAATTGA
AGGTGAAGGCGAAGGTCGTCCGTATGAAGGCACCCAGACCGCAAAACTGAAAGTTACCAAAGGTGGTCCGCTGCCGT
TTGCATGGGATATTCTGAGTCCGCAGTTTATGTATGGTAGCAAAGCCTATGTTAAACATCCGGCAGATATCCCGGAT
TATCTGAAACTGAGCTTTCCGGAAGGTTTTAAATGGGAACGTGTGATGAATTTTGAAGATGGTGGTGTTGTTACCGT
TACACAGGATAGCAGCCTGCAGGATGGTGAATTTATCTATAAAGTTAAACTGCGTGGCACGAATTTTCCGAGTGATG
GTCCGGTTATGCAGAAAAAAACCATGGGTTGGGAAGCAAGCAGCGAACGTATGTATCCGGAAGATGGCGCACTGAAA
GGTGAAATTAAACAGCGTCTGAAGCTGAAAGATGGCGGTCATTATGATGCAGAAGTTAAAACCACCTACAAAGCCAA
AAAACCGGTTCAGCTGCCTGGTGCATATAACGTTAACATTAAACTGGATATCACCAGCCACAACGAGGATTATACCA
TTGTTGAACAGTATGAACGTGCAGAAGGTCGCCATAGTACCGGTGGTATGGATGAACTGTATAAA
(Cas9-APAPAPAPAPA(SEQ ID NO: 14)-mCherry chimera DNA sequence)
SEQ ID NO: 378
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCCGCTCCTGCGCCAGCTCCTGCACCTGCGATGGTGAGCAAAGGTGAAGAG
GATAATATGGCCATCATCAAAGAATTCATGCGCTTCAAAGTTCATATGGAAGGTAGCGTTAATGGCCACGAATTTGA
AATTGAAGGTGAAGGCGAAGGTCGTCCGTATGAAGGCACCCAGACCGCAAAACTGAAAGTTACCAAAGGTGGTCCGC
TGCCGTTTGCATGGGATATTCTGAGTCCGCAGTTTATGTATGGTAGCAAAGCCTATGTTAAACATCCGGCAGATATC
CCGGATTATCTGAAACTGAGCTTTCCGGAAGGTTTTAAATGGGAACGTGTGATGAATTTTGAAGATGGTGGTGTTGT
TACCGTTACACAGGATAGCAGCCTGCAGGATGGTGAATTTATCTATAAAGTTAAACTGCGTGGCACGAATTTTCCGA
GTGATGGTCCGGTTATGCAGAAAAAAACCATGGGTTGGGAAGCAAGCAGCGAACGTATGTATCCGGAAGATGGCGCA
CTGAAAGGTGAAATTAAACAGCGTCTGAAGCTGAAAGATGGCGGTCATTATGATGCAGAAGTTAAAACCACCTACAA
AGCCAAAAAACCGGTTCAGCTGCCTGGTGCATATAACGTTAACATTAAACTGGATATCACCAGCCACAACGAGGATT
ATACCATTGTTGAACAGTATGAACGTGCAGAAGGTCGCCATAGTACCGGTGGTATGGATGAACTGTATAAA
(Cas9-APAPAPAPAPAPA(SEQ ID NO: 15)-mCherry chimera DNA sequence)
SEQ ID NO: 379
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCACCTGCGATGGTGAGCAAAGGT
GAAGAGGATAATATGGCCATCATCAAAGAATTCATGCGCTTCAAAGTTCATATGGAAGGTAGCGTTAATGGCCACGA
ATTTGAAATTGAAGGTGAAGGCGAAGGTCGTCCGTATGAAGGCACCCAGACCGCAAAACTGAAAGTTACCAAAGGTG
GTCCGCTGCCGTTTGCATGGGATATTCTGAGTCCGCAGTTTATGTATGGTAGCAAAGCCTATGTTAAACATCCGGCA
GATATCCCGGATTATCTGAAACTGAGCTTTCCGGAAGGTTTTAAATGGGAACGTGTGATGAATTTTGAAGATGGTGG
TGTTGTTACCGTTACACAGGATAGCAGCCTGCAGGATGGTGAATTTATCTATAAAGTTAAACTGCGTGGCACGAATT
TTCCGAGTGATGGTCCGGTTATGCAGAAAAAAACCATGGGTTGGGAAGCAAGCAGCGAACGTATGTATCCGGAAGAT
GGCGCACTGAAAGGTGAAATTAAACAGCGTCTGAAGCTGAAAGATGGCGGTCATTATGATGCAGAAGTTAAAACCAC
CTACAAAGCCAAAAAACCGGTTCAGCTGCCTGGTGCATATAACGTTAACATTAAACTGGATATCACCAGCCACAACG
AGGATTATACCATTGTTGAACAGTATGAACGTGCAGAAGGTCGCCATAGTACCGGTGGTATGGATGAACTGTATAAA
(Cas9-APAPAPAPAPAPAPA(SEQ ID NO: 16)-mCherry chimera DNA sequence)
SEQ ID NO: 380
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCGATGGTGAGC
AAAGGTGAAGAGGATAATATGGCCATCATCAAAGAATTCATGCGCTTCAAAGTTCATATGGAAGGTAGCGTTAATGG
CCACGAATTTGAAATTGAAGGTGAAGGCGAAGGTCGTCCGTATGAAGGCACCCAGACCGCAAAACTGAAAGTTACCA
AAGGTGGTCCGCTGCCGTTTGCATGGGATATTCTGAGTCCGCAGTTTATGTATGGTAGCAAAGCCTATGTTAAACAT
CCGGCAGATATCCCGGATTATCTGAAACTGAGCTTTCCGGAAGGTTTTAAATGGGAACGTGTGATGAATTTTGAAGA
TGGTGGTGTTGTTACCGTTACACAGGATAGCAGCCTGCAGGATGGTGAATTTATCTATAAAGTTAAACTGCGTGGCA
CGAATTTTCCGAGTGATGGTCCGGTTATGCAGAAAAAAACCATGGGTTGGGAAGCAAGCAGCGAACGTATGTATCCG
GAAGATGGCGCACTGAAAGGTGAAATTAAACAGCGTCTGAAGCTGAAAGATGGCGGTCATTATGATGCAGAAGTTAA
AACCACCTACAAAGCCAAAAAACCGGTTCAGCTGCCTGGTGCATATAACGTTAACATTAAACTGGATATCACCAGCC
ACAACGAGGATTATACCATTGTTGAACAGTATGAACGTGCAGAAGGTCGCCATAGTACCGGTGGTATGGATGAACTG
TATAAA
(Cas9-APAPAPAPAPAPAPAPA(SEQ ID NO: 17)-mCherry chimera DNA sequence)
SEQ ID NO: 381
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCGCCAGCTCCTGCACCTGCGATG
GTGAGCAAAGGTGAAGAGGATAATATGGCCATCATCAAAGAATTCATGCGCTTCAAAGTTCATATGGAAGGTAGCGT
TAATGGCCACGAATTTGAAATTGAAGGTGAAGGCGAAGGTCGTCCGTATGAAGGCACCCAGACCGCAAAACTGAAAG
TTACCAAAGGTGGTCCGCTGCCGTTTGCATGGGATATTCTGAGTCCGCAGTTTATGTATGGTAGCAAAGCCTATGTT
AAACATCCGGCAGATATCCCGGATTATCTGAAACTGAGCTTTCCGGAAGGTTTTAAATGGGAACGTGTGATGAATTT
TGAAGATGGTGGTGTTGTTACCGTTACACAGGATAGCAGCCTGCAGGATGGTGAATTTATCTATAAAGTTAAACTGC
GTGGCACGAATTTTCCGAGTGATGGTCCGGTTATGCAGAAAAAAACCATGGGTTGGGAAGCAAGCAGCGAACGTATG
TATCCGGAAGATGGCGCACTGAAAGGTGAAATTAAACAGCGTCTGAAGCTGAAAGATGGCGGTCATTATGATGCAGA
AGTTAAAACCACCTACAAAGCCAAAAAACCGGTTCAGCTGCCTGGTGCATATAACGTTAACATTAAACTGGATATCA
CCAGCCACAACGAGGATTATACCATTGTTGAACAGTATGAACGTGCAGAAGGTCGCCATAGTACCGGTGGTATGGAT
GAACTGTATAAA
(Cas9-APAPAPAPAPAPAPAPAPA(SEQ ID NO: 18)-mCherry chimera DNA sequence)
SEQ ID NO: 382
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCGCCAGCTCCTGCACCT
GCGATGGTGAGCAAAGGTGAAGAGGATAATATGGCCATCATCAAAGAATTCATGCGCTTCAAAGTTCATATGGAAGG
TAGCGTTAATGGCCACGAATTTGAAATTGAAGGTGAAGGCGAAGGTCGTCCGTATGAAGGCACCCAGACCGCAAAAC
TGAAAGTTACCAAAGGTGGTCCGCTGCCGTTTGCATGGGATATTCTGAGTCCGCAGTTTATGTATGGTAGCAAAGCC
TATGTTAAACATCCGGCAGATATCCCGGATTATCTGAAACTGAGCTTTCCGGAAGGTTTTAAATGGGAACGTGTGAT
GAATTTTGAAGATGGTGGTGTTGTTACCGTTACACAGGATAGCAGCCTGCAGGATGGTGAATTTATCTATAAAGTTA
AACTGCGTGGCACGAATTTTCCGAGTGATGGTCCGGTTATGCAGAAAAAAACCATGGGTTGGGAAGCAAGCAGCGAA
CGTATGTATCCGGAAGATGGCGCACTGAAAGGTGAAATTAAACAGCGTCTGAAGCTGAAAGATGGCGGTCATTATGA
TGCAGAAGTTAAAACCACCTACAAAGCCAAAAAACCGGTTCAGCTGCCTGGTGCATATAACGTTAACATTAAACTGG
ATATCACCAGCCACAACGAGGATTATACCATTGTTGAACAGTATGAACGTGCAGAAGGTCGCCATAGTACCGGTGGT
ATGGATGAACTGTATAAA
(Cas9-APAPAPAPAPAPAPAPAPAPA(SEQ ID NO: 19)-mCherry chimera DNA sequence)
SEQ ID NO: 383
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCTCCTGCGCCAGCTCCT
GCACCTGCGATGGTGAGCAAAGGTGAAGAGGATAATATGGCCATCATCAAAGAATTCATGCGCTTCAAAGTTCATAT
GGAAGGTAGCGTTAATGGCCACGAATTTGAAATTGAAGGTGAAGGCGAAGGTCGTCCGTATGAAGGCACCCAGACCG
CAAAACTGAAAGTTACCAAAGGTGGTCCGCTGCCGTTTGCATGGGATATTCTGAGTCCGCAGTTTATGTATGGTAGC
AAAGCCTATGTTAAACATCCGGCAGATATCCCGGATTATCTGAAACTGAGCTTTCCGGAAGGTTTTAAATGGGAACG
TGTGATGAATTTTGAAGATGGTGGTGTTGTTACCGTTACACAGGATAGCAGCCTGCAGGATGGTGAATTTATCTATA
AAGTTAAACTGCGTGGCACGAATTTTCCGAGTGATGGTCCGGTTATGCAGAAAAAAACCATGGGTTGGGAAGCAAGC
AGCGAACGTATGTATCCGGAAGATGGCGCACTGAAAGGTGAAATTAAACAGCGTCTGAAGCTGAAAGATGGCGGTCA
TTATGATGCAGAAGTTAAAACCACCTACAAAGCCAAAAAACCGGTTCAGCTGCCTGGTGCATATAACGTTAACATTA
AACTGGATATCACCAGCCACAACGAGGATTATACCATTGTTGAACAGTATGAACGTGCAGAAGGTCGCCATAGTACC
GGTGGTATGGATGAACTGTATAAA
(Cas9-APAPAPAPAPAPAPAPAPAPAPA(SEQ ID NO: 20)-mCherry chimera DNA sequence)
SEQ ID NO: 384
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCACCTGCGATGGTGAGCAAAGGTGAAGAGGATAATATGGCCATCATCAAAGAATTCATGCGCTTCAAAGT
TCATATGGAAGGTAGCGTTAATGGCCACGAATTTGAAATTGAAGGTGAAGGCGAAGGTCGTCCGTATGAAGGCACCC
AGACCGCAAAACTGAAAGTTACCAAAGGTGGTCCGCTGCCGTTTGCATGGGATATTCTGAGTCCGCAGTTTATGTAT
GGTAGCAAAGCCTATGTTAAACATCCGGCAGATATCCCGGATTATCTGAAACTGAGCTTTCCGGAAGGTTTTAAATG
GGAACGTGTGATGAATTTTGAAGATGGTGGTGTTGTTACCGTTACACAGGATAGCAGCCTGCAGGATGGTGAATTTA
TCTATAAAGTTAAACTGCGTGGCACGAATTTTCCGAGTGATGGTCCGGTTATGCAGAAAAAAACCATGGGTTGGGAA
GCAAGCAGCGAACGTATGTATCCGGAAGATGGCGCACTGAAAGGTGAAATTAAACAGCGTCTGAAGCTGAAAGATGG
CGGTCATTATGATGCAGAAGTTAAAACCACCTACAAAGCCAAAAAACCGGTTCAGCTGCCTGGTGCATATAACGTTA
ACATTAAACTGGATATCACCAGCCACAACGAGGATTATACCATTGTTGAACAGTATGAACGTGCAGAAGGTCGCCAT
AGTACCGGTGGTATGGATGAACTGTATAAA
(Cas9-APAPAPAPAPAPAPAPAPAPAPAPA(SEQ ID NO: 21)-mCherry chimera DNA sequence)
SEQ ID NO: 385
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCTCCTGCACCTGCGATGGTGAGCAAAGGTGAAGAGGATAATATGGCCATCATCAAAGAATTCATGCGCTT
CAAAGTTCATATGGAAGGTAGCGTTAATGGCCACGAATTTGAAATTGAAGGTGAAGGCGAAGGTCGTCCGTATGAAG
GCACCCAGACCGCAAAACTGAAAGTTACCAAAGGTGGTCCGCTGCCGTTTGCATGGGATATTCTGAGTCCGCAGTTT
ATGTATGGTAGCAAAGCCTATGTTAAACATCCGGCAGATATCCCGGATTATCTGAAACTGAGCTTTCCGGAAGGTTT
TAAATGGGAACGTGTGATGAATTTTGAAGATGGTGGTGTTGTTACCGTTACACAGGATAGCAGCCTGCAGGATGGTG
AATTTATCTATAAAGTTAAACTGCGTGGCACGAATTTTCCGAGTGATGGTCCGGTTATGCAGAAAAAAACCATGGGT
TGGGAAGCAAGCAGCGAACGTATGTATCCGGAAGATGGCGCACTGAAAGGTGAAATTAAACAGCGTCTGAAGCTGAA
AGATGGCGGTCATTATGATGCAGAAGTTAAAACCACCTACAAAGCCAAAAAACCGGTTCAGCTGCCTGGTGCATATA
ACGTTAACATTAAACTGGATATCACCAGCCACAACGAGGATTATACCATTGTTGAACAGTATGAACGTGCAGAAGGT
CGCCATAGTACCGGTGGTATGGATGAACTGTATAAA
(Cas9-APAPAPAPAPAPAPAPAPAPAPAPAPA(SEQ ID NO: 22)-mCherry chimera DNA sequence)
SEQ ID NO: 386
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCGCCAGCTCCTGCACCTGCGATGGTGAGCAAAGGTGAAGAGGATAATATGGCCATCATCAAAGAATTCAT
GCGCTTCAAAGTTCATATGGAAGGTAGCGTTAATGGCCACGAATTTGAAATTGAAGGTGAAGGCGAAGGTCGTCCGT
ATGAAGGCACCCAGACCGCAAAACTGAAAGTTACCAAAGGTGGTCCGCTGCCGTTTGCATGGGATATTCTGAGTCCG
CAGTTTATGTATGGTAGCAAAGCCTATGTTAAACATCCGGCAGATATCCCGGATTATCTGAAACTGAGCTTTCCGGA
AGGTTTTAAATGGGAACGTGTGATGAATTTTGAAGATGGTGGTGTTGTTACCGTTACACAGGATAGCAGCCTGCAGG
ATGGTGAATTTATCTATAAAGTTAAACTGCGTGGCACGAATTTTCCGAGTGATGGTCCGGTTATGCAGAAAAAAACC
ATGGGTTGGGAAGCAAGCAGCGAACGTATGTATCCGGAAGATGGCGCACTGAAAGGTGAAATTAAACAGCGTCTGAA
GCTGAAAGATGGCGGTCATTATGATGCAGAAGTTAAAACCACCTACAAAGCCAAAAAACCGGTTCAGCTGCCTGGTG
CATATAACGTTAACATTAAACTGGATATCACCAGCCACAACGAGGATTATACCATTGTTGAACAGTATGAACGTGCA
GAAGGTCGCCATAGTACCGGTGGTATGGATGAACTGTATAAA
(Cas9-APAPAPAPAPAPAPAPAPAPAPAPAPAPA(SEQ ID NO: 23)-
mCherry chimera DNA sequence)
SEQ ID NO: 387
ATGGACAAAAAGTACTCTATTGGCCTGGATATCGGGACCAACAGCGTCGGGTGGGCTGTTATCACCGACGAGTATAA
AGTACCTTCGAAAAAGTTCAAAGTGCTGGGCAACACCGATCGCCATTCAATCAAAAAGAACTTGATTGGTGCGCTGT
TGTTTGACTCCGGGGAAACCGCCGAGGCGACTCGCCTTAAACGTACAGCACGTCGCCGGTACACTCGGCGTAAGAAT
CGCATTTGCTATTTGCAGGAAATCTTTAGCAACGAGATGGCAAAAGTCGATGACTCGTTTTTCCACCGCCTCGAGGA
AAGCTTTCTGGTGGAGGAAGACAAAAAGCATGAGCGTCACCCGATCTTCGGCAACATTGTCGATGAAGTAGCGTATC
ATGAAAAATACCCAACCATTTACCACTTACGCAAAAAGCTGGTGGACAGCACTGACAAAGCTGATTTGCGCCTTATC
TATTTAGCCCTGGCACATATGATTAAGTTTCGTGGTCACTTCCTGATCGAAGGAGACTTAAATCCCGACAACAGTGA
TGTTGATAAATTGTTTATTCAGCTTGTCCAAACTTACAATCAACTGTTCGAGGAAAACCCGATCAATGCCTCCGGTG
TGGATGCAAAAGCCATTTTAAGTGCACGCCTTAGCAAGTCCCGTCGCTTAGAAAACCTTATCGCGCAGCTGCCCGGC
GAGAAAAAGAATGGTTTGTTTGGGAACCTTATTGCCTTGAGCTTAGGCCTCACCCCGAATTTCAAAAGTAATTTCGA
TCTTGCAGAAGACGCCAAATTACAACTGTCGAAGGATACTTATGATGACGATCTCGATAATCTGTTAGCGCAGATTG
GTGACCAATACGCCGATCTTTTTCTGGCGGCTAAAAATCTGAGCGACGCCATCTTGCTTTCGGATATTCTCCGCGTT
AACACCGAAATCACGAAAGCGCCTCTTAGTGCCAGCATGATTAAACGTTATGATGAACACCACCAGGACCTGACCTT
ACTCAAAGCGTTGGTTCGCCAGCAACTGCCAGAGAAGTACAAAGAAATCTTCTTTGATCAGTCAAAGAATGGTTATG
CCGGCTATATTGACGGGGGTGCAAGCCAAGAGGAATTCTACAAATTTATCAAGCCTATTCTGGAGAAAATGGATGGC
ACCGAAGAGTTATTGGTGAAGCTTAACCGTGAAGACCTCCTGCGGAAACAGCGCACATTCGATAATGGTTCGATCCC
ACACCAAATCCATTTGGGGGAGTTACACGCTATTTTGCGTCGCCAGGAAGACTTTTACCCTTTCCTGAAGGATAACC
GGGAGAAAATTGAGAAGATCCTTACCTTTCGTATTCCGTATTACGTAGGCCCCTTAGCACGGGGTAATAGCCGTTTC
GCGTGGATGACACGGAAGTCGGAAGAGACGATCACCCCGTGGAACTTCGAAGAGGTAGTCGACAAGGGCGCATCAGC
GCAGTCTTTTATTGAACGTATGACGAATTTCGATAAAAACTTGCCCAATGAGAAGGTGCTTCCGAAACATTCCTTGT
TATATGAATATTTTACAGTTTACAACGAGCTGACCAAGGTTAAATACGTGACGGAAGGAATGCGCAAGCCCGCTTTT
CTTAGCGGTGAGCAAAAAAAGGCGATCGTCGACCTGTTATTCAAAACGAATCGTAAGGTGACTGTAAAGCAACTCAA
AGAAGATTACTTCAAAAAGATTGAGTGCTTCGACAGCGTCGAAATCTCTGGGGTAGAGGATCGGTTTAACGCAAGTT
TAGGTACCTACCATGACCTGCTTAAAATCATTAAGGATAAAGACTTCTTAGATAATGAAGAGAACGAAGATATTCTC
GAGGACATCGTCTTGACGTTAACCTTATTTGAGGATCGTGAAATGATTGAGGAACGCCTCAAAACTTATGCCCACCT
GTTCGACGATAAGGTGATGAAGCAGCTGAAACGTCGGCGCTACACAGGATGGGGCCGCTTGAGTCGCAAACTTATTA
ACGGAATCCGTGACAAGCAATCCGGCAAAACGATTCTGGATTTCTTGAAGTCGGACGGATTTGCTAATCGCAACTTC
ATGCAGTTGATCCATGATGACTCCCTGACTTTTAAAGAGGATATTCAAAAGGCGCAGGTTAGTGGTCAAGGCGACAG
CTTACACGAACACATCGCAAATTTGGCTGGTTCGCCGGCCATTAAAAAGGGGATCCTCCAGACCGTGAAAGTTGTAG
ATGAGCTTGTTAAGGTCATGGGTCGTCATAAGCCCGAAAACATCGTGATTGAAATGGCGCGGGAGAATCAAACGACC
CAGAAAGGACAAAAGAATAGCCGTGAACGGATGAAGCGGATCGAGGAAGGCATTAAAGAGCTGGGGTCTCAAATCTT
GAAGGAACACCCTGTGGAGAACACTCAGCTCCAAAATGAAAAACTTTACCTGTACTATTTGCAGAACGGACGCGATA
TGTACGTGGACCAAGAGTTGGATATTAATCGGCTGAGTGACTACGACGTTGATCATATCGTCCCGCAGAGCTTCCTC
AAAGACGATTCTATTGACAATAAGGTACTGACGCGCTCTGATAAAAACCGTGGTAAGTCGGACAACGTGCCCTCCGA
AGAGGTTGTGAAAAAGATGAAAAATTATTGGCGCCAGCTTTTAAACGCGAAGCTGATCACACAACGTAAATTCGATA
ATTTGACCAAGGCTGAACGGGGTGGCCTGAGCGAGTTAGATAAGGCAGGATTTATTAAACGCCAGTTAGTGGAGACT
CGTCAAATCACCAAACATGTCGCGCAGATTTTGGACAGCCGGATGAACACCAAGTACGATGAAAATGACAAACTGAT
CCGTGAGGTGAAAGTCATTACTCTGAAGTCCAAATTAGTTAGTGATTTCCGGAAGGACTTTCAATTCTACAAAGTCC
GTGAAATTAATAACTATCATCACGCACATGACGCGTACCTGAATGCAGTGGTTGGGACCGCCCTTATCAAGAAATAT
CCTAAGCTGGAGTCGGAGTTTGTCTATGGCGACTATAAGGTATACGATGTTCGCAAAATGATTGCGAAATCTGAGCA
GGAGATCGGTAAGGCAACCGCAAAATATTTCTTTTACTCAAACATTATGAATTTCTTTAAGACAGAAATCACTCTGG
CCAACGGGGAGATTCGCAAACGTCCGTTGATCGAAACAAACGGCGAGACTGGCGAAATTGTTTGGGACAAAGGGCGT
GATTTCGCGACGGTGCGCAAGGTACTGAGCATGCCTCAAGTCAATATTGTTAAGAAAACCGAAGTGCAGACGGGCGG
GTTTTCCAAGGAAAGCATCTTACCCAAACGTAATTCAGATAAACTTATTGCACGCAAAAAGGACTGGGATCCGAAAA
AGTATGGAGGCTTCGACAGTCCAACCGTAGCCTACTCTGTTCTCGTTGTAGCGAAAGTAGAAAAGGGTAAATCCAAG
AAACTGAAATCTGTCAAGGAGTTGCTTGGAATCACCATTATGGAGCGTAGCTCCTTCGAGAAGAACCCGATTGACTT
TCTGGAAGCCAAAGGATATAAAGAGGTCAAGAAAGATCTTATCATTAAGCTGCCTAAGTATTCACTCTTCGAGCTGG
AAAATGGTCGTAAACGCATGCTCGCTTCTGCCGGCGAGTTGCAGAAGGGCAATGAATTAGCACTTCCATCAAAGTAC
GTTAACTTCCTGTATTTGGCCAGCCATTACGAGAAACTGAAGGGGTCTCCAGAGGACAACGAACAGAAACAATTATT
TGTAGAGCAGCACAAGCATTATCTTGATGAAATCATTGAGCAAATTTCCGAATTCAGTAAACGCGTAATCCTGGCCG
ATGCAAACCTCGACAAGGTGCTGAGCGCTTACAATAAGCATCGCGACAAACCTATCCGTGAGCAGGCTGAAAATATC
ATTCACCTGTTCACATTAACGAACCTGGGCGCTCCGGCCGCTTTTAAATATTTCGACACGACAATCGACCGTAAGCG
CTATACCAGTACGAAAGAAGTGTTGGATGCGACCCTTATTCACCAGTCAATTACAGGATTATATGAGACCCGTATCG
ACCTTAGCCAATTAGGTGGGGATGCGCCAGCGCCTGCTCCTGCACCCGCTCCAGCTCCTGCCCCAGCTCCAGCGCCC
GCTCCTGCGCCAGCTCCTGCACCTGCTCCAGCGATGGTGAGCAAAGGTGAAGAGGATAATATGGCCATCATCAAAGA
ATTCATGCGCTTCAAAGTTCATATGGAAGGTAGCGTTAATGGCCACGAATTTGAAATTGAAGGTGAAGGCGAAGGTC
GTCCGTATGAAGGCACCCAGACCGCAAAACTGAAAGTTACCAAAGGTGGTCCGCTGCCGTTTGCATGGGATATTCTG
AGTCCGCAGTTTATGTATGGTAGCAAAGCCTATGTTAAACATCCGGCAGATATCCCGGATTATCTGAAACTGAGCTT
TCCGGAAGGTTTTAAATGGGAACGTGTGATGAATTTTGAAGATGGTGGTGTTGTTACCGTTACACAGGATAGCAGCC
TGCAGGATGGTGAATTTATCTATAAAGTTAAACTGCGTGGCACGAATTTTCCGAGTGATGGTCCGGTTATGCAGAAA
AAAACCATGGGTTGGGAAGCAAGCAGCGAACGTATGTATCCGGAAGATGGCGCACTGAAAGGTGAAATTAAACAGCG
TCTGAAGCTGAAAGATGGCGGTCATTATGATGCAGAAGTTAAAACCACCTACAAAGCCAAAAAACCGGTTCAGCTGC
CTGGTGCATATAACGTTAACATTAAACTGGATATCACCAGCCACAACGAGGATTATACCATTGTTGAACAGTATGAA
CGTGCAGAAGGTCGCCATAGTACCGGTGGTATGGATGAACTGTATAAA
Exemplary DNA sequences encoding LbCas12a (E795L) variant protein amino
acid chimeras
(LbCas12a (E795L)-eGFP chimera DNA sequence)
SEQ ID NO: 388
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATATGGTTAGCAAA
GGTGAAGAACTGTTTACCGGCGTTGTGCCGATTCTGGTGGAACTGGATGGTGATGTGAATGGCCATAAATTTAGCGT
TAGCGGCGAAGGCGAAGGTGATGCGACCTATGGTAAACTGACCCTGAAATTTATTTGCACCACCGGTAAACTGCCGG
TTCCGTGGCCGACCCTGGTGACCACCCTGACCTATGGCGTTCAGTGCTTTAGCCGCTATCCGGATCATATGAAACAG
CATGATTTCTTTAAAAGCGCGATGCCGGAAGGCTATGTGCAGGAACGTACCATTTTTTTCAAAGATGATGGCAACTA
TAAAACCCGTGCGGAAGTTAAATTTGAAGGCGATACCCTGGTGAACCGCATTGAACTGAAAGGTATTGATTTTAAAG
AAGATGGCAACATTCTGGGTCATAAACTGGAATATAATTACAACAGCCATAATGTGTATATTATGGCCGATAAACAG
AAAAATGGCATCAAAGTGAACTTTAAAATCCGTCACAACATTGAAGATGGTAGCGTGCAGCTGGCGGATCATTATCA
GCAGAATACCCCGATTGGTGATGGCCCGGTGCTGCTGCCGGATAATCATTATCTGAGCACCCAGAGCGCGCTGAGCA
AAGATCCGAATGAAAAACGTGATCATATGGTGCTGCTGGAATTTGTTACCGCCGCGGGCATTACCCTGGGTATGGAT
GAACTGTATAAA
(LbCas12a (E795L)-GSAGSAAGSGEF(SEQ ID NO: 6)-eGFP chimera DNA sequence)
SEQ ID NO: 389
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGTAGCGCAGGT
AGTGCAGCAGGTAGCGGTGAATTTATGGTTAGCAAAGGTGAAGAACTGTTTACCGGCGTTGTGCCGATTCTGGTGGA
ACTGGATGGTGATGTGAATGGCCATAAATTTAGCGTTAGCGGCGAAGGCGAAGGTGATGCGACCTATGGTAAACTGA
CCCTGAAATTTATTTGCACCACCGGTAAACTGCCGGTTCCGTGGCCGACCCTGGTGACCACCCTGACCTATGGCGTT
CAGTGCTTTAGCCGCTATCCGGATCATATGAAACAGCATGATTTCTTTAAAAGCGCGATGCCGGAAGGCTATGTGCA
GGAACGTACCATTTTTTTCAAAGATGATGGCAACTATAAAACCCGTGCGGAAGTTAAATTTGAAGGCGATACCCTGG
TGAACCGCATTGAACTGAAAGGTATTGATTTTAAAGAAGATGGCAACATTCTGGGTCATAAACTGGAATATAATTAC
AACAGCCATAATGTGTATATTATGGCCGATAAACAGAAAAATGGCATCAAAGTGAACTTTAAAATCCGTCACAACAT
TGAAGATGGTAGCGTGCAGCTGGCGGATCATTATCAGCAGAATACCCCGATTGGTGATGGCCCGGTGCTGCTGCCGG
ATAATCATTATCTGAGCACCCAGAGCGCGCTGAGCAAAGATCCGAATGAAAAACGTGATCATATGGTGCTGCTGGAA
TTTGTTACCGCCGCGGGCATTACCCTGGGTATGGATGAACTGTATAAA
(LbCas12a (E795L)-(GGGGS)₄-(SEQ ID NO: 7)eGFP chimera DNA sequence)
SEQ ID NO: 390
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGAGGCGGAGGA
TCAGGCGGTGGCGGAAGTGGTGGAGGTGGGTCGGGAGGTGGTGGTAGCATGGTTAGCAAAGGTGAAGAACTGTTTAC
CGGCGTTGTGCCGATTCTGGTGGAACTGGATGGTGATGTGAATGGCCATAAATTTAGCGTTAGCGGCGAAGGCGAAG
GTGATGCGACCTATGGTAAACTGACCCTGAAATTTATTTGCACCACCGGTAAACTGCCGGTTCCGTGGCCGACCCTG
GTGACCACCCTGACCTATGGCGTTCAGTGCTTTAGCCGCTATCCGGATCATATGAAACAGCATGATTTCTTTAAAAG
CGCGATGCCGGAAGGCTATGTGCAGGAACGTACCATTTTTTTCAAAGATGATGGCAACTATAAAACCCGTGCGGAAG
TTAAATTTGAAGGCGATACCCTGGTGAACCGCATTGAACTGAAAGGTATTGATTTTAAAGAAGATGGCAACATTCTG
GGTCATAAACTGGAATATAATTACAACAGCCATAATGTGTATATTATGGCCGATAAACAGAAAAATGGCATCAAAGT
GAACTTTAAAATCCGTCACAACATTGAAGATGGTAGCGTGCAGCTGGCGGATCATTATCAGCAGAATACCCCGATTG
GTGATGGCCCGGTGCTGCTGCCGGATAATCATTATCTGAGCACCCAGAGCGCGCTGAGCAAAGATCCGAATGAAAAA
CGTGATCATATGGTGCTGCTGGAATTTGTTACCGCCGCGGGCATTACCCTGGGTATGGATGAACTGTATAAA
(LbCas12a (E795L)-GGGGSEAAAKGGGGS(SEQ ID NO: 37)-eGFP chimera DNA sequence)
SEQ ID NO: 391
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGAGGCGGAGGG
TCGGAAGCGGCAGCAAAAGGAGGCGGAGGAAGTATGGTTAGCAAAGGTGAAGAACTGTTTACCGGCGTTGTGCCGAT
TCTGGTGGAACTGGATGGTGATGTGAATGGCCATAAATTTAGCGTTAGCGGCGAAGGCGAAGGTGATGCGACCTATG
GTAAACTGACCCTGAAATTTATTTGCACCACCGGTAAACTGCCGGTTCCGTGGCCGACCCTGGTGACCACCCTGACC
TATGGCGTTCAGTGCTTTAGCCGCTATCCGGATCATATGAAACAGCATGATTTCTTTAAAAGCGCGATGCCGGAAGG
CTATGTGCAGGAACGTACCATTTTTTTCAAAGATGATGGCAACTATAAAACCCGTGCGGAAGTTAAATTTGAAGGCG
ATACCCTGGTGAACCGCATTGAACTGAAAGGTATTGATTTTAAAGAAGATGGCAACATTCTGGGTCATAAACTGGAA
TATAATTACAACAGCCATAATGTGTATATTATGGCCGATAAACAGAAAAATGGCATCAAAGTGAACTTTAAAATCCG
TCACAACATTGAAGATGGTAGCGTGCAGCTGGCGGATCATTATCAGCAGAATACCCCGATTGGTGATGGCCCGGTGC
TGCTGCCGGATAATCATTATCTGAGCACCCAGAGCGCGCTGAGCAAAGATCCGAATGAAAAACGTGATCATATGGTG
CTGCTGGAATTTGTTACCGCCGCGGGCATTACCCTGGGTATGGATGAACTGTATAAA
(LbCas12a (E795L)-A(EAAAK)₄A-(SEQ ID NO: 8)eGFP chimera DNA sequence)
SEQ ID NO: 392
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTGAGGCCGCA
GCAAAAGAGGCTGCCGCTAAGGAGGCTGCTGCAAAAGAAGCAGCAGCGAAAGCTATGGTTAGCAAAGGTGAAGAACT
GTTTACCGGCGTTGTGCCGATTCTGGTGGAACTGGATGGTGATGTGAATGGCCATAAATTTAGCGTTAGCGGCGAAG
GCGAAGGTGATGCGACCTATGGTAAACTGACCCTGAAATTTATTTGCACCACCGGTAAACTGCCGGTTCCGTGGCCG
ACCCTGGTGACCACCCTGACCTATGGCGTTCAGTGCTTTAGCCGCTATCCGGATCATATGAAACAGCATGATTTCTT
TAAAAGCGCGATGCCGGAAGGCTATGTGCAGGAACGTACCATTTTTTTCAAAGATGATGGCAACTATAAAACCCGTG
CGGAAGTTAAATTTGAAGGCGATACCCTGGTGAACCGCATTGAACTGAAAGGTATTGATTTTAAAGAAGATGGCAAC
ATTCTGGGTCATAAACTGGAATATAATTACAACAGCCATAATGTGTATATTATGGCCGATAAACAGAAAAATGGCAT
CAAAGTGAACTTTAAAATCCGTCACAACATTGAAGATGGTAGCGTGCAGCTGGCGGATCATTATCAGCAGAATACCC
CGATTGGTGATGGCCCGGTGCTGCTGCCGGATAATCATTATCTGAGCACCCAGAGCGCGCTGAGCAAAGATCCGAAT
GAAAAACGTGATCATATGGTGCTGCTGGAATTTGTTACCGCCGCGGGCATTACCCTGGGTATGGATGAACTGTATAA
A
(LbCas12a (E795L)-A(EAAAK)₄ALEA(EAAAK)₄A(SEQ ID NO: 9)-
eGFP chimera DNA sequence)
SEQ ID NO: 393
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCCGAAGCAGCA
GCCAAAGAAGCTGCCGCAAAGGAGGCGGCAGCTAAAGAAGCGGCCGCTAAGGCATTGGAAGCGGAGGCGGCTGCCAA
GGAGGCTGCGGCGAAAGAAGCTGCGGCCAAAGAAGCGGCTGCAAAAGCGATGGTTAGCAAAGGTGAAGAACTGTTTA
CCGGCGTTGTGCCGATTCTGGTGGAACTGGATGGTGATGTGAATGGCCATAAATTTAGCGTTAGCGGCGAAGGCGAA
GGTGATGCGACCTATGGTAAACTGACCCTGAAATTTATTTGCACCACCGGTAAACTGCCGGTTCCGTGGCCGACCCT
GGTGACCACCCTGACCTATGGCGTTCAGTGCTTTAGCCGCTATCCGGATCATATGAAACAGCATGATTTCTTTAAAA
GCGCGATGCCGGAAGGCTATGTGCAGGAACGTACCATTTTTTTCAAAGATGATGGCAACTATAAAACCCGTGCGGAA
GTTAAATTTGAAGGCGATACCCTGGTGAACCGCATTGAACTGAAAGGTATTGATTTTAAAGAAGATGGCAACATTCT
GGGTCATAAACTGGAATATAATTACAACAGCCATAATGTGTATATTATGGCCGATAAACAGAAAAATGGCATCAAAG
TGAACTTTAAAATCCGTCACAACATTGAAGATGGTAGCGTGCAGCTGGCGGATCATTATCAGCAGAATACCCCGATT
GGTGATGGCCCGGTGCTGCTGCCGGATAATCATTATCTGAGCACCCAGAGCGCGCTGAGCAAAGATCCGAATGAAAA
ACGTGATCATATGGTGCTGCTGGAATTTGTTACCGCCGCGGGCATTACCCTGGGTATGGATGAACTGTATAAA
(LbCas12a (E795L)-LEA(EAAAK)₄ALEA(EAAAK)₄ALE(SEQ ID NO: 10)-
eGFP chimera DNA sequence)
SEQ ID NO: 394
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATTTAGAGGCTGAG
GCTGCTGCCAAGGAGGCTGCTGCGAAGGAAGCTGCAGCTAAAGAGGCAGCGGCCAAAGCACTTGAGGCCGAAGCCGC
CGCGAAAGAAGCCGCTGCTAAGGAGGCAGCCGCCAAAGAAGCGGCAGCTAAAGCATTAGAAATGGTTAGCAAAGGTG
AAGAACTGTTTACCGGCGTTGTGCCGATTCTGGTGGAACTGGATGGTGATGTGAATGGCCATAAATTTAGCGTTAGC
GGCGAAGGCGAAGGTGATGCGACCTATGGTAAACTGACCCTGAAATTTATTTGCACCACCGGTAAACTGCCGGTTCC
GTGGCCGACCCTGGTGACCACCCTGACCTATGGCGTTCAGTGCTTTAGCCGCTATCCGGATCATATGAAACAGCATG
ATTTCTTTAAAAGCGCGATGCCGGAAGGCTATGTGCAGGAACGTACCATTTTTTTCAAAGATGATGGCAACTATAAA
ACCCGTGCGGAAGTTAAATTTGAAGGCGATACCCTGGTGAACCGCATTGAACTGAAAGGTATTGATTTTAAAGAAGA
TGGCAACATTCTGGGTCATAAACTGGAATATAATTACAACAGCCATAATGTGTATATTATGGCCGATAAACAGAAAA
ATGGCATCAAAGTGAACTTTAAAATCCGTCACAACATTGAAGATGGTAGCGTGCAGCTGGCGGATCATTATCAGCAG
AATACCCCGATTGGTGATGGCCCGGTGCTGCTGCCGGATAATCATTATCTGAGCACCCAGAGCGCGCTGAGCAAAGA
TCCGAATGAAAAACGTGATCATATGGTGCTGCTGGAATTTGTTACCGCCGCGGGCATTACCCTGGGTATGGATGAAC
TGTATAAA
(LbCas12a (E795L)-APA-eGFP chimera DNA sequence)
SEQ ID NO: 395
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCACCTGCGATG
GTTAGCAAAGGTGAAGAACTGTTTACCGGCGTTGTGCCGATTCTGGTGGAACTGGATGGTGATGTGAATGGCCATAA
ATTTAGCGTTAGCGGCGAAGGCGAAGGTGATGCGACCTATGGTAAACTGACCCTGAAATTTATTTGCACCACCGGTA
AACTGCCGGTTCCGTGGCCGACCCTGGTGACCACCCTGACCTATGGCGTTCAGTGCTTTAGCCGCTATCCGGATCAT
ATGAAACAGCATGATTTCTTTAAAAGCGCGATGCCGGAAGGCTATGTGCAGGAACGTACCATTTTTTTCAAAGATGA
TGGCAACTATAAAACCCGTGCGGAAGTTAAATTTGAAGGCGATACCCTGGTGAACCGCATTGAACTGAAAGGTATTG
ATTTTAAAGAAGATGGCAACATTCTGGGTCATAAACTGGAATATAATTACAACAGCCATAATGTGTATATTATGGCC
GATAAACAGAAAAATGGCATCAAAGTGAACTTTAAAATCCGTCACAACATTGAAGATGGTAGCGTGCAGCTGGCGGA
TCATTATCAGCAGAATACCCCGATTGGTGATGGCCCGGTGCTGCTGCCGGATAATCATTATCTGAGCACCCAGAGCG
CGCTGAGCAAAGATCCGAATGAAAAACGTGATCATATGGTGCTGCTGGAATTTGTTACCGCCGCGGGCATTACCCTG
GGTATGGATGAACTGTATAAA
(LbCas12a (E795L)-APAPA(SEQ ID NO: 11)-eGFP chimera DNA sequence)
SEQ ID NO: 396
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTCCTGCACCT
GCGATGGTTAGCAAAGGTGAAGAACTGTTTACCGGCGTTGTGCCGATTCTGGTGGAACTGGATGGTGATGTGAATGG
CCATAAATTTAGCGTTAGCGGCGAAGGCGAAGGTGATGCGACCTATGGTAAACTGACCCTGAAATTTATTTGCACCA
CCGGTAAACTGCCGGTTCCGTGGCCGACCCTGGTGACCACCCTGACCTATGGCGTTCAGTGCTTTAGCCGCTATCCG
GATCATATGAAACAGCATGATTTCTTTAAAAGCGCGATGCCGGAAGGCTATGTGCAGGAACGTACCATTTTTTTCAA
AGATGATGGCAACTATAAAACCCGTGCGGAAGTTAAATTTGAAGGCGATACCCTGGTGAACCGCATTGAACTGAAAG
GTATTGATTTTAAAGAAGATGGCAACATTCTGGGTCATAAACTGGAATATAATTACAACAGCCATAATGTGTATATT
ATGGCCGATAAACAGAAAAATGGCATCAAAGTGAACTTTAAAATCCGTCACAACATTGAAGATGGTAGCGTGCAGCT
GGCGGATCATTATCAGCAGAATACCCCGATTGGTGATGGCCCGGTGCTGCTGCCGGATAATCATTATCTGAGCACCC
AGAGCGCGCTGAGCAAAGATCCGAATGAAAAACGTGATCATATGGTGCTGCTGGAATTTGTTACCGCCGCGGGCATT
ACCCTGGGTATGGATGAACTGTATAAA
(LbCas12a (E795L)-APAPAPA(SEQ ID NO: 12)-eGFP chimera DNA sequence)
SEQ ID NO: 397
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCTCCT
GCACCTGCGATGGTTAGCAAAGGTGAAGAACTGTTTACCGGCGTTGTGCCGATTCTGGTGGAACTGGATGGTGATGT
GAATGGCCATAAATTTAGCGTTAGCGGCGAAGGCGAAGGTGATGCGACCTATGGTAAACTGACCCTGAAATTTATTT
GCACCACCGGTAAACTGCCGGTTCCGTGGCCGACCCTGGTGACCACCCTGACCTATGGCGTTCAGTGCTTTAGCCGC
TATCCGGATCATATGAAACAGCATGATTTCTTTAAAAGCGCGATGCCGGAAGGCTATGTGCAGGAACGTACCATTTT
TTTCAAAGATGATGGCAACTATAAAACCCGTGCGGAAGTTAAATTTGAAGGCGATACCCTGGTGAACCGCATTGAAC
TGAAAGGTATTGATTTTAAAGAAGATGGCAACATTCTGGGTCATAAACTGGAATATAATTACAACAGCCATAATGTG
TATATTATGGCCGATAAACAGAAAAATGGCATCAAAGTGAACTTTAAAATCCGTCACAACATTGAAGATGGTAGCGT
GCAGCTGGCGGATCATTATCAGCAGAATACCCCGATTGGTGATGGCCCGGTGCTGCTGCCGGATAATCATTATCTGA
GCACCCAGAGCGCGCTGAGCAAAGATCCGAATGAAAAACGTGATCATATGGTGCTGCTGGAATTTGTTACCGCCGCG
GGCATTACCCTGGGTATGGATGAACTGTATAAA
(LbCas12a(E795L)-APAPAPAPA(SEQ ID NO: 13)-eGFP chimera DNA sequence)
SEQ ID NO: 398
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTCCTGCGCCA
GCTCCTGCACCTGCGATGGTTAGCAAAGGTGAAGAACTGTTTACCGGCGTTGTGCCGATTCTGGTGGAACTGGATGG
TGATGTGAATGGCCATAAATTTAGCGTTAGCGGCGAAGGCGAAGGTGATGCGACCTATGGTAAACTGACCCTGAAAT
TTATTTGCACCACCGGTAAACTGCCGGTTCCGTGGCCGACCCTGGTGACCACCCTGACCTATGGCGTTCAGTGCTTT
AGCCGCTATCCGGATCATATGAAACAGCATGATTTCTTTAAAAGCGCGATGCCGGAAGGCTATGTGCAGGAACGTAC
CATTTTTTTCAAAGATGATGGCAACTATAAAACCCGTGCGGAAGTTAAATTTGAAGGCGATACCCTGGTGAACCGCA
TTGAACTGAAAGGTATTGATTTTAAAGAAGATGGCAACATTCTGGGTCATAAACTGGAATATAATTACAACAGCCAT
AATGTGTATATTATGGCCGATAAACAGAAAAATGGCATCAAAGTGAACTTTAAAATCCGTCACAACATTGAAGATGG
TAGCGTGCAGCTGGCGGATCATTATCAGCAGAATACCCCGATTGGTGATGGCCCGGTGCTGCTGCCGGATAATCATT
ATCTGAGCACCCAGAGCGCGCTGAGCAAAGATCCGAATGAAAAACGTGATCATATGGTGCTGCTGGAATTTGTTACC
GCCGCGGGCATTACCCTGGGTATGGATGAACTGTATAAA
(LbCas12a (E795L)-APAPAPAPAPA(SEQ ID NO: 14)-eGFP chimera DNA sequence)
SEQ ID NO: 399
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCCGCTCCT
GCGCCAGCTCCTGCACCTGCGATGGTTAGCAAAGGTGAAGAACTGTTTACCGGCGTTGTGCCGATTCTGGTGGAACT
GGATGGTGATGTGAATGGCCATAAATTTAGCGTTAGCGGCGAAGGCGAAGGTGATGCGACCTATGGTAAACTGACCC
TGAAATTTATTTGCACCACCGGTAAACTGCCGGTTCCGTGGCCGACCCTGGTGACCACCCTGACCTATGGCGTTCAG
TGCTTTAGCCGCTATCCGGATCATATGAAACAGCATGATTTCTTTAAAAGCGCGATGCCGGAAGGCTATGTGCAGGA
ACGTACCATTTTTTTCAAAGATGATGGCAACTATAAAACCCGTGCGGAAGTTAAATTTGAAGGCGATACCCTGGTGA
ACCGCATTGAACTGAAAGGTATTGATTTTAAAGAAGATGGCAACATTCTGGGTCATAAACTGGAATATAATTACAAC
AGCCATAATGTGTATATTATGGCCGATAAACAGAAAAATGGCATCAAAGTGAACTTTAAAATCCGTCACAACATTGA
AGATGGTAGCGTGCAGCTGGCGGATCATTATCAGCAGAATACCCCGATTGGTGATGGCCCGGTGCTGCTGCCGGATA
ATCATTATCTGAGCACCCAGAGCGCGCTGAGCAAAGATCCGAATGAAAAACGTGATCATATGGTGCTGCTGGAATTT
GTTACCGCCGCGGGCATTACCCTGGGTATGGATGAACTGTATAAA
(LbCas12a (E795L)-APAPAPAPAPAPA(SEQ ID NO: 15)-eGFP chimera DNA sequence)
SEQ ID NO: 400
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCACCTGCGATGGTTAGCAAAGGTGAAGAACTGTTTACCGGCGTTGTGCCGATTCTGGT
GGAACTGGATGGTGATGTGAATGGCCATAAATTTAGCGTTAGCGGCGAAGGCGAAGGTGATGCGACCTATGGTAAAC
TGACCCTGAAATTTATTTGCACCACCGGTAAACTGCCGGTTCCGTGGCCGACCCTGGTGACCACCCTGACCTATGGC
GTTCAGTGCTTTAGCCGCTATCCGGATCATATGAAACAGCATGATTTCTTTAAAAGCGCGATGCCGGAAGGCTATGT
GCAGGAACGTACCATTTTTTTCAAAGATGATGGCAACTATAAAACCCGTGCGGAAGTTAAATTTGAAGGCGATACCC
TGGTGAACCGCATTGAACTGAAAGGTATTGATTTTAAAGAAGATGGCAACATTCTGGGTCATAAACTGGAATATAAT
TACAACAGCCATAATGTGTATATTATGGCCGATAAACAGAAAAATGGCATCAAAGTGAACTTTAAAATCCGTCACAA
CATTGAAGATGGTAGCGTGCAGCTGGCGGATCATTATCAGCAGAATACCCCGATTGGTGATGGCCCGGTGCTGCTGC
CGGATAATCATTATCTGAGCACCCAGAGCGCGCTGAGCAAAGATCCGAATGAAAAACGTGATCATATGGTGCTGCTG
GAATTTGTTACCGCCGCGGGCATTACCCTGGGTATGGATGAACTGTATAAA
(LbCas12a (E795L)-APAPAPAPAPAPAPA(SEQ ID NO: 16)-eGFP chimera DNA sequence)
SEQ ID NO: 401
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCGATGGTTAGCAAAGGTGAAGAACTGTTTACCGGCGTTGTGCCGAT
TCTGGTGGAACTGGATGGTGATGTGAATGGCCATAAATTTAGCGTTAGCGGCGAAGGCGAAGGTGATGCGACCTATG
GTAAACTGACCCTGAAATTTATTTGCACCACCGGTAAACTGCCGGTTCCGTGGCCGACCCTGGTGACCACCCTGACC
TATGGCGTTCAGTGCTTTAGCCGCTATCCGGATCATATGAAACAGCATGATTTCTTTAAAAGCGCGATGCCGGAAGG
CTATGTGCAGGAACGTACCATTTTTTTCAAAGATGATGGCAACTATAAAACCCGTGCGGAAGTTAAATTTGAAGGCG
ATACCCTGGTGAACCGCATTGAACTGAAAGGTATTGATTTTAAAGAAGATGGCAACATTCTGGGTCATAAACTGGAA
TATAATTACAACAGCCATAATGTGTATATTATGGCCGATAAACAGAAAAATGGCATCAAAGTGAACTTTAAAATCCG
TCACAACATTGAAGATGGTAGCGTGCAGCTGGCGGATCATTATCAGCAGAATACCCCGATTGGTGATGGCCCGGTGC
TGCTGCCGGATAATCATTATCTGAGCACCCAGAGCGCGCTGAGCAAAGATCCGAATGAAAAACGTGATCATATGGTG
CTGCTGGAATTTGTTACCGCCGCGGGCATTACCCTGGGTATGGATGAACTGTATAAA
(LbCas12a (E795L)-APAPAPAPAPAPAPAPA(SEQ ID NO: 17)-eGFP chimera DNA sequence)
SEQ ID NO: 402
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCGCCAGCTCCTGCACCTGCGATGGTTAGCAAAGGTGAAGAACTGTTTACCGGCGTTGT
GCCGATTCTGGTGGAACTGGATGGTGATGTGAATGGCCATAAATTTAGCGTTAGCGGCGAAGGCGAAGGTGATGCGA
CCTATGGTAAACTGACCCTGAAATTTATTTGCACCACCGGTAAACTGCCGGTTCCGTGGCCGACCCTGGTGACCACC
CTGACCTATGGCGTTCAGTGCTTTAGCCGCTATCCGGATCATATGAAACAGCATGATTTCTTTAAAAGCGCGATGCC
GGAAGGCTATGTGCAGGAACGTACCATTTTTTTCAAAGATGATGGCAACTATAAAACCCGTGCGGAAGTTAAATTTG
AAGGCGATACCCTGGTGAACCGCATTGAACTGAAAGGTATTGATTTTAAAGAAGATGGCAACATTCTGGGTCATAAA
CTGGAATATAATTACAACAGCCATAATGTGTATATTATGGCCGATAAACAGAAAAATGGCATCAAAGTGAACTTTAA
AATCCGTCACAACATTGAAGATGGTAGCGTGCAGCTGGCGGATCATTATCAGCAGAATACCCCGATTGGTGATGGCC
CGGTGCTGCTGCCGGATAATCATTATCTGAGCACCCAGAGCGCGCTGAGCAAAGATCCGAATGAAAAACGTGATCAT
ATGGTGCTGCTGGAATTTGTTACCGCCGCGGGCATTACCCTGGGTATGGATGAACTGTATAAA
(LbCas12a (E795L)-APAPAPAPAPAPAPAPAPA(SEQ ID NO: 18)-
eGFP chimera DNA sequence)
SEQ ID NO: 403
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCGCCAGCTCCTGCACCTGCGATGGTTAGCAAAGGTGAAGAACTGTTTACCGG
CGTTGTGCCGATTCTGGTGGAACTGGATGGTGATGTGAATGGCCATAAATTTAGCGTTAGCGGCGAAGGCGAAGGTG
ATGCGACCTATGGTAAACTGACCCTGAAATTTATTTGCACCACCGGTAAACTGCCGGTTCCGTGGCCGACCCTGGTG
ACCACCCTGACCTATGGCGTTCAGTGCTTTAGCCGCTATCCGGATCATATGAAACAGCATGATTTCTTTAAAAGCGC
GATGCCGGAAGGCTATGTGCAGGAACGTACCATTTTTTTCAAAGATGATGGCAACTATAAAACCCGTGCGGAAGTTA
AATTTGAAGGCGATACCCTGGTGAACCGCATTGAACTGAAAGGTATTGATTTTAAAGAAGATGGCAACATTCTGGGT
CATAAACTGGAATATAATTACAACAGCCATAATGTGTATATTATGGCCGATAAACAGAAAAATGGCATCAAAGTGAA
CTTTAAAATCCGTCACAACATTGAAGATGGTAGCGTGCAGCTGGCGGATCATTATCAGCAGAATACCCCGATTGGTG
ATGGCCCGGTGCTGCTGCCGGATAATCATTATCTGAGCACCCAGAGCGCGCTGAGCAAAGATCCGAATGAAAAACGT
GATCATATGGTGCTGCTGGAATTTGTTACCGCCGCGGGCATTACCCTGGGTATGGATGAACTGTATAAA
(LbCas12a (E795L)-APAPAPAPAPAPAPAPAPAPA(SEQ ID NO: 19)-
eGFP chimera DNA sequence)
SEQ ID NO: 404
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCTCCTGCGCCAGCTCCTGCACCTGCGATGGTTAGCAAAGGTGAAGAACTGTT
TACCGGCGTTGTGCCGATTCTGGTGGAACTGGATGGTGATGTGAATGGCCATAAATTTAGCGTTAGCGGCGAAGGCG
AAGGTGATGCGACCTATGGTAAACTGACCCTGAAATTTATTTGCACCACCGGTAAACTGCCGGTTCCGTGGCCGACC
CTGGTGACCACCCTGACCTATGGCGTTCAGTGCTTTAGCCGCTATCCGGATCATATGAAACAGCATGATTTCTTTAA
AAGCGCGATGCCGGAAGGCTATGTGCAGGAACGTACCATTTTTTTCAAAGATGATGGCAACTATAAAACCCGTGCGG
AAGTTAAATTTGAAGGCGATACCCTGGTGAACCGCATTGAACTGAAAGGTATTGATTTTAAAGAAGATGGCAACATT
CTGGGTCATAAACTGGAATATAATTACAACAGCCATAATGTGTATATTATGGCCGATAAACAGAAAAATGGCATCAA
AGTGAACTTTAAAATCCGTCACAACATTGAAGATGGTAGCGTGCAGCTGGCGGATCATTATCAGCAGAATACCCCGA
TTGGTGATGGCCCGGTGCTGCTGCCGGATAATCATTATCTGAGCACCCAGAGCGCGCTGAGCAAAGATCCGAATGAA
AAACGTGATCATATGGTGCTGCTGGAATTTGTTACCGCCGCGGGCATTACCCTGGGTATGGATGAACTGTATAAA
(LbCas12a (E795L)-APAPAPAPAPAPAPAPAPAPAPA(SEQ ID NO: 20)-
eGFP chimera DNA sequence)
SEQ ID NO: 405
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCACCTGCGATGGTTAGCAAAGGTGAAGA
ACTGTTTACCGGCGTTGTGCCGATTCTGGTGGAACTGGATGGTGATGTGAATGGCCATAAATTTAGCGTTAGCGGCG
AAGGCGAAGGTGATGCGACCTATGGTAAACTGACCCTGAAATTTATTTGCACCACCGGTAAACTGCCGGTTCCGTGG
CCGACCCTGGTGACCACCCTGACCTATGGCGTTCAGTGCTTTAGCCGCTATCCGGATCATATGAAACAGCATGATTT
CTTTAAAAGCGCGATGCCGGAAGGCTATGTGCAGGAACGTACCATTTTTTTCAAAGATGATGGCAACTATAAAACCC
GTGCGGAAGTTAAATTTGAAGGCGATACCCTGGTGAACCGCATTGAACTGAAAGGTATTGATTTTAAAGAAGATGGC
AACATTCTGGGTCATAAACTGGAATATAATTACAACAGCCATAATGTGTATATTATGGCCGATAAACAGAAAAATGG
CATCAAAGTGAACTTTAAAATCCGTCACAACATTGAAGATGGTAGCGTGCAGCTGGCGGATCATTATCAGCAGAATA
CCCCGATTGGTGATGGCCCGGTGCTGCTGCCGGATAATCATTATCTGAGCACCCAGAGCGCGCTGAGCAAAGATCCG
AATGAAAAACGTGATCATATGGTGCTGCTGGAATTTGTTACCGCCGCGGGCATTACCCTGGGTATGGATGAACTGTA
TAAA
(LbCas12a (E795L)-APAPAPAPAPAPAPAPAPAPAPAPA(SEQ ID NO: 21)-
eGFP chimera DNA sequence)
SEQ ID NO: 406
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCTCCTGCACCTGCGATGGTTAGCAAAGG
TGAAGAACTGTTTACCGGCGTTGTGCCGATTCTGGTGGAACTGGATGGTGATGTGAATGGCCATAAATTTAGCGTTA
GCGGCGAAGGCGAAGGTGATGCGACCTATGGTAAACTGACCCTGAAATTTATTTGCACCACCGGTAAACTGCCGGTT
CCGTGGCCGACCCTGGTGACCACCCTGACCTATGGCGTTCAGTGCTTTAGCCGCTATCCGGATCATATGAAACAGCA
TGATTTCTTTAAAAGCGCGATGCCGGAAGGCTATGTGCAGGAACGTACCATTTTTTTCAAAGATGATGGCAACTATA
AAACCCGTGCGGAAGTTAAATTTGAAGGCGATACCCTGGTGAACCGCATTGAACTGAAAGGTATTGATTTTAAAGAA
GATGGCAACATTCTGGGTCATAAACTGGAATATAATTACAACAGCCATAATGTGTATATTATGGCCGATAAACAGAA
AAATGGCATCAAAGTGAACTTTAAAATCCGTCACAACATTGAAGATGGTAGCGTGCAGCTGGCGGATCATTATCAGC
AGAATACCCCGATTGGTGATGGCCCGGTGCTGCTGCCGGATAATCATTATCTGAGCACCCAGAGCGCGCTGAGCAAA
GATCCGAATGAAAAACGTGATCATATGGTGCTGCTGGAATTTGTTACCGCCGCGGGCATTACCCTGGGTATGGATGA
ACTGTATAAA
(LbCas12a (E795L)-APAPAPAPAPAPAPAPAPAPAPAPAPA(SEQ ID NO: 22)-
eGFP chimera DNA sequence)
SEQ ID NO: 407
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCGCCAGCTCCTGCACCTGCGATGGTTAG
CAAAGGTGAAGAACTGTTTACCGGCGTTGTGCCGATTCTGGTGGAACTGGATGGTGATGTGAATGGCCATAAATTTA
GCGTTAGCGGCGAAGGCGAAGGTGATGCGACCTATGGTAAACTGACCCTGAAATTTATTTGCACCACCGGTAAACTG
CCGGTTCCGTGGCCGACCCTGGTGACCACCCTGACCTATGGCGTTCAGTGCTTTAGCCGCTATCCGGATCATATGAA
ACAGCATGATTTCTTTAAAAGCGCGATGCCGGAAGGCTATGTGCAGGAACGTACCATTTTTTTCAAAGATGATGGCA
ACTATAAAACCCGTGCGGAAGTTAAATTTGAAGGCGATACCCTGGTGAACCGCATTGAACTGAAAGGTATTGATTTT
AAAGAAGATGGCAACATTCTGGGTCATAAACTGGAATATAATTACAACAGCCATAATGTGTATATTATGGCCGATAA
ACAGAAAAATGGCATCAAAGTGAACTTTAAAATCCGTCACAACATTGAAGATGGTAGCGTGCAGCTGGCGGATCATT
ATCAGCAGAATACCCCGATTGGTGATGGCCCGGTGCTGCTGCCGGATAATCATTATCTGAGCACCCAGAGCGCGCTG
AGCAAAGATCCGAATGAAAAACGTGATCATATGGTGCTGCTGGAATTTGTTACCGCCGCGGGCATTACCCTGGGTAT
GGATGAACTGTATAAA
(LbCas12a (E795L)-APAPAPAPAPAPAPAPAPAPAPAPAPAPA(SEQ ID NO: 23)-
eGFP chimera DNA sequence)
SEQ ID NO: 408
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCGCCAGCTCCTGCACCTGCTCCAGCGAT
GGTTAGCAAAGGTGAAGAACTGTTTACCGGCGTTGTGCCGATTCTGGTGGAACTGGATGGTGATGTGAATGGCCATA
AATTTAGCGTTAGCGGCGAAGGCGAAGGTGATGCGACCTATGGTAAACTGACCCTGAAATTTATTTGCACCACCGGT
AAACTGCCGGTTCCGTGGCCGACCCTGGTGACCACCCTGACCTATGGCGTTCAGTGCTTTAGCCGCTATCCGGATCA
TATGAAACAGCATGATTTCTTTAAAAGCGCGATGCCGGAAGGCTATGTGCAGGAACGTACCATTTTTTTCAAAGATG
ATGGCAACTATAAAACCCGTGCGGAAGTTAAATTTGAAGGCGATACCCTGGTGAACCGCATTGAACTGAAAGGTATT
GATTTTAAAGAAGATGGCAACATTCTGGGTCATAAACTGGAATATAATTACAACAGCCATAATGTGTATATTATGGC
CGATAAACAGAAAAATGGCATCAAAGTGAACTTTAAAATCCGTCACAACATTGAAGATGGTAGCGTGCAGCTGGCGG
ATCATTATCAGCAGAATACCCCGATTGGTGATGGCCCGGTGCTGCTGCCGGATAATCATTATCTGAGCACCCAGAGC
GCGCTGAGCAAAGATCCGAATGAAAAACGTGATCATATGGTGCTGCTGGAATTTGTTACCGCCGCGGGCATTACCCT
GGGTATGGATGAACTGTATAAA
(LbCas12a (E795L)-mCherry chimera DNA sequence)
SEQ ID NO: 409
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATATGGTGAGCAAA
GGTGAAGAGGATAATATGGCCATCATCAAAGAATTCATGCGCTTCAAAGTTCATATGGAAGGTAGCGTTAATGGCCA
CGAATTTGAAATTGAAGGTGAAGGCGAAGGTCGTCCGTATGAAGGCACCCAGACCGCAAAACTGAAAGTTACCAAAG
GTGGTCCGCTGCCGTTTGCATGGGATATTCTGAGTCCGCAGTTTATGTATGGTAGCAAAGCCTATGTTAAACATCCG
GCAGATATCCCGGATTATCTGAAACTGAGCTTTCCGGAAGGTTTTAAATGGGAACGTGTGATGAATTTTGAAGATGG
TGGTGTTGTTACCGTTACACAGGATAGCAGCCTGCAGGATGGTGAATTTATCTATAAAGTTAAACTGCGTGGCACGA
ATTTTCCGAGTGATGGTCCGGTTATGCAGAAAAAAACCATGGGTTGGGAAGCAAGCAGCGAACGTATGTATCCGGAA
GATGGCGCACTGAAAGGTGAAATTAAACAGCGTCTGAAGCTGAAAGATGGCGGTCATTATGATGCAGAAGTTAAAAC
CACCTACAAAGCCAAAAAACCGGTTCAGCTGCCTGGTGCATATAACGTTAACATTAAACTGGATATCACCAGCCACA
ACGAGGATTATACCATTGTTGAACAGTATGAACGTGCAGAAGGTCGCCATAGTACCGGTGGTATGGATGAACTGTAT
AAA
(LbCas12a (E795L)-GSAGSAAGSGEF(SEQ ID NO: 6)-mCherry chimera DNA sequence)
SEQ ID NO: 410
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGTAGCGCAGGT
AGTGCAGCAGGTAGCGGTGAATTTATGGTGAGCAAAGGTGAAGAGGATAATATGGCCATCATCAAAGAATTCATGCG
CTTCAAAGTTCATATGGAAGGTAGCGTTAATGGCCACGAATTTGAAATTGAAGGTGAAGGCGAAGGTCGTCCGTATG
AAGGCACCCAGACCGCAAAACTGAAAGTTACCAAAGGTGGTCCGCTGCCGTTTGCATGGGATATTCTGAGTCCGCAG
TTTATGTATGGTAGCAAAGCCTATGTTAAACATCCGGCAGATATCCCGGATTATCTGAAACTGAGCTTTCCGGAAGG
TTTTAAATGGGAACGTGTGATGAATTTTGAAGATGGTGGTGTTGTTACCGTTACACAGGATAGCAGCCTGCAGGATG
GTGAATTTATCTATAAAGTTAAACTGCGTGGCACGAATTTTCCGAGTGATGGTCCGGTTATGCAGAAAAAAACCATG
GGTTGGGAAGCAAGCAGCGAACGTATGTATCCGGAAGATGGCGCACTGAAAGGTGAAATTAAACAGCGTCTGAAGCT
GAAAGATGGCGGTCATTATGATGCAGAAGTTAAAACCACCTACAAAGCCAAAAAACCGGTTCAGCTGCCTGGTGCAT
ATAACGTTAACATTAAACTGGATATCACCAGCCACAACGAGGATTATACCATTGTTGAACAGTATGAACGTGCAGAA
GGTCGCCATAGTACCGGTGGTATGGATGAACTGTATAAA
(LbCas12a (E795L)-(GGGGS)₄-(SEQ ID NO: 7)mCherry chimera DNA sequence)
SEQ ID NO: 411
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGAGGCGGAGGA
TCAGGCGGTGGCGGAAGTGGTGGAGGTGGGTCGGGAGGTGGTGGTAGCATGGTGAGCAAAGGTGAAGAGGATAATAT
GGCCATCATCAAAGAATTCATGCGCTTCAAAGTTCATATGGAAGGTAGCGTTAATGGCCACGAATTTGAAATTGAAG
GTGAAGGCGAAGGTCGTCCGTATGAAGGCACCCAGACCGCAAAACTGAAAGTTACCAAAGGTGGTCCGCTGCCGTTT
GCATGGGATATTCTGAGTCCGCAGTTTATGTATGGTAGCAAAGCCTATGTTAAACATCCGGCAGATATCCCGGATTA
TCTGAAACTGAGCTTTCCGGAAGGTTTTAAATGGGAACGTGTGATGAATTTTGAAGATGGTGGTGTTGTTACCGTTA
CACAGGATAGCAGCCTGCAGGATGGTGAATTTATCTATAAAGTTAAACTGCGTGGCACGAATTTTCCGAGTGATGGT
CCGGTTATGCAGAAAAAAACCATGGGTTGGGAAGCAAGCAGCGAACGTATGTATCCGGAAGATGGCGCACTGAAAGG
TGAAATTAAACAGCGTCTGAAGCTGAAAGATGGCGGTCATTATGATGCAGAAGTTAAAACCACCTACAAAGCCAAAA
AACCGGTTCAGCTGCCTGGTGCATATAACGTTAACATTAAACTGGATATCACCAGCCACAACGAGGATTATACCATT
GTTGAACAGTATGAACGTGCAGAAGGTCGCCATAGTACCGGTGGTATGGATGAACTGTATAAA
(LbCas12a (E795L)-GGGGSEAAAKGGGGS(SEQ ID NO: 37)-
mCherry chimera DNA sequence)
SEQ ID NO: 412
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGGAGGCGGAGGG
TCGGAAGCGGCAGCAAAAGGAGGCGGAGGAAGTATGGTGAGCAAAGGTGAAGAGGATAATATGGCCATCATCAAAGA
ATTCATGCGCTTCAAAGTTCATATGGAAGGTAGCGTTAATGGCCACGAATTTGAAATTGAAGGTGAAGGCGAAGGTC
GTCCGTATGAAGGCACCCAGACCGCAAAACTGAAAGTTACCAAAGGTGGTCCGCTGCCGTTTGCATGGGATATTCTG
AGTCCGCAGTTTATGTATGGTAGCAAAGCCTATGTTAAACATCCGGCAGATATCCCGGATTATCTGAAACTGAGCTT
TCCGGAAGGTTTTAAATGGGAACGTGTGATGAATTTTGAAGATGGTGGTGTTGTTACCGTTACACAGGATAGCAGCC
TGCAGGATGGTGAATTTATCTATAAAGTTAAACTGCGTGGCACGAATTTTCCGAGTGATGGTCCGGTTATGCAGAAA
AAAACCATGGGTTGGGAAGCAAGCAGCGAACGTATGTATCCGGAAGATGGCGCACTGAAAGGTGAAATTAAACAGCG
TCTGAAGCTGAAAGATGGCGGTCATTATGATGCAGAAGTTAAAACCACCTACAAAGCCAAAAAACCGGTTCAGCTGC
CTGGTGCATATAACGTTAACATTAAACTGGATATCACCAGCCACAACGAGGATTATACCATTGTTGAACAGTATGAA
CGTGCAGAAGGTCGCCATAGTACCGGTGGTATGGATGAACTGTATAAA
(LbCas12a (E795L)-A(EAAAK)₄A-(SEQ ID NO: 8)mCherry chimera DNA sequence)
SEQ ID NO: 413
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTGAGGCCGCA
GCAAAAGAGGCTGCCGCTAAGGAGGCTGCTGCAAAAGAAGCAGCAGCGAAAGCTATGGTGAGCAAAGGTGAAGAGGA
TAATATGGCCATCATCAAAGAATTCATGCGCTTCAAAGTTCATATGGAAGGTAGCGTTAATGGCCACGAATTTGAAA
TTGAAGGTGAAGGCGAAGGTCGTCCGTATGAAGGCACCCAGACCGCAAAACTGAAAGTTACCAAAGGTGGTCCGCTG
CCGTTTGCATGGGATATTCTGAGTCCGCAGTTTATGTATGGTAGCAAAGCCTATGTTAAACATCCGGCAGATATCCC
GGATTATCTGAAACTGAGCTTTCCGGAAGGTTTTAAATGGGAACGTGTGATGAATTTTGAAGATGGTGGTGTTGTTA
CCGTTACACAGGATAGCAGCCTGCAGGATGGTGAATTTATCTATAAAGTTAAACTGCGTGGCACGAATTTTCCGAGT
GATGGTCCGGTTATGCAGAAAAAAACCATGGGTTGGGAAGCAAGCAGCGAACGTATGTATCCGGAAGATGGCGCACT
GAAAGGTGAAATTAAACAGCGTCTGAAGCTGAAAGATGGCGGTCATTATGATGCAGAAGTTAAAACCACCTACAAAG
CCAAAAAACCGGTTCAGCTGCCTGGTGCATATAACGTTAACATTAAACTGGATATCACCAGCCACAACGAGGATTAT
ACCATTGTTGAACAGTATGAACGTGCAGAAGGTCGCCATAGTACCGGTGGTATGGATGAACTGTATAAA
(LbCas12a (E795L)-A(EAAAK)₄ALEA(EAAAK)₄A(SEQ ID NO: 9)-
mCherry chimera DNA sequence)
SEQ ID NO: 414
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCCGAAGCAGCA
GCCAAAGAAGCTGCCGCAAAGGAGGCGGCAGCTAAAGAAGCGGCCGCTAAGGCATTGGAAGCGGAGGCGGCTGCCAA
GGAGGCTGCGGCGAAAGAAGCTGCGGCCAAAGAAGCGGCTGCAAAAGCGATGGTGAGCAAAGGTGAAGAGGATAATA
TGGCCATCATCAAAGAATTCATGCGCTTCAAAGTTCATATGGAAGGTAGCGTTAATGGCCACGAATTTGAAATTGAA
GGTGAAGGCGAAGGTCGTCCGTATGAAGGCACCCAGACCGCAAAACTGAAAGTTACCAAAGGTGGTCCGCTGCCGTT
TGCATGGGATATTCTGAGTCCGCAGTTTATGTATGGTAGCAAAGCCTATGTTAAACATCCGGCAGATATCCCGGATT
ATCTGAAACTGAGCTTTCCGGAAGGTTTTAAATGGGAACGTGTGATGAATTTTGAAGATGGTGGTGTTGTTACCGTT
ACACAGGATAGCAGCCTGCAGGATGGTGAATTTATCTATAAAGTTAAACTGCGTGGCACGAATTTTCCGAGTGATGG
TCCGGTTATGCAGAAAAAAACCATGGGTTGGGAAGCAAGCAGCGAACGTATGTATCCGGAAGATGGCGCACTGAAAG
GTGAAATTAAACAGCGTCTGAAGCTGAAAGATGGCGGTCATTATGATGCAGAAGTTAAAACCACCTACAAAGCCAAA
AAACCGGTTCAGCTGCCTGGTGCATATAACGTTAACATTAAACTGGATATCACCAGCCACAACGAGGATTATACCAT
TGTTGAACAGTATGAACGTGCAGAAGGTCGCCATAGTACCGGTGGTATGGATGAACTGTATAAA
(LbCas12a (E795L)-LEA(EAAAK)₄ALEA(EAAAK)₄ALE(SEQ ID NO: 10)-
mCherry chimera DNA sequence)
SEQ ID NO: 415
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATTTAGAGGCTGAG
GCTGCTGCCAAGGAGGCTGCTGCGAAGGAAGCTGCAGCTAAAGAGGCAGCGGCCAAAGCACTTGAGGCCGAAGCCGC
CGCGAAAGAAGCCGCTGCTAAGGAGGCAGCCGCCAAAGAAGCGGCAGCTAAAGCATTAGAAATGGTGAGCAAAGGTG
AAGAGGATAATATGGCCATCATCAAAGAATTCATGCGCTTCAAAGTTCATATGGAAGGTAGCGTTAATGGCCACGAA
TTTGAAATTGAAGGTGAAGGCGAAGGTCGTCCGTATGAAGGCACCCAGACCGCAAAACTGAAAGTTACCAAAGGTGG
TCCGCTGCCGTTTGCATGGGATATTCTGAGTCCGCAGTTTATGTATGGTAGCAAAGCCTATGTTAAACATCCGGCAG
ATATCCCGGATTATCTGAAACTGAGCTTTCCGGAAGGTTTTAAATGGGAACGTGTGATGAATTTTGAAGATGGTGGT
GTTGTTACCGTTACACAGGATAGCAGCCTGCAGGATGGTGAATTTATCTATAAAGTTAAACTGCGTGGCACGAATTT
TCCGAGTGATGGTCCGGTTATGCAGAAAAAAACCATGGGTTGGGAAGCAAGCAGCGAACGTATGTATCCGGAAGATG
GCGCACTGAAAGGTGAAATTAAACAGCGTCTGAAGCTGAAAGATGGCGGTCATTATGATGCAGAAGTTAAAACCACC
TACAAAGCCAAAAAACCGGTTCAGCTGCCTGGTGCATATAACGTTAACATTAAACTGGATATCACCAGCCACAACGA
GGATTATACCATTGTTGAACAGTATGAACGTGCAGAAGGTCGCCATAGTACCGGTGGTATGGATGAACTGTATAAA
(LbCas12a (E795L)-APA-mCherry chimera DNA sequence)
SEQ ID NO: 416
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCACCTGCGATG
GTGAGCAAAGGTGAAGAGGATAATATGGCCATCATCAAAGAATTCATGCGCTTCAAAGTTCATATGGAAGGTAGCGT
TAATGGCCACGAATTTGAAATTGAAGGTGAAGGCGAAGGTCGTCCGTATGAAGGCACCCAGACCGCAAAACTGAAAG
TTACCAAAGGTGGTCCGCTGCCGTTTGCATGGGATATTCTGAGTCCGCAGTTTATGTATGGTAGCAAAGCCTATGTT
AAACATCCGGCAGATATCCCGGATTATCTGAAACTGAGCTTTCCGGAAGGTTTTAAATGGGAACGTGTGATGAATTT
TGAAGATGGTGGTGTTGTTACCGTTACACAGGATAGCAGCCTGCAGGATGGTGAATTTATCTATAAAGTTAAACTGC
GTGGCACGAATTTTCCGAGTGATGGTCCGGTTATGCAGAAAAAAACCATGGGTTGGGAAGCAAGCAGCGAACGTATG
TATCCGGAAGATGGCGCACTGAAAGGTGAAATTAAACAGCGTCTGAAGCTGAAAGATGGCGGTCATTATGATGCAGA
AGTTAAAACCACCTACAAAGCCAAAAAACCGGTTCAGCTGCCTGGTGCATATAACGTTAACATTAAACTGGATATCA
CCAGCCACAACGAGGATTATACCATTGTTGAACAGTATGAACGTGCAGAAGGTCGCCATAGTACCGGTGGTATGGAT
GAACTGTATAAA
(LbCas12a (E795L)-APAPA(SEQ ID NO: 11)-mCherry chimera DNA sequence)
SEQ ID NO: 417
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTCCTGCACCT
GCGATGGTGAGCAAAGGTGAAGAGGATAATATGGCCATCATCAAAGAATTCATGCGCTTCAAAGTTCATATGGAAGG
TAGCGTTAATGGCCACGAATTTGAAATTGAAGGTGAAGGCGAAGGTCGTCCGTATGAAGGCACCCAGACCGCAAAAC
TGAAAGTTACCAAAGGTGGTCCGCTGCCGTTTGCATGGGATATTCTGAGTCCGCAGTTTATGTATGGTAGCAAAGCC
TATGTTAAACATCCGGCAGATATCCCGGATTATCTGAAACTGAGCTTTCCGGAAGGTTTTAAATGGGAACGTGTGAT
GAATTTTGAAGATGGTGGTGTTGTTACCGTTACACAGGATAGCAGCCTGCAGGATGGTGAATTTATCTATAAAGTTA
AACTGCGTGGCACGAATTTTCCGAGTGATGGTCCGGTTATGCAGAAAAAAACCATGGGTTGGGAAGCAAGCAGCGAA
CGTATGTATCCGGAAGATGGCGCACTGAAAGGTGAAATTAAACAGCGTCTGAAGCTGAAAGATGGCGGTCATTATGA
TGCAGAAGTTAAAACCACCTACAAAGCCAAAAAACCGGTTCAGCTGCCTGGTGCATATAACGTTAACATTAAACTGG
ATATCACCAGCCACAACGAGGATTATACCATTGTTGAACAGTATGAACGTGCAGAAGGTCGCCATAGTACCGGTGGT
ATGGATGAACTGTATAAA
(LbCas12a (E795L)-APAPAPA(SEQ ID NO: 12)-mCherry chimera DNA sequence)
SEQ ID NO: 418
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCTCCT
GCACCTGCGATGGTGAGCAAAGGTGAAGAGGATAATATGGCCATCATCAAAGAATTCATGCGCTTCAAAGTTCATAT
GGAAGGTAGCGTTAATGGCCACGAATTTGAAATTGAAGGTGAAGGCGAAGGTCGTCCGTATGAAGGCACCCAGACCG
CAAAACTGAAAGTTACCAAAGGTGGTCCGCTGCCGTTTGCATGGGATATTCTGAGTCCGCAGTTTATGTATGGTAGC
AAAGCCTATGTTAAACATCCGGCAGATATCCCGGATTATCTGAAACTGAGCTTTCCGGAAGGTTTTAAATGGGAACG
TGTGATGAATTTTGAAGATGGTGGTGTTGTTACCGTTACACAGGATAGCAGCCTGCAGGATGGTGAATTTATCTATA
AAGTTAAACTGCGTGGCACGAATTTTCCGAGTGATGGTCCGGTTATGCAGAAAAAAACCATGGGTTGGGAAGCAAGC
AGCGAACGTATGTATCCGGAAGATGGCGCACTGAAAGGTGAAATTAAACAGCGTCTGAAGCTGAAAGATGGCGGTCA
TTATGATGCAGAAGTTAAAACCACCTACAAAGCCAAAAAACCGGTTCAGCTGCCTGGTGCATATAACGTTAACATTA
AACTGGATATCACCAGCCACAACGAGGATTATACCATTGTTGAACAGTATGAACGTGCAGAAGGTCGCCATAGTACC
GGTGGTATGGATGAACTGTATAAA
(LbCas12a (E795L)-APAPAPAPA(SEQ ID NO: 13)-mCherry chimera DNA sequence)
SEQ ID NO: 419
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCTCCTGCGCCA
GCTCCTGCACCTGCGATGGTGAGCAAAGGTGAAGAGGATAATATGGCCATCATCAAAGAATTCATGCGCTTCAAAGT
TCATATGGAAGGTAGCGTTAATGGCCACGAATTTGAAATTGAAGGTGAAGGCGAAGGTCGTCCGTATGAAGGCACCC
AGACCGCAAAACTGAAAGTTACCAAAGGTGGTCCGCTGCCGTTTGCATGGGATATTCTGAGTCCGCAGTTTATGTAT
GGTAGCAAAGCCTATGTTAAACATCCGGCAGATATCCCGGATTATCTGAAACTGAGCTTTCCGGAAGGTTTTAAATG
GGAACGTGTGATGAATTTTGAAGATGGTGGTGTTGTTACCGTTACACAGGATAGCAGCCTGCAGGATGGTGAATTTA
TCTATAAAGTTAAACTGCGTGGCACGAATTTTCCGAGTGATGGTCCGGTTATGCAGAAAAAAACCATGGGTTGGGAA
GCAAGCAGCGAACGTATGTATCCGGAAGATGGCGCACTGAAAGGTGAAATTAAACAGCGTCTGAAGCTGAAAGATGG
CGGTCATTATGATGCAGAAGTTAAAACCACCTACAAAGCCAAAAAACCGGTTCAGCTGCCTGGTGCATATAACGTTA
ACATTAAACTGGATATCACCAGCCACAACGAGGATTATACCATTGTTGAACAGTATGAACGTGCAGAAGGTCGCCAT
AGTACCGGTGGTATGGATGAACTGTATAAA
(LbCas12a (E795L)-APAPAPAPAPA(SEQ ID NO: 14)-mCherry chimera DNA sequence)
SEQ ID NO: 420
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCCGCTCCT
GCGCCAGCTCCTGCACCTGCGATGGTGAGCAAAGGTGAAGAGGATAATATGGCCATCATCAAAGAATTCATGCGCTT
CAAAGTTCATATGGAAGGTAGCGTTAATGGCCACGAATTTGAAATTGAAGGTGAAGGCGAAGGTCGTCCGTATGAAG
GCACCCAGACCGCAAAACTGAAAGTTACCAAAGGTGGTCCGCTGCCGTTTGCATGGGATATTCTGAGTCCGCAGTTT
ATGTATGGTAGCAAAGCCTATGTTAAACATCCGGCAGATATCCCGGATTATCTGAAACTGAGCTTTCCGGAAGGTTT
TAAATGGGAACGTGTGATGAATTTTGAAGATGGTGGTGTTGTTACCGTTACACAGGATAGCAGCCTGCAGGATGGTG
AATTTATCTATAAAGTTAAACTGCGTGGCACGAATTTTCCGAGTGATGGTCCGGTTATGCAGAAAAAAACCATGGGT
TGGGAAGCAAGCAGCGAACGTATGTATCCGGAAGATGGCGCACTGAAAGGTGAAATTAAACAGCGTCTGAAGCTGAA
AGATGGCGGTCATTATGATGCAGAAGTTAAAACCACCTACAAAGCCAAAAAACCGGTTCAGCTGCCTGGTGCATATA
ACGTTAACATTAAACTGGATATCACCAGCCACAACGAGGATTATACCATTGTTGAACAGTATGAACGTGCAGAAGGT
CGCCATAGTACCGGTGGTATGGATGAACTGTATAAA
(LbCas12a (E795L)-APAPAPAPAPAPA(SEQ ID NO: 15)-mCherry chimera DNA sequence)
SEQ ID NO: 421
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCACCTGCGATGGTGAGCAAAGGTGAAGAGGATAATATGGCCATCATCAAAGAATTCAT
GCGCTTCAAAGTTCATATGGAAGGTAGCGTTAATGGCCACGAATTTGAAATTGAAGGTGAAGGCGAAGGTCGTCCGT
ATGAAGGCACCCAGACCGCAAAACTGAAAGTTACCAAAGGTGGTCCGCTGCCGTTTGCATGGGATATTCTGAGTCCG
CAGTTTATGTATGGTAGCAAAGCCTATGTTAAACATCCGGCAGATATCCCGGATTATCTGAAACTGAGCTTTCCGGA
AGGTTTTAAATGGGAACGTGTGATGAATTTTGAAGATGGTGGTGTTGTTACCGTTACACAGGATAGCAGCCTGCAGG
ATGGTGAATTTATCTATAAAGTTAAACTGCGTGGCACGAATTTTCCGAGTGATGGTCCGGTTATGCAGAAAAAAACC
ATGGGTTGGGAAGCAAGCAGCGAACGTATGTATCCGGAAGATGGCGCACTGAAAGGTGAAATTAAACAGCGTCTGAA
GCTGAAAGATGGCGGTCATTATGATGCAGAAGTTAAAACCACCTACAAAGCCAAAAAACCGGTTCAGCTGCCTGGTG
CATATAACGTTAACATTAAACTGGATATCACCAGCCACAACGAGGATTATACCATTGTTGAACAGTATGAACGTGCA
GAAGGTCGCCATAGTACCGGTGGTATGGATGAACTGTATAAA
(LbCas12a (E795L)-APAPAPAPAPAPAPA(SEQ ID NO: 16)-
mCherry chimera DNA sequence)
SEQ ID NO: 422
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCGATGGTGAGCAAAGGTGAAGAGGATAATATGGCCATCATCAAAGA
ATTCATGCGCTTCAAAGTTCATATGGAAGGTAGCGTTAATGGCCACGAATTTGAAATTGAAGGTGAAGGCGAAGGTC
GTCCGTATGAAGGCACCCAGACCGCAAAACTGAAAGTTACCAAAGGTGGTCCGCTGCCGTTTGCATGGGATATTCTG
AGTCCGCAGTTTATGTATGGTAGCAAAGCCTATGTTAAACATCCGGCAGATATCCCGGATTATCTGAAACTGAGCTT
TCCGGAAGGTTTTAAATGGGAACGTGTGATGAATTTTGAAGATGGTGGTGTTGTTACCGTTACACAGGATAGCAGCC
TGCAGGATGGTGAATTTATCTATAAAGTTAAACTGCGTGGCACGAATTTTCCGAGTGATGGTCCGGTTATGCAGAAA
AAAACCATGGGTTGGGAAGCAAGCAGCGAACGTATGTATCCGGAAGATGGCGCACTGAAAGGTGAAATTAAACAGCG
TCTGAAGCTGAAAGATGGCGGTCATTATGATGCAGAAGTTAAAACCACCTACAAAGCCAAAAAACCGGTTCAGCTGC
CTGGTGCATATAACGTTAACATTAAACTGGATATCACCAGCCACAACGAGGATTATACCATTGTTGAACAGTATGAA
CGTGCAGAAGGTCGCCATAGTACCGGTGGTATGGATGAACTGTATAAA
(LbCas12a (E795L)-APAPAPAPAPAPAPAPA(SEQ ID NO: 17)-
mCherry chimera DNA sequence)
SEQ ID NO: 423
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCGCCAGCTCCTGCACCTGCGATGGTGAGCAAAGGTGAAGAGGATAATATGGCCATCAT
CAAAGAATTCATGCGCTTCAAAGTTCATATGGAAGGTAGCGTTAATGGCCACGAATTTGAAATTGAAGGTGAAGGCG
AAGGTCGTCCGTATGAAGGCACCCAGACCGCAAAACTGAAAGTTACCAAAGGTGGTCCGCTGCCGTTTGCATGGGAT
ATTCTGAGTCCGCAGTTTATGTATGGTAGCAAAGCCTATGTTAAACATCCGGCAGATATCCCGGATTATCTGAAACT
GAGCTTTCCGGAAGGTTTTAAATGGGAACGTGTGATGAATTTTGAAGATGGTGGTGTTGTTACCGTTACACAGGATA
GCAGCCTGCAGGATGGTGAATTTATCTATAAAGTTAAACTGCGTGGCACGAATTTTCCGAGTGATGGTCCGGTTATG
CAGAAAAAAACCATGGGTTGGGAAGCAAGCAGCGAACGTATGTATCCGGAAGATGGCGCACTGAAAGGTGAAATTAA
ACAGCGTCTGAAGCTGAAAGATGGCGGTCATTATGATGCAGAAGTTAAAACCACCTACAAAGCCAAAAAACCGGTTC
AGCTGCCTGGTGCATATAACGTTAACATTAAACTGGATATCACCAGCCACAACGAGGATTATACCATTGTTGAACAG
TATGAACGTGCAGAAGGTCGCCATAGTACCGGTGGTATGGATGAACTGTATAAA
(LbCas12a (E795L)-APAPAPAPAPAPAPAPAPA-(SEQ ID NO: 18)
mCherry chimera DNA sequence)
SEQ ID NO: 424
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCGCCAGCTCCTGCACCTGCGATGGTGAGCAAAGGTGAAGAGGATAATATGGC
CATCATCAAAGAATTCATGCGCTTCAAAGTTCATATGGAAGGTAGCGTTAATGGCCACGAATTTGAAATTGAAGGTG
AAGGCGAAGGTCGTCCGTATGAAGGCACCCAGACCGCAAAACTGAAAGTTACCAAAGGTGGTCCGCTGCCGTTTGCA
TGGGATATTCTGAGTCCGCAGTTTATGTATGGTAGCAAAGCCTATGTTAAACATCCGGCAGATATCCCGGATTATCT
GAAACTGAGCTTTCCGGAAGGTTTTAAATGGGAACGTGTGATGAATTTTGAAGATGGTGGTGTTGTTACCGTTACAC
AGGATAGCAGCCTGCAGGATGGTGAATTTATCTATAAAGTTAAACTGCGTGGCACGAATTTTCCGAGTGATGGTCCG
GTTATGCAGAAAAAAACCATGGGTTGGGAAGCAAGCAGCGAACGTATGTATCCGGAAGATGGCGCACTGAAAGGTGA
AATTAAACAGCGTCTGAAGCTGAAAGATGGCGGTCATTATGATGCAGAAGTTAAAACCACCTACAAAGCCAAAAAAC
CGGTTCAGCTGCCTGGTGCATATAACGTTAACATTAAACTGGATATCACCAGCCACAACGAGGATTATACCATTGTT
GAACAGTATGAACGTGCAGAAGGTCGCCATAGTACCGGTGGTATGGATGAACTGTATAAA
(LbCas12a (E795L)-APAPAPAPAPAPAPAPAPAPA-(SEQ ID NO: 19)
mCherry chimera DNA sequence)
SEQ ID NO: 425
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCTCCTGCGCCAGCTCCTGCACCTGCGATGGTGAGCAAAGGTGAAGAGGATAA
TATGGCCATCATCAAAGAATTCATGCGCTTCAAAGTTCATATGGAAGGTAGCGTTAATGGCCACGAATTTGAAATTG
AAGGTGAAGGCGAAGGTCGTCCGTATGAAGGCACCCAGACCGCAAAACTGAAAGTTACCAAAGGTGGTCCGCTGCCG
TTTGCATGGGATATTCTGAGTCCGCAGTTTATGTATGGTAGCAAAGCCTATGTTAAACATCCGGCAGATATCCCGGA
TTATCTGAAACTGAGCTTTCCGGAAGGTTTTAAATGGGAACGTGTGATGAATTTTGAAGATGGTGGTGTTGTTACCG
TTACACAGGATAGCAGCCTGCAGGATGGTGAATTTATCTATAAAGTTAAACTGCGTGGCACGAATTTTCCGAGTGAT
GGTCCGGTTATGCAGAAAAAAACCATGGGTTGGGAAGCAAGCAGCGAACGTATGTATCCGGAAGATGGCGCACTGAA
AGGTGAAATTAAACAGCGTCTGAAGCTGAAAGATGGCGGTCATTATGATGCAGAAGTTAAAACCACCTACAAAGCCA
AAAAACCGGTTCAGCTGCCTGGTGCATATAACGTTAACATTAAACTGGATATCACCAGCCACAACGAGGATTATACC
ATTGTTGAACAGTATGAACGTGCAGAAGGTCGCCATAGTACCGGTGGTATGGATGAACTGTATAAA
(LbCas12a (E795L)-APAPAPAPAPAPAPAPAPAPAPA(SEQ ID NO: 20)-
mCherry chimera DNA sequence)
SEQ ID NO: 426
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCACCTGCGATGGTGAGCAAAGGTGAAGA
GGATAATATGGCCATCATCAAAGAATTCATGCGCTTCAAAGTTCATATGGAAGGTAGCGTTAATGGCCACGAATTTG
AAATTGAAGGTGAAGGCGAAGGTCGTCCGTATGAAGGCACCCAGACCGCAAAACTGAAAGTTACCAAAGGTGGTCCG
CTGCCGTTTGCATGGGATATTCTGAGTCCGCAGTTTATGTATGGTAGCAAAGCCTATGTTAAACATCCGGCAGATAT
CCCGGATTATCTGAAACTGAGCTTTCCGGAAGGTTTTAAATGGGAACGTGTGATGAATTTTGAAGATGGTGGTGTTG
TTACCGTTACACAGGATAGCAGCCTGCAGGATGGTGAATTTATCTATAAAGTTAAACTGCGTGGCACGAATTTTCCG
AGTGATGGTCCGGTTATGCAGAAAAAAACCATGGGTTGGGAAGCAAGCAGCGAACGTATGTATCCGGAAGATGGCGC
ACTGAAAGGTGAAATTAAACAGCGTCTGAAGCTGAAAGATGGCGGTCATTATGATGCAGAAGTTAAAACCACCTACA
AAGCCAAAAAACCGGTTCAGCTGCCTGGTGCATATAACGTTAACATTAAACTGGATATCACCAGCCACAACGAGGAT
TATACCATTGTTGAACAGTATGAACGTGCAGAAGGTCGCCATAGTACCGGTGGTATGGATGAACTGTATAAA
(LbCas12a (E795L)-APAPAPAPAPAPAPAPAPAPAPAPA(SEQ ID NO: 21)-
mCherry chimera DNA sequence)
SEQ ID NO: 427
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCTCCTGCACCTGCGATGGTGAGCAAAGG
TGAAGAGGATAATATGGCCATCATCAAAGAATTCATGCGCTTCAAAGTTCATATGGAAGGTAGCGTTAATGGCCACG
AATTTGAAATTGAAGGTGAAGGCGAAGGTCGTCCGTATGAAGGCACCCAGACCGCAAAACTGAAAGTTACCAAAGGT
GGTCCGCTGCCGTTTGCATGGGATATTCTGAGTCCGCAGTTTATGTATGGTAGCAAAGCCTATGTTAAACATCCGGC
AGATATCCCGGATTATCTGAAACTGAGCTTTCCGGAAGGTTTTAAATGGGAACGTGTGATGAATTTTGAAGATGGTG
GTGTTGTTACCGTTACACAGGATAGCAGCCTGCAGGATGGTGAATTTATCTATAAAGTTAAACTGCGTGGCACGAAT
TTTCCGAGTGATGGTCCGGTTATGCAGAAAAAAACCATGGGTTGGGAAGCAAGCAGCGAACGTATGTATCCGGAAGA
TGGCGCACTGAAAGGTGAAATTAAACAGCGTCTGAAGCTGAAAGATGGCGGTCATTATGATGCAGAAGTTAAAACCA
CCTACAAAGCCAAAAAACCGGTTCAGCTGCCTGGTGCATATAACGTTAACATTAAACTGGATATCACCAGCCACAAC
GAGGATTATACCATTGTTGAACAGTATGAACGTGCAGAAGGTCGCCATAGTACCGGTGGTATGGATGAACTGTATAA
A
(LbCas12a (E795L)-APAPAPAPAPAPAPAPAPAPAPAPAPA(SEQ ID NO: 22)-
mCherry chimera DNA sequence)
SEQ ID NO: 428
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCGCCAGCTCCTGCACCTGCGATGGTGAG
CAAAGGTGAAGAGGATAATATGGCCATCATCAAAGAATTCATGCGCTTCAAAGTTCATATGGAAGGTAGCGTTAATG
GCCACGAATTTGAAATTGAAGGTGAAGGCGAAGGTCGTCCGTATGAAGGCACCCAGACCGCAAAACTGAAAGTTACC
AAAGGTGGTCCGCTGCCGTTTGCATGGGATATTCTGAGTCCGCAGTTTATGTATGGTAGCAAAGCCTATGTTAAACA
TCCGGCAGATATCCCGGATTATCTGAAACTGAGCTTTCCGGAAGGTTTTAAATGGGAACGTGTGATGAATTTTGAAG
ATGGTGGTGTTGTTACCGTTACACAGGATAGCAGCCTGCAGGATGGTGAATTTATCTATAAAGTTAAACTGCGTGGC
ACGAATTTTCCGAGTGATGGTCCGGTTATGCAGAAAAAAACCATGGGTTGGGAAGCAAGCAGCGAACGTATGTATCC
GGAAGATGGCGCACTGAAAGGTGAAATTAAACAGCGTCTGAAGCTGAAAGATGGCGGTCATTATGATGCAGAAGTTA
AAACCACCTACAAAGCCAAAAAACCGGTTCAGCTGCCTGGTGCATATAACGTTAACATTAAACTGGATATCACCAGC
CACAACGAGGATTATACCATTGTTGAACAGTATGAACGTGCAGAAGGTCGCCATAGTACCGGTGGTATGGATGAACT
GTATAAA
(LbCas12a (E795L)-APAPAPAPAPAPAPAPAPAPAPAPAPAPA(SEQ ID NO: 23)-
mCherry chimera DNA sequence)
SEQ ID NO: 429
ATGAGCAAACTGGAAAAGTTCACCAACTGTTATAGCCTGAGCAAAACCCTGCGTTTTAAAGCAATTCCGGTTGGTAA
AACCCAAGAGAACATTGATAATAAACGCCTGCTGGTCGAAGATGAAAAACGCGCTGAAGATTATAAAGGCGTGAAAA
AACTGCTGGATCGCTATTATCTGAGCTTCATTAACGATGTGCTGCACAGCATTAAACTGAAGAACCTGAACAACTAT
ATCAGCCTGTTTCGTAAAAAAACCCGCACCGAAAAAGAAAACAAAGAGCTGGAAAACCTGGAAATCAATCTGCGTAA
AGAAATCGCCAAAGCGTTTAAAGGTAACGAGGGTTATAAAAGCCTGTTCAAGAAAGACATCATCGAAACCATTCTGC
CGGAATTTCTGGATGATAAAGATGAAATTGCCCTGGTGAATAGCTTTAATGGCTTTACCACCGCATTTACCGGCTTT
TTTGATAATCGCGAAAACATGTTCAGCGAAGAAGCAAAAAGCACCAGCATTGCATTTCGCTGCATTAATGAAAATCT
GACCCGCTACATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGTTCTGACCCAA
GAAGGTATCGACGTTTATAACGCAATTATTGGTGGTTTTGTTACCGAAAGCGGTGAGAAAATCAAAGGCCTGAATGA
ATATATCAACCTGTATAACCAGAAAACCAAACAGAAACTGCCGAAATTCAAACCGCTGTATAAACAGGTTCTGAGCG
ATCGTGAAAGCCTGAGCTTTTATGGTGAAGGTTATACCAGTGATGAAGAGGTTCTGGAAGTTTTTCGTAACACCCTG
AATAAAAACAGCGAGATCTTTAGCAGCATCAAAAAGCTTGAGAAACTGTTCAAAAACTTTGATGAGTATAGCAGCGC
AGGCATCTTTGTTAAAAATGGTCCGGCAATTAGCACCATCAGCAAAGATATTTTTGGCGAATGGAATGTGATCCGCG
ATAAATGGAATGCCGAATATGATGATATCCACCTGAAAAAAAAGGCCGTGGTGACCGAGAAATATGAAGATGATCGT
CGTAAAAGCTTCAAGAAAATTGGTAGCTTTAGCCTGGAACAGCTGCAAGAATATGCAGATGCAGATCTGAGCGTTGT
GGAAAAACTGAAAGAAATCATCATTCAGAAGGTGGACGAGATCTATAAAGTTTATGGTAGCAGCGAAAAACTGTTCG
ATGCAGATTTTGTTCTGGAAAAAAGCCTGAAAAAGAATGATGCCGTTGTGGCCATTATGAAAGATCTGCTGGATAGC
GTTAAGAGCTTCGAGAATTACATCAAAGCCTTTTTTGGTGAGGGCAAAGAAACCAATCGTGATGAAAGTTTCTATGG
CGATTTTGTGCTGGCCTATGATATTCTGCTGAAAGTGGACCATATTTATGATGCCATTCGCAATTATGTTACCCAGA
AACCGTATAGCAAAGACAAGTTCAAACTGTACTTTCAGAACCCGCAGTTTATGGGTGGTTGGGATAAAGATAAAGAA
ACCGATTATCGTGCCACCATCCTGCGTTATGGTAGTAAATACTATCTGGCCATCATGGACAAAAAATACGCAAAATG
CCTGCAGAAAATCGACAAAGATGATGTGAATGGCAACTATGAAAAAATCAACTACAAACTGCTGCCTGGTCCGAATA
AAATGCTGCCGAAAGTGTTCTTTAGCAAGAAATGGATGGCCTATTATAACCCGAGCGAGGATATTCAAAAGATCTAC
AAAAATGGCACCTTTAAAAAGGGCGACATGTTCAATCTGAACGATTGCCACAAACTGATCGATTTCTTCAAAGATTC
AATTTCGCGTTATCCGAAATGGTCCAATGCCTATGATTTTAACTTTAGCGAAACCGAAAAATACAAAGACATTGCCG
GTTTTTATCGCGAAGTGGAAGAACAGGGCTATAAAGTGAGCTTTGAAAGCGCAAGCAAAAAAGAGGTTGATAAGCTG
GTTGAAGAGGGCAAACTGTATATGTTCCAGATTTACAACAAAGATTTTAGCGACAAAAGCCATGGCACCCCGAATCT
GCATACCATGTACTTTAAACTGCTGTTCGACGAAAATAACCATGGTCAGATTCGTCTGAGCGGTGGTGCCGAACTGT
TTATGCGTCGTGCAAGTCTGAAAAAAGAAGAACTGGTTGTTCATCCGGCAAATAGCCCGATTGCAAACAAAAATCCG
GACAATCCGAAAAAAACCACGACACTGAGCTATGATGTGTATAAAGACAAACGTTTTAGCGAGGATCAGTATCTGCT
GCATATCCCGATTGCCATCAATAAATGCCCGAAAAACATCTTTAAGATCAACACCGAAGTTCGCGTGCTGCTGAAAC
ATGATGATAATCCGTATGTGATTGGCATTGATCGTGGTGAACGTAACCTGCTGTATATTGTTGTTGTTGATGGTAAA
GGCAACATCGTGGAACAGTATAGTCTGAACGAAATTATCAACAACTTTAACGGCATCCGCATCAAAACCGACTATCA
TAGCCTGCTGGACAAGAAAGAAAAAGAACGTTTTGAAGCACGTCAGAACTGGACCAGTATTGAAAACATCAAAGAAC
TGAAAGCCGGTTATATTAGCCAGGTGGTTCATAAAATCTGTGAGCTGGTAGAAAAATACGATGCAGTTATTGCACTG
GAAGATCTGAATAGCGGTTTCAAAAATAGCCGTGTGAAAGTCGAAAAACAGGTGTATCAGAAATTCGAGAAAATGCT
GATCGACAAACTGAACTACATGGTCGACAAAAAAAGCAATCCGTGTGCAACCGGTGGTGCACTGAAAGGTTATCAGA
TTACCAACAAATTTGAAAGCTTTAAAAGCATGAGCACCCAGAACGGCTTTATCTTCTATATTCCGGCATGGCTGACC
AGCAAAATTGATCCGAGCACCGGTTTTGTGAACCTGCTGAAAACAAAATATACCTCCATTGCCGACAGCAAGAAGTT
TATTAGCAGCTTTGATCGCATTATGTATGTTCCGGAAGAGGACCTGTTTGAATTCGCACTGGATTACAAAAATTTCA
GCCGTACCGATGCCGACTACATCAAAAAATGGAAACTGTACAGCTATGGTAACCGCATTCGCATTTTTCGCAACCCG
AAGAAAAACAATGTGTTCGATTGGGAAGAAGTTTGTCTGACCAGCGCATATAAAGAACTTTTCAACAAATACGGCAT
CAACTATCAGCAGGGTGATATTCGTGCACTGCTGTGTGAACAGAGCGATAAAGCGTTTTATAGCAGTTTTATGGCAC
TGATGAGCCTGATGCTGCAGATGCGTAATAGCATTACCGGTCGCACCGATGTGGATTTTCTGATTAGTCCGGTGAAA
AATTCCGATGGCATCTTTTATGATAGCCGCAATTACGAAGCACAAGAAAATGCAATTCTGCCGAAAAACGCAGATGC
AAATGGTGCATATAACATTGCACGTAAAGTTCTGTGGGCAATTGGCCAGTTTAAGAAAGCAGAAGATGAGAAGCTGG
ACAAAGTGAAAATTGCGATCAGCAATAAAGAGTGGCTGGAATACGCACAGACCAGCGTTAAACATGCGCCAGCGCCT
GCTCCTGCACCCGCTCCAGCTCCTGCCCCAGCTCCAGCGCCCGCTCCTGCGCCAGCTCCTGCACCTGCTCCAGCGAT
GGTGAGCAAAGGTGAAGAGGATAATATGGCCATCATCAAAGAATTCATGCGCTTCAAAGTTCATATGGAAGGTAGCG
TTAATGGCCACGAATTTGAAATTGAAGGTGAAGGCGAAGGTCGTCCGTATGAAGGCACCCAGACCGCAAAACTGAAA
GTTACCAAAGGTGGTCCGCTGCCGTTTGCATGGGATATTCTGAGTCCGCAGTTTATGTATGGTAGCAAAGCCTATGT
TAAACATCCGGCAGATATCCCGGATTATCTGAAACTGAGCTTTCCGGAAGGTTTTAAATGGGAACGTGTGATGAATT
TTGAAGATGGTGGTGTTGTTACCGTTACACAGGATAGCAGCCTGCAGGATGGTGAATTTATCTATAAAGTTAAACTG
CGTGGCACGAATTTTCCGAGTGATGGTCCGGTTATGCAGAAAAAAACCATGGGTTGGGAAGCAAGCAGCGAACGTAT
GTATCCGGAAGATGGCGCACTGAAAGGTGAAATTAAACAGCGTCTGAAGCTGAAAGATGGCGGTCATTATGATGCAG
AAGTTAAAACCACCTACAAAGCCAAAAAACCGGTTCAGCTGCCTGGTGCATATAACGTTAACATTAAACTGGATATC
ACCAGCCACAACGAGGATTATACCATTGTTGAACAGTATGAACGTGCAGAAGGTCGCCATAGTACCGGTGGTATGGA
TGAACTGTATAAA

All references, including publications, patent applications, and patents cited herein are hereby incorporated by reference to the same extent as if each reference were individually and specifically indicated to be incorporated by reference and were set forth in its entirety herein.

### REFERENCES

1. Jinek, M., et al., A programmable dual-RNA-guided DNA endonuclease in adaptive bacterial immunity. Science, 2012. 337(6096): p. 816-21.
2. Rees, H. A. and D. R. Liu, Base editing: precision chemistry on the genome and transcriptome of living cells. Nat Rev Genet, 2018. 19(12): p. 770-788.
3. Komor, A. C., et al., Programmable editing of a target base in genomic DNA without double-stranded DNA cleavage. Nature, 2016. 533(7603): p. 420-4.
4. Gaudelli, N. M., et al., Programmable base editing of A*T to G*C in genomic DNA without DNA cleavage. Nature, 2017. 551(7681): p. 464-471.
5. Nishida, K., et al., Targeted nucleotide editing using hybrid prokaryotic and vertebrate adaptive immune systems. Science, 2016. 353(6305).
6. Qi, L. S., et al., Repurposing CRISPR as an RNA-guided platform for sequence specific control of gene expression. Cell, 2013. 152(5): p. 1173-83.
7. Gilbert, L. A., et al., CRISPR-mediated modular RNA-guided regulation of transcription in eukaryotes. Cell, 2013. 154(2): p. 442-51.
8. Gilbert, L. A., et al., Genome-Scale CRISPR-Mediated Control of Gene Repression and Activation. Cell, 2014. 159(3): p. 647-61.
9. Anzalone, A. V., et al., Search-and-replace genome editing without double-strand breaks or donor DNA. Nature, 2019. 576(7785): p. 149-157.
10. Cong, L., et al., Multiplex genome engineering using CRISPR/Cas systems. Science, 2013. 339(6121): p. 819-23.
11. Vakulskas, C. A., et al., A high-fidelity Cas9 mutant delivered as a ribonucleoprotein complex enables efficient gene editing in human hematopoietic stem and progenitor cells. Nat Med, 2018. 24(8): p. 1216-1224.
12. Slaymaker, I. M., et al., Rationally engineered Cas9 nucleases with improved specificity. Science, 2016. 351(6268): p. 84-8.
13. Kleinstiver, B. P., et al., High-fidelity CRISPR-Cas9 nucleases with no detectable genome-wide off-target effects. Nature, 2016. 529(7587): p. 490-5.
14. Chen, J. S., et al., Enhanced proofreading governs CRISPR-Cas9 targeting accuracy. Nature, 2017. 550(7676): p. 407-410.
15. McCormick, A. L., M. S. Thomas, and A. W. Heath, Immunization with an interferongamma-gp120fusion protein induces enhanced immune responses to human immunodeficiency virus gp120. J Infect Dis, 2001. 184(11): p. 1423-30.
16. Bai, Y. and W. C. Shen, Improving the oral efficacy of recombinant granulocyte colony-stimulating factor and transferrin fusion protein by spacer optimization. Pharm Res, 2006. 23(9): p. 2116-21.
17. Wriggers, W., S. Chakravarty, and P. A. Jennings, Control of protein functional dynamics by peptide linkers. Biopolymers, 2005. 80(6): p. 736-46.
18. Arai, R., et al., Design of the linkers which effectively separate domains of a bifunctional fusion protein. Protein Eng, 2001. 14(8): p. 529-32.
19. Marqusee, S. and R. L. Baldwin, Helix stabilization by Glu- . . . Lys+ salt bridges in short peptides of de novo design. Proc Natl Acad Sci USA, 1987. 84(24): p. 8898-902.
20. Bhandari, D. G., et al., 1H-NM Rstudy of mobility and conformational constraints within the proline-rich N-terminal of the LC1 alkali light chain of skeletal myosin. Correlation with similar segments in other protein systems. Eur J Biochem, 1986. 160(2): p. 349-56.
21. Chen, X., J. L. Zaro, and W. C. Shen, Fusion protein linkers: property, design and functionality. Adv Drug Deliv Rev, 2013. 65(10): p. 1357-69.
22. Brendan P. Cormack, Raphael H. Valdivia, Stanley Falkow, FACS-optimized mutants of the green fluorescent protein (GFP), Gene, 1996. 173 (1): 33-38.
23. Shaner N C, Campbell R E, Steinbach P A, Giepmans B N, Palmer A E, Tsien R Y. Improved monomeric red, orange and yellow fluorescent proteins derived from Discosoma sp. red fluorescent protein. Nat Biotechnol. 2004 December; 22(12):1567-72.
24. Peng, R., Li, Z., Xu, Y., He, S., Peng, Q., Wu, L. A., Wu, Y., Qi, J., Wang, P., Shi, Y. and Gao, G. F. Structural insight into multistage inhibition of CRISPR-Cas12a by AcrVA4, Proc. Natl. Acad. Sci. U.S.A. 2019 116(38):18928-18936.

1-24. (canceled)

25. A chimeric protein comprising a guided endonuclease protein, a universal linker, and a second protein.

26. The chimeric protein of claim 25, wherein the guided endonuclease protein is a Cas protein.

27. The chimeric protein of claim 26, wherein the Cas protein is selected from the group consisting of Cas9 protein, AsCas12a protein, LbCas12a protein, a variant of Cas9 protein, a variant of AsCas12a protein, and a variant of LbCas12a protein.

28. The chimeric protein of claim 26, wherein the Cas protein is selected from the group comprising SEQ ID NO: 1, 2, and 3.

29. The chimeric protein of claim 26, wherein the Cas protein is SEQ ID NO: 1.

30. The chimeric protein of claim 26, wherein the Cas protein is SEQ ID NO: 2.

31. The chimeric protein of claim 26, wherein the Cas protein is SEQ ID NO: 3.

32. The chimeric protein of claim 25, wherein the second protein is a fluorescent protein.

33. The chimeric protein of claim 32, wherein the fluorescent protein is selected from the group comprising SEQ ID NO: 4 and 5.

34. The chimeric protein of claim 32, wherein the fluorescent protein is SEQ ID NO: 4.

35. The chimeric protein of claim 32, wherein the fluorescent protein is SEQ ID NO: 5.

36. The chimeric protein of claim 25, wherein the universal linker is selected from a flexible linker, a rigid linker, and a mixed linker.

37. The chimeric protein of claim 36, wherein the universal linker comprises a flexible linker selected from the group consisting of SEQ ID NO: 6 and 7.

38. The chimeric protein of claim 36, wherein the universal linker comprises a rigid linker selected from the group consisting of XP, APA, and SEQ ID NO: 8-36.

39. The chimeric protein of claim 36, wherein the universal linker comprises a mixed linker of SEQ ID NO: 37.

40. The chimeric protein of claim 25, wherein the chimeric protein is selected from the group consisting of SEQ ID NO: 38-309.

41. An isolated nucleic acid, wherein the isolated nucleic acid encodes the chimeric protein according to claim 40.

42. The isolated nucleic acid of claim 41, wherein the isolated nucleic acid is codon optimized for expression in an organism or host cell.

43. The isolated nucleic acid of claim 42, wherein the organism or host cell is selected from E. coli or H. sapiens.

44. The isolated nucleic acid of claim 41, wherein the isolated nucleic acid is selected from the group consisting of SEQ ID NO: 348-387 and 389-429.

45-47. (canceled)
